SEQUENCE LISTING (1 sequence)

SEQ ID NO: 1 - DNA, 23 bp, synthetic construct
  source 1..23: mol_type = other DNA
  modified_base 1: mod_base = OTHER; note = Attached to 5-carboxytetramethylrhodamine-OOO-Arg Arg Arg, wherein O represents 8-amino-2,6,10-trioxaoctanoic acid
  modified_base 23: mod_base = OTHER; note = Attached to Arg
acccctatcacgattagcattaa

(19) United States
(12) Patent Application Publication
(10) Pub. No.: US 2024/0350421 A1
(43) Pub. Date: Oct. 24, 2024

(54) Cationic Polymeric Nanoparticles and Their Uses in Treating Diseases

(71) Applicant: University of Connecticut, Farmington, CT (US)

(72) Inventors: Raman Bahal, Glastonbury, CT (US); Aniket Wahane, Williamantic, CT (US); Shipra Malik, Storrs, CT (US); Vishal Kasina, Mansfield Center, CT (US)

(73) Assignee: University of Connecticut, Farmington, CT (US)

(21) Appl. No.: 18/606,592

(22) Filed: Mar. 15, 2024

Related U.S. Application Data
(63) Continuation of application No. PCT/US2022/043843, filed on Sep. 16, 2022.
(60) Provisional application No. 63/245,000, filed on Sep. 16, 2021.

(51) Int. Cl. A61K 9/51; A61K 31/337; A61P 35/00; C12N 15/113

(52) U.S. Cl. CPC A61K 9/5153; A61K 9/5192; A61K 31/337; A61P 35/00; C12N 15/113; C12N 2310/141

(57) ABSTRACT

The present invention provides cationic polymeric nanoparticles, and nanoparticle formulations thereof. The invention also provides methods for preparing cationic polymeric nanoparticles, and methods of treating diseases, reducing tumor growth, and increasing uptake of a therapeutic agent by a tumor cell in a subject in need thereof.

### RELATED APPLICATIONS

This application is a 35 § U.S.C. 111(a) continuation application which claims the benefit of priority to International Application No. PCT/US2022/043843, filed on Sep. 16, 2022, which, in turn, claims the benefit of priority to U.S. Provisional Application No. 63/245,000, filed on Sep. 16, 2021. The entire contents of each of the foregoing applications are incorporated herein by reference.

### STATEMENT OF GOVERNMENT INTEREST

This invention was made with Government support under CA241194 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created Mar. 14, 2024, is named 98121_00378_SL.xml and is 2,924 bytes in size.

### BACKGROUND OF THE INVENTION

Nanoparticles-based delivery strategies have garnered significant attention for the delivery of therapeutically active agents ranging from small molecules to synthetic nucleic acids. At the cellular level, uniform distribution of nanoparticles in the cytoplasm increases its efficacy; however, endosomal entrapment still poses a significant barrier (Smith et al. Bioconjug. Chem. 2019, 30 (2), 263-272). Various mechanical methods, electroporation, nucleofection, commercially available lipofectamine-based reagents, chloroquine, or high salt conditions to decrease the endosomal entrapment, have been introduced to increase cytosolic delivery. However, the clinical translation of these methods remains a challenge.

Poly(lactic-co-glycolic acid) (PLGA)-based nanoparticles (NPs) have been used to deliver chemotherapeutic agents, siRNAs, plasmid DNA, and, regular and chemically modified peptide nucleic acids (PNAs). Also, US-FDA approval of PLGA polymer has made it an attractive candidate for various diagnostic and theranostic clinical applications. However, PLGA NPs show modest delivery due to their negative zeta potential and endosomal entrapment. Hence several ligand coated-PLGA nanoformulations have been tested to enhance its cellular delivery. Antibody-coated PLGA NPs has shown promising results to some extent; however, engineering antibody-coated NPs is challenging to deliver therapeutically active agents (M. Cardoso et al. Curr. Med. Chem. 2012). Also, manufacturing and scale-up represent an additional challenge for the clinical viability of antibody-coated PLGA formulations.

Accordingly, there is an unmet need for additional formulations to generate easily scalable nanoparticles with increased cytosolic delivery, and better efficacy without compromising their safety profile.

### SUMMARY OF THE INVENTION

The present invention is based, at least in part, on the generation of novel cationic polymeric nanoparticles, e.g., the PLGA-histidine-based nanoparticles, which exhibit enhanced cellular delivery with minimal toxicity. In particular, the nanoparticles of the present invention have a uniform size distribution carrying minimal cationic charge to mitigate nonspecific toxicity, and show promising physico-biochemical features and transfection efficiency in both in vitro and in vivo studies.

The inventors of the present invention also surprisingly discovered that the use of acetone:dichloromethane (DCM) as a solvent mixture during the formulation process significantly improves the morphology and size distribution of the cationic polymeric nanoparticles, e.g., the PLGA-histidine-based NPs. Specifically, the cationic polymeric nanoparticles of the present invention were formulated using the double emulsion solvent evaporation method. PLGA:poly-L-histidine formulation disclosed herein relies on ionic interactions, and involves simple mixing of the poly-L-histidine and PLGA polymer. Furthermore, using different weight ratios of PLGA and poly-L-histidine, their surface charge density could be reduced without affecting its superior transfection efficiency as compared to other cationic carriers, such as polyethyleneimine (PEI) and lipofectamine, which have a higher surface charge.

As demonstrated in the Examples of the application, the cationic polymeric nanoparticles disclosed herein, e.g., the PLGA-histidine-based nanoparticles, are capable of delivering therapeutic agents, e.g., small molecules, e.g., paclitaxel, peptide nucleic acids (PNA), and miRNA mimics, e.g., a miRNA-34a mimic. Specifically, in vitro and in vivo assessments demonstrated that the cationic polymeric nanoparticles, e.g., the PLGA: poly-L-histidine nanoparticles, showed optimal encapsulation of a small molecule-based drug paclitaxel, or a PNA based nucleic acid analog targeting microRNA-155, or a miRNA-34a mimic, possess moderate surface charge density that can target the tumor, and inhibit tumor growth after systemic administration better than conventional PLGA formulations. The safety of the cationic polymeric nanoparticles, e.g., the PLGA-histidine-based nanoparticles, was also confirmed in vivo via multiple endpoints. This present invention provides essential guidance on PLGA-histidine based formulations to generate easily scalable nanoparticles of uniform size, low polydispersity index (PDI), increased cytosolic delivery, and optimal in vivo efficacy without compromising their safety profile.

Accordingly, in one aspect, the present invention provides a cationic polymeric nanoparticle comprising a cationic histidine peptide and a poly(lactic-co-glycolic acid) (PLGA) polymer, wherein the nanoparticle comprises a therapeutic agent.

In some embodiments, the cationic peptide comprises a poly-L-histidine peptide.

In some embodiments, the PLGA polymer and the histidine peptide are present at a ratio of about 1:1 to about 50:1, about 1:1 to about 20:1, about 1:1 to about 30:1 about 1:1 to about 40:1, about 1:1 to about 60:1, about 1:1 to about 80:1, or about 1:1 to about 100:1.

In some embodiments, the PLGA polymer and the histidine peptide are present at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 19:1, about 20:1, about 25:1, about 30:1, about 35:1, about 40:1, about 45:1, about 49:1, about 50:1, about 60:1, about 65:1, about 70:1, about 75:1, about 80:1, about 85:1, about 90:1, about 95:1, or about 100:1.

In some embodiments, the PLGA polymer and the histidine peptide are present at a ratio of about 3:2, about 4:1, about 19:1, or about 49:1. In certain embodiments, the PLGA polymer and the histidine peptide are present at a ratio of about 4:1. In some embodiments, the PLGA polymer and the histidine peptide are present at a ratio of about 49:1.

In some embodiments, the histidine peptide forms a cationic domain on the surface of the nanoparticle. In some embodiments, the cationic domain of the histidine peptide is about 0.1 nm to about 5 nm, about 0.1 nm to about 4 nm, about 0.1 nm to about 3 nm, about 0.1 nm to about 2 nm, or about 0.1 to about 1.0 nm in diameter. In some embodiments, the cationic domain of the histidine peptide is about 0.2 nm to about 5 nm, about 0.2 nm to about 4 nm, about 0.2 nm to about 3 nm, about 0.2 nm to about 2 nm, or about 0.2 to about 1.0 nm in diameter. In some embodiments, the cationic domain of the histidine peptide is about 0.3 nm to about 5 nm, about 0.3 nm to about 4 nm, about 0.3 nm to about 3 nm, about 0.3 nm to about 2 nm, or about 0.3 to about 1.0 nm in diameter. In some embodiments, the cationic domain of the histidine peptide is about 0.4 nm to about 5 nm, about 0.4 nm to about 4 nm, about 0.4 nm to about 3 nm, about 0.4 nm to about 2 nm, or about 0.4 to about 1.0 nm in diameter. In some embodiments, the cationic domain of the histidine peptide is about 0.5 nm to about 5 nm, about 0.5 nm to about 4 nm, about 0.5 nm to about 3 nm, about 0.5 nm to about 2 nm, or about 0.5 to about 1.0 nm in diameter.

In some embodiments, the cationic domain of the histidine peptide is about 0.1 nm, about 0.2 nm, about 0.3 nm, about 0.4 nm, about 0.5 nm, about 0.6 nm, about 0.7 nm, about 0.8 nm, about 0.9 nm, about 1.0 nm, about 1.1 nm, about 1.2 nm, about 1.3 nm, about 1.4 nm, about 1.5 nm, about 1.6 nm, about 1.7 nm, about 1.8 nm, about 1.9 nm, about 2.0 nm, about 2.1 nm, about 2.2 nm, about 2.3 nm, about 2.4 nm, about 2.5 nm, about 2.6 nm, about 2.7 nm, about 2.8 nm, about 2.9 nm, about 3.0 nm, about 3.1 nm, about 3.2 nm, about 3.3 nm, about 3.4 nm, about 3.5 nm, about 3.6 nm, about 3.7 nm, about 3.8 nm, about 3.9 nm, about 4.0 nm, about 4.1 nm, about 4.2 nm, about 4.3 nm, about 4.4 nm, about 4.5 nm, about 4.6 nm, about 4.7 nm, about 4.8 nm, about 4.9 nm, or about 5.0 nm, in diameter.

In some embodiments, the cationic domain of the histidine peptide is about 0.5 nm to about 5 nm in diameter. In some embodiments, the cationic domain of the histidine peptide is about 1.1 nm.

In some embodiments, the nanoparticle is about 100 nm to about 200 nm, about 120 nm to about 250 nm, about 150 to about 300 nm, or about 170 to about 200 nm in diameter. In some embodiments, the nanoparticle is about 100 nm, about 110 nm, about 120 nm, about 130 nm, about 140 nm, about 150 nm, about 155 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, or about 300 nm, in diameter. In some embodiments, the nanoparticle is about 170 nm to about 200 nm in diameter.

As used herein, the term “polydispersity index (PDI)” is used to estimate the average uniformity of a particle solution. PDI is defined as the standard deviation of the particle diameter distribution divided by the mean particle diameter. Larger PDI values correspond to a larger size distribution in the particle sample. PDI can also indicate nanoparticle aggregation along with the consistency and efficiency of particle surface modifications throughout the particle sample. A sample is considered monodisperse when the PDI value is less than 0.1 (J M Hughes, et al., J. Appl. Polym. Sci. 2014, 132(1): 41229).

In some embodiments, the nanoparticle has a polydispersity index (PDI) of about 0.10 nm to about 0.15 nm, about 0.10 nm to about 0.18, about 0.10 nm to about 0.20 nm, about 0.10 nm to about 0.30 nm, about 0.10 nm to about 0.40 nm, about 0.10 nm to about 0.50 nm, about 0.15 nm to about 0.25 nm, about 0.25 nm to about 0.35 nm, or about 0.35 nm about 0.50 nm. In some embodiments, the nanoparticle has a polydispersity index of about 0.10 nm to about 0.18 nm.

In some embodiments, the nanoparticle has a polydispersity index (PDI) of about 0.10 nm, about 0.11 nm, about 0.12 nm, about 0.13 nm, about 0.14 nm, about 0.15 nm, about 0.16 nm, about 0.17 nm, about 0.18 nm, about 0.19 nm, about 0.20 nm, about 0.21 nm, about 0.22 nm, about 0.23 nm, about 0.24 nm, about 0.25 nm, about 0.26 nm, about 0.27 nm, about 0.28 nm, about 0.29 nm, about 0.30 nm, about 0.31 nm, about 0.32 nm, about 0.33 nm, about 0.34 nm, about 0.35 nm, about 0.36 nm, about 0.37 nm, about 0.38 nm, about 0.39 nm, about 0.40 nm, about 0.41 nm, about 0.42 nm, about 0.43 nm, about 0.44 nm, about 0.45 nm, about 0.46 nm, about 0.47 nm, about 0.48 nm, about 0.49 nm, or about 0.50 nm.

In some embodiments, the therapeutic agent is selected from a group consisting of a small molecule, a nucleic acid, a peptide nucleic acid (PNA), a mRNA, a miRNA, a siRNA, a DNA mimic, a miRNA mimic, a protein, a peptide, an antibody, a lipid, and/or a combination of any of the foregoing.

In some embodiments, the therapeutic agent comprises a chemotherapeutic agent, a growth inhibitory agent, an anti-angiogenesis agent, an anti-neoplastic composition, and/or a combination of any of the foregoing.

In some embodiments, the therapeutic agent is paclitaxel.

In some embodiments, the therapeutic agent is a peptide nucleic acid. In some embodiments, the therapeutic agent is a peptide nucleic acid targeting miR-155 (PNA-155).

In some embodiments, the therapeutic agent is a miRNA or a miRNA mimic. In some embodiments, the therapeutic agent is miR-34a or miR-34a mimic. In some embodiments, the therapeutic agent is miR-24 or miR-24 mimic. In some embodiments, the therapeutic agent is miR-16 or miR-16 mimic.

In some embodiments, the nanoparticle is prepared using an organic solvent, wherein the organic solvent comprises acetone and/or dichloromethane.

In some embodiments, the acetone and dichloromethane are present in the organic solvent at a ratio of about 1:1 to about 20:1, about 1:1 to about 30:1 about 1:1 to about 40:1, about 1:1 to about 50:1, about 1:1 to about 60:1, about 1:1 to about 80:1, about 1:1 to about 100:1, about 1:1 to about 10:1, about 2:1 to about 50:1 about 2:1 to about 40:1, about 2:1 to about 30:1, about 2:1 to about 20:1, about 2:1 to about 10:1, about 2:1 to about 15:1, or about 2:1 to about 5:1.

In some embodiments, the acetone and dichloromethane are present in the organic solvent at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 20:1, or about 50:1.

In some embodiments, the acetone and dichloromethane are present in the organic solvent at a ratio of about 2:1.

In some embodiments, the nanoparticle is taken up by cells via clathrin-mediated endocytosis.

In one aspect, the present invention provides a pharmaceutical composition comprising the nanoparticle of the present invention, and a pharmaceutically acceptable excipient.

In another aspect, the present invention provides a method of preparing a cationic polymeric nanoparticle comprising a therapeutic agent comprising combining a cationic histidine peptide and a poly(lactic-co-glycolic acid) (PLGA) polymer in an organic solvent to form an organic phase.

In some embodiments, the method further comprises (a) dissolving the therapeutic agent in a first aqueous phase containing water; (b) combining the organic phase with the first aqueous phase; (c) subjecting the mixture of step (b) to sonication for a sufficient period of time to produce a water-in-oil emulsion; (d) combining the water-in-oil emulsion with a second aqueous phase containing polyvinyl alcohol; (e) subjecting the mixture of step (d) to sonication for a sufficient period of time to produce a water-in-oil-in-water emulsion; (f) combining the water-in-oil-in-water emulsion with a third aqueous phase containing polyvinyl alcohol; (g) allowing the organic solvent to evaporate; and (h) isolating the cationic polymeric nanoparticle.

In some embodiments, the cationic peptide comprises a poly-L-histidine peptide.

In some embodiments, the PLGA polymer and the histidine peptide are present at a ratio of about 1:1 to about 50:1, about 1:1 to about 20:1, about 1:1 to about 30:1 about 1:1 to about 40:1, about 1:1 to about 60:1, about 1:1 to about 80:1, or about 100:1.

In some embodiments, the PLGA polymer and the histidine peptide are present at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 19:1, about 20:1, about 25:1, about 30:1, about 35:1, about 40:1, about 45:1, about 49:1, about 50:1, about 60:1, about 65:1, about 70:1, about 75:1, about 80:1, about 85:1, about 90:1, about 95:1, or about 100:1.

In some embodiments, the PLGA polymer and the histidine peptide are present at a ratio of about 3:2, about 4:1, about 19:1, or about 49:1. In some embodiments, the PLGA polymer and the histidine peptide are present at a ratio of about 49:1.

In some embodiments, the organic solvent comprises acetone and/or dichloromethane.

In some embodiments, the acetone and dichloromethane are present in the organic solvent at a ratio of about 1:1 to about 20:1, about 1:1 to about 30:1 about 1:1 to about 40:1, about 1:1 to about 50:1, about 1:1 to about 60:1, about 1:1 to about 80:1, about 1:1 to about 100:1, about 1:1 to about 10:1, about 2:1 to about 50:1, about 2:1 to about 40:1, about 2:1 to about 30:1, about 2:1 to about 20:1, about 2:1 to about 10:1, about 2:1 to about 15:1, or about 2:1 to about 5:1.

In some embodiments, the acetone and dichloromethane are present in the organic solvent at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 20:1, or about 50:1.

In some embodiments, the acetone and dichloromethane are present in the organic solvent at a ratio of about 2:1.

In some embodiments, the second aqueous phase comprises about 1% to about 100%, about 1% to about 75%, about 1% to about 50%, about 1% to about 25%, about 1% to about 20%, about 1% to about 15%, or about 1% to about 10% polyvinyl alcohol. In some embodiments, the second aqueous phase comprises about 1%, about 2%, about 3%, about 4%, about 5%, about 6%, about 7%, about 8%, about 9% or about 10% polyvinyl alcohol. In some embodiments, the second aqueous phase comprises about 5% polyvinyl alcohol.

In some embodiments, the third aqueous phase comprises about 0.1% to about 10%, about 0.1% to about 8%, about 0.1% to about 5%, about 0.1% to about 2.5%, about 0.1% to about 2%, about 0.1% to about 1.5%, or about 0.1% to about 1% polyvinyl alcohol. In some embodiments, the third aqueous phase comprises about 0.1%, about 0.2%, about 0.3%, about 0.4%, about 0.5%, about 0.6%, about 0.7%, about 0.8%, about 0.9% or about 1.0% polyvinyl alcohol. In some embodiments, the third aqueous phase comprises about 0.3% polyvinyl alcohol.

In some embodiments, the therapeutic agent is selected from a small molecule, a nucleic acid, a peptide nucleic acid (PNA), a mRNA, a miRNA, a siRNA, a DNA mimic, a miRNA mimic, a protein, a peptide, an antibody, a lipid, and combinations thereof.

In some embodiments, the therapeutic agent comprises a chemotherapeutic agent, a growth inhibitory agent, an anti-angiogenesis agent, an anti-neoplastic composition, or a combination thereof.

In some embodiments, the therapeutic agent is paclitaxel.

In some embodiments, the therapeutic agent is a peptide nucleic acid. In some embodiments, the therapeutic agent is a peptide nucleic acid targeting miR-155 (PNA-155).

In some embodiments, the therapeutic agent is a miRNA or a miRNA mimic. In some embodiments, the therapeutic agent is miR-34a or miR-34a mimic. In some embodiments, the therapeutic agent is miR-24 or miR-24 mimic. In some embodiments, the therapeutic agent is miR-16 or miR-16 mimic.

In one aspect, the present invention provides a method of treating a disease in a subject in need thereof, comprising administering to the subject a therapeutically effective amount of the nanoparticle of the present invention, or the pharmaceutical composition of the present invention, thereby treating the disease in the subject in need thereof.

In some embodiments, the disease is cancer. In some embodiments, the disease is an autoimmune disease.

In another aspect, the present invention provides a method of reducing a tumor growth in a subject in need thereof, comprising administering to the subject a therapeutically effective amount of the nanoparticle of the present invention, or the pharmaceutical composition of the present invention, thereby reducing the tumor growth in the subject in need thereof.

In one aspect, the present invention provides a method of increasing uptake of a therapeutic agent by a cell in a subject in need thereof, comprising administering to the subject a therapeutically effective amount of the nanoparticle of the present invention, or the pharmaceutical composition of the present invention, thereby increasing uptake of the therapeutic agent by the cell in the subject in need thereof. In some embodiments, the cell is a tumor cell.

In some embodiments, the nanoparticle or the pharmaceutical composition is administered intravenously.

In another aspect, the present invention provides a nanoparticle formulation comprising a cationic polymeric nanoparticle and an organic solvent, wherein the cationic polymeric nanoparticle comprises a cationic histidine peptide and a poly(lactic-co-glycolic acid) (PLGA) polymer, and wherein the organic solvent comprises acetone and/or dichloromethane.

In some embodiments, the cationic peptide comprises a poly-L-histidine peptide.

In some embodiments, the PLGA polymer and the histidine peptide are present at a ratio of about 1:1 to about 50:1, about 1:1 to about 20:1, about 1:1 to about 30:1 about 1:1 to about 40:1, about 1:1 to about 60:1, about 1:1 to about 80:1, or about 100:1.

In some embodiments, the PLGA polymer and the histidine peptide are present at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 19:1, about 20:1, about 25:1, about 30:1, about 35:1, about 40:1, about 45:1, about 49:1, about 50:1, about 60:1, about 65:1, about 70:1, about 75:1, about 80:1, about 85:1, about 90:1, about 95:1, or about 100:1.

In some embodiments, the PLGA polymer and the histidine peptide are present at a ratio of about 3:2, about 4:1, about 19:1, or about 49:1.

In some embodiments, the PLGA polymer and the histidine peptide are present at a ratio of about 49:1.

In some embodiments, the acetone and dichloromethane are present in the organic solvent at a ratio of about 1:1 to about 20:1, about 1:1 to about 30:1, about 1:1 to about 40:1, about 1:1 to about 50:1, about 1:1 to about 60:1, about 1:1 to about 80:1, about 1:1 to about 100:1, about 1:1 to about 10:1, about 2:1 to about 50:1, about 2:1 to about 40:1, about 2:1 to about 30:1, about 2:1 to about 20:1, about 2:1 to about 10:1, about 2:1 to about 15:1, or about 2:1 to about 5:1.

In some embodiments, the acetone and dichloromethane are present in the organic solvent at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 20:1, or about 50:1.

In some embodiments, the acetone and dichloromethane are present in the organic solvent at a ratio of about 2:1.

The present invention is illustrated by the following drawings and detailed description, which do not limit the scope of the invention described in the claims.

### BRIEF DESCRIPTION OF DRAWINGS

FIG. 1A depicts a schematic showing chemical structure of PLGA and histidine components following their representative arrangement in nanoparticles. FIG. 1B depicts the steps involved in nanoparticles formulation by double emulsion solvent evaporation technique.

FIGS. 2A-2D depict characterization and cellular uptake of PLGA NPs formulated with DCM as the organic phase. FIG. 2A depicts hydrodynamic diameters of nanoparticle formulations. Results depict mean of n=3 and error bars indicate standard error mean. FIG. 2B depicts poly-dispersity index (PDI) of different formulations. Results depict mean of n=3. Error bars signify standard error mean. FIG. 2C depicts Zeta potential of different nanoparticle formulations in water. Results is mean of n=3. Error bars signify standard error mean. t-test was used for statistical analysis, **p<0.01, ***p<0.001, ****p<0.0001. F1: PLGA NPs; F2: PLGA:poly-L-histidine 4:1 NPs; F3: PLGA L-histidine covalently conjugated NPs; F4: PLGA L-histidine unconjugated NPs. FIG. 2D depicts confocal microscopy images of HeLa cells after incubation with different NP formulations. After 24 h, cells were fixed with 4% paraformaldehyde and permeabilized by 0.1% triton-X followed by staining of nucleus with DAPI. Green indicates Coumarin (C6) loaded NPs, blue indicates nucleus. Scale bar represents 30 m.

FIGS. 3A-3F depict characterization and cellular uptake of PLGA NPs formulated with acetone:DCM (2:1) as the organic phase. FIG. 3A depicts Scanning Electron Microscopy (SEM) and Transmission Electron Microscopy images of blank PLGA NPs.

FIG. 3B depicts hydrodynamic diameters of nanoparticle formulations. Results show as mean of n=3. Error bars indicate standard error mean. FIG. 3C depicts poly-dispersity index (PDI) of different PLGA histidine NPs. Results depict mean of replicate (n=3). Error bars indicate standard error mean. FIG. 3D depicts Zeta potential of different PLGA histidine NPs. Results are represented as mean (n=3). Error bars indicate standard error mean. t-test was used for statistical analysis, *p<0.05, ***p<0.001, ****p<0.0001. F1: PLGA NPs; F2: PLGA:poly-L-histidine 4:1 NPs; F3: PLGA L-histidine covalently conjugated NPs; F4: PLGA L-histidine unconjugated NPs. FIG. 3E depicts Confocal microscopy in HeLa cells showing higher cellular uptake for F2 nanoparticles. After 24 h, cells were fixed with 4% paraformaldehyde and permeabilized by 0.1% triton-X followed by staining of nucleus with DAPI. Green indicates C6 loaded NPs, blue indicates nucleus. Scale bar represents 30 m. FIG. 3F depicts representative flow cytometry histogram showing uptake of C6 loaded PLGA NPs formulations in HeLa cells after 24 h nanoparticle treatment.

FIG. 4A depicts Zeta potential of different PLGA:poly-L-histidine NPs with indicated weight ratios of poly-L-histidine formulated using acetone:DCM (2:1) as the organic phase. Results depict mean (n=3). Error bars indicate standard error mean. t-test was used for statistical analysis, **p<0.01, ****p<0.0001. FIG. 4B depicts Histogram from flow cytometry showing cellular uptake in HeLa cells of F1 and F2 nanoparticles at 24 hours. F2 formulation contains 4.9:0.1 weight ratio of PLGA and poly-L-histidine. FIG. 4C depicts confocal microscopy images of HeLa cells showing time dependent cellular uptake of F1 and F2 nanoparticles. DAPI was used for staining the nucleus. Green indicates C6 loaded NPs, blue indicates nucleus. Scale bar represents 30 m. FIG. 4D depicts histogram from flow cytometry showing temperature dependent uptake in HeLa cells treated with F1 and F2 formulations at 4° C. and 37° C. after 2 hours of incubation. FIG. 4E depicts histogram from flow cytometry showing uptake in HeLa cells of F1 and F2 nanoparticles with endocytosis inhibitors after 2 hours incubation. F1: PLGA NPs; F2: PLGA:poly-L-histidine 4.9:0.1 NPs; CPZ: Chlorpromazine.

FIG. 5A depicts contrast matched SANS patterns for F1 (red), F2 (orange), F3 (green) and F4 (blue) formulations. The solid black curve on F2 scattering pattern represents the best-fit result by using a hard-sphere structure factor. FIG. 5B depicts schematic representation for F2 formulation where blue and green circle represents PLGA particle and poly-L-histidine patches, respectively.

FIGS. 6A-6H depict characterization and efficacy of PNA-155 NPs formulated with acetone:DCM (2:1) as the organic phase. FIG. 6A depicts hydrodynamic diameters of F1 and F2 nanoparticle formulations containing PNA-155. Results signifies mean (n=3). Error bars indicate standard error mean. FIG. 6B depicts poly-dispersity index (PDI) of F1 and F2 nanoparticle formulations. Results shown as mean of n=3. Error bars indicate standard error mean. FIG. 6C depicts Zeta potential of F1 and F2 nanoparticle formulations. Results depict mean (n≥3) and error bars indicate standard error mean. t-test was used for statistical analysis, ****p<0.0001. FIG. 6D depicts loading of PNA-155 in F1 and F2 nanoparticles. Results signifies as mean of n=3. Error bars indicate standard error mean. t-test was used for statistical analysis, **p<0.01. FIG. 6E depicts % Cumulative release of PNA-155 from F1 and F2 nanoparticles in 1×PBS over a period of 24 hours. Error bars indicate standard error mean. FIG. 6F depicts confocal microscopy images of HeLa cells showing cellular uptake of PNA-155 F1 and F2 nanoparticles. Red indicates PNA-155 loaded NPs, blue indicates nuclei. Scale bar represents 50 m. FIG. 6G depicts histogram from flow cytometry showing cellular uptake in U2932 cells of F1 and F2 nanoparticles. FIG. 6H depicts miR-155 knockdown in U2932 cells following PNA-155 nanoparticle treatment quantified by qRT-PCR. Results are represented as mean of n=3. Error bars represent standard error mean. t-test was used for statistical analysis, ****p<0.0001. F1: PLGA NPs; F2: PLGA:poly-L-histidine 4.9:0.1 NPs.

FIG. 7A depicts IVIS imaging of harvested tumors from xenograft mice treated with nile red containing F2 NPs after 4 and 8 hours of systemic administration. FIG. 7B depicts confocal imaging of tumor sections after 4 and 8 hours of nile red F2 NPs systemic administration. Blue=nucleus, Red=nile red. Scale bar is 200 μm. FIG. 7C depicts the tumor volume fold change curve after systemic treatment with F1 and F2 NPs. Results depict mean of n=5. Error bars indicate standard error mean. t-test was used for statistical analysis, **p<0.01, ***p<0.001, ****p<0.0001. FIG. 7D depicts the relative expression levels of miR-155 from tumors treated with F2 and the control group. Results are represented as mean of n=5. Error bars indicate standard error mean. FIG. 7E depicts the relative expression levels of FOXO3A and BACH1 (miR-155 downstream targets) from tumors treated with F2. Results depict mean of n=3. Error bars indicate standard error mean. FIG. 7F depicts cell proliferation marker Ki67 immunostaining of tumor section from control and two different F2 NPs treated tumors. The scale bar represents 100 μm (magnification×10). FIG. 7G depicts the clinical chemistry of F1 and F2 NPs treated xenograft mice including white blood count (WBC), red blood count (RBC), hemoglobin (HGB), and platelets (PLT). Results depict mean (n≥3). Error bars indicate standard error mean. FIG. 7H depicts blood biochemistry analysis of control, F1 and F2 NPs treated xenograft mice including alanine aminotransferase (ALT), aspartate aminotransferase (AST), lactate dehydrogenase (LDH), and blood urea nitrogen (BUN). Results depict mean (n≥3). Error bars represent standard error mean. F1 is PLGA NPs containing PNA-155 and F2 is PNA-155 loaded PLGA:poly-L-histidine 4.9:0.1 NPs.

FIG. 8A depicts the tumor growth fold change curves after systemic treatment with paclitaxel loaded F1 and F2 NPs in comparison to the control group. Results represent mean (n=5) and error bars indicate SEM, ***p<0.001, ****p<0.0001. FIG. 8B depicts cell proliferation marker Ki67 immunostaining of tumor section of control and two different F2 NPs treated tumors. The scale bar represents 100 μm (10× magnification). FIG. 8C depicts the clinical chemistry of F1 and F2 NPs treated xenograft mice including white blood count (WBC), red blood count (RBC), hemoglobin (HGB), and platelets (PLT). Results shown as mean (n≥3). Error bars indicate SEM. FIG. 8D depicts blood biochemistry analysis of control, F1 and F2 NPs treated xenograft mice including alanine aminotransferase (ALT), aspartate aminotransferase (AST), lactate dehydrogenase (LDH), and blood urea nitrogen (BUN). Results depict mean (n≥3) and error bars indicate standard error mean. F1 is PLGA NPs inclosing PNA-155 and F2 is PNA-155 loaded PLGA:poly-L-histidine 4.9:0.1 NPs.

FIG. 9 depicts PLGA:poly-L-histidine NPs at different indicated poly-L-histidine weight ratios. Arrow indicates the insoluble precipitate formation in the primary emulsion during the formulation of PLGA:poly-L-histidine NPs.

FIG. 10 depicts confocal microscopy in HeLa cells showing similar cellular uptake of F2 formulation having different poly-L-histidine weight ratios. After 24 h, cells were fixed with 4% paraformaldehyde and permeabilized by 0.1% triton-X followed by staining of nucleus with DAPI. Green indicates C6 loaded NPs, blue indicates nucleus. Scale bar represents 30 km.

FIGS. 11A-11B depict cell viability studies for lipofectamine, blank F1 and F2 formulation. FIG. 11A depicts cell viability in PBMC cells following treatment with lipofectamine, F1 and F2 blank NPs. Cell viability was measured by trypan blue assay. Results depict mean (n=3) and error bars indicate standard error mean. t-test was used for statistical analysis, **p<0.01. FIG. 11B depicts cell viability in HEK293 cells following treatment with lipofectamine, F1 and F2 blank NPs. Cell viability was measured by MTT assay. Results depict mean (n=3) and error bars indicate standard error mean. t-test was used for statistical analysis, ****p<0.0001. F1: PLGA NPs; F2: PLGA:poly-L-histidine 4.9:0.1 NPs.

FIG. 12 depicts endotoxin assay for blank, PNA-155, PTX and NR containing nanoparticles. Results depict mean (n=3) and error bars indicate standard error mean. F1: PLGA NPs; F2: PLGA:poly-L-histidine 4.9:0.1 NPs; F3: PLGA L-histidine covalently conjugated NPs; F4: PLGA L-histidine unconjugated NPs.

FIG. 13 depicts confocal microscopy in HeLa cells showing effect of endocytosis inhibitors on cellular uptake of F2 formulation. After 2 h incubation with endocytosis inhibitors and F2 NPs, cells were fixed with 4% paraformaldehyde and permeabilized by 0.1% triton-X followed by staining of nucleus with DAPI. Green indicates C6 loaded NPs, blue indicates nucleus. Scale bar represents 30 m. F2: PLGA:poly-L-histidine 4.9:0.1 NPs.

FIG. 14 depicts the SANS data of F1, F2, and F4 in D2O over the full q range between 0.002 to 0.1 Å-1. F1: PLGA NPs; F2: PLGA:poly-L-histidine 4.9:0.1 NPs; F4: PLGA L-histidine unconjugated NPs.

FIG. 15 depicts cell proliferation assay on HeLa cells following treatment with paclitaxel (PTX) and PTX containing F2 NPs. Cell viability was measured by trypan blue assay. Results depict mean (n=3) and error bars indicate standard error mean. PTX F2: PTX PLGA:poly-L-histidine 4.9:0.1 NPs.

FIGS. 16A-16B depict annexin V apoptosis assay for blank F2 NPs and lipofectamine in HeLa cells. FIG. 16A depicts flow cytometry dot plots of control, blank F2, and lipofectamine treated HeLa cells. Q4 represents healthy cell population, while Q3 and Q2 represent cells undergoing early and late apoptosis respectively. FIG. 16B depicts the graph representing percentage of live cells in untreated, blank F2 NPs and lipofectamine treated HeLa cells. Results depict mean (n=3) and error bars indicate standard error mean. t-test was used for statistical analysis, ***p<0.001. F2: PLGA:poly-L-histidine 4.9:0.1 NPs.

FIGS. 17A-17B depict Annexin V apoptosis assay for PTX and PTX F2 NPs in HeLa cells. FIG. 17A depicts flow cytometry dot plots of control, PTX, and PTX F2 NPs treated HeLa cells. PTX F2 NPs induces early and late apoptosis in HeLa cells for as compared to control. Q4 represents healthy cell population, while Q3 and Q2 represent cells undergoing early and late apoptosis respectively. FIG. 17B depicts the graph representing fold change in apoptotic cells in PTX and PTX F2 NPs treated HeLa cells. Results depict mean (n=3) and error bars indicate standard error mean. t-test was used for statistical analysis, ***p<0.001. PTX: paclitaxel; PTX F2: Paclitaxel PLGA:poly-L-histidine 4.9:0.1 NPs.

FIG. 18 depicts quantification of nile red intensities in harvested tumor, liver, lungs, and kidney of xenograft mice after 4 and 8 hours of systemic administration of F2 NPs containing nile red. Results depict mean (n=2) and error bars indicate standard error mean. F2: PLGA:poly-L-histidine 4.9:0.1.

FIGS. 19A-19B depict schematic of in vivo efficacy studies in U2932 xenograft mice. FIG. 19A depicts different treatment groups comprising of U2932 xenograft mice for PNA-155 and PXT NPs efficacy studies. FIG. 19B depicts in vivo efficacy plan for PNA-155 NPs treatment group. (C) In vivo efficacy plan for PTX NPs treatment group.

FIG. 20 depicts the weight of untreated (control), F1 and F2 NPs containing PNA-155 treated xenograft mice during the tumor growth study. Results depict mean (n≥4) and error bars indicate standard error mean. F1: PLGA NPs; F2: PLGA:poly-L-histidine 4.9:0.1.

FIG. 21 depicts relative organ weights of mice treated with PNA-155 F1 and F2 NPs at the time of harvesting. Results depict mean (n≥3) and error bars indicate standard error mean. F1: PLGA NPs; F2: PLGA:poly-L-histidine 4.9:0.1 NPs.

FIG. 22 depicts complete blood count analysis of mice treated with PNA-155 F1 and F2 NPs at the time of sacking. (HCT: Hematocrit, MCV: Mean corpuscular volume, MCH: Mean corpuscular hemoglobin, MCHC: Mean corpuscular hemoglobin concentration). Results depict mean (n≥3) and error bars indicate standard error mean. F1: PLGA NPs; F2: PLGA:poly-L-histidine 4.9:0.1 NPs.

FIG. 23 depicts blood chemistry analysis of mice treated with PNA-155 F1 and F2 NPs at the time of sacking. Results depict mean (n≥3) and error bars indicate standard error mean. F1: PLGA NPs; F2: PLGA:poly-L-histidine 4.9:0.1 NPs.

FIG. 24 depicts H&E staining of liver, kidney, and spleen of untreated, paclitaxel F2 and PNA-155 F2 NPs treated mice at the end of efficacy studies. The scale bar represents 200 μm. F2: PLGA:poly-L-histidine 4.9:0.1 NPs.

FIG. 25 depicts the weight of untreated (control), F1 and F2 NPs containing Paclitaxel treated xenograft mice during the tumor growth study. Results depict mean (n≥4) and error bars indicate standard error mean. F1: PLGA NPs; F2: PLGA:poly-L-histidine 4.9:0.1 NPs.

FIG. 26 depicts relative organ weights of mice treated with F1 and F2 NPs containing paclitaxel. Results depict mean (n≥4) and error bars indicate standard error mean. F1: PLGA NPs; F2: PLGA:poly-L-histidine 4.9:0.1 NPs.

FIG. 27 depicts complete blood count analysis of mice treated with paclitaxel F1 and F2 NPs. (HCT: Hematocrit, MCV: Mean corpuscular volume, MCH: Mean corpuscular hemoglobin, MCHC: Mean corpuscular hemoglobin concentration). Results depict mean (n≥3) and error bars indicate standard error mean. F1: PLGA NPs; F2: PLGA:poly-L-histidine 4.9:0.1 NPs.

FIG. 28 depicts blood biochemistry analysis of mice treated with paclitaxel F1 and F2 NPs at the time of sacking. Results depict mean (n≥3) and error bars indicate standard error mean. F1: PLGA NPs; F2: PLGA:poly-L-histidine 4.9:0.1 NPs.

FIG. 29 depicts Coumarin 6 loading in PLGA NPs formulated with acetone:DCM (2:1) as the organic phase. Results depict mean (n≥3) and error bars indicate standard error mean. F1: PLGA NPs; F2: PLGA:poly-L-histidine 4.9:0.1 NPs.

FIGS. 30A-30E depict formulation of PLGA:PH-miR-34a NPs and biophysical characterization. FIG. 30A depicts double emulsion solvent evaporation technique. PLGA and poly-L-Histidine were solubilized at a 4.9:0.1 w/w ratio in Acetone:DCM (2:1) organic solvent. FIG. 30B depicts SEM and TEM images of miR-34a loaded PLGA:PH nanoparticles as well as the respective size distribution. Scale bar represents 100 nm. FIG. 30C depicts nanoparticle hydrodynamic size in nm and particle size distribution comparing Blank, miR-34a loaded, and Scr-miR-34a loaded nanoparticles. Surface charge density in mV. FIG. 30D depicts loading (picomoles/mg) and release kinetics as % cumulative release of miR-34a loaded and Scr-miR-34a mimic loaded nanoparticles in PBS. FIG. 30E depicts in vitro release RNA integrity. miRNA mimic released from nanoparticles over 48 hrs in PBS. Absorbance measured at 260 nm. Results qualitatively assessed by polyacrylamide gel electrophoresis.

FIG. 31A depicts the SAXS patterns of PLGA-poly-L-His NPs without and with miR-34a mimic. FIG. 31B depicts that the miR-34a mimic is binding around the surface of PLGA-poly-L-His NP based on the SAXS outcomes.

FIGS. 32A-32G depict in vitro efficacy of miR-34a NPs. FIG. 32A depicts cellular uptake of FITC conjugated miR-34a mimic formulations after 24 hrs in A549 cells using confocal microscopy. miR-34a-FITC delivered via PLGA-poly-L-His NPs at a 2 mg/ml dose. White arrow points to green puncti, representing miR-34a-FITC NPs undergoing endosomal entrapment. Image was taken at 100×. Scale bar represents 50 μM. FIG. 32B depicts stacked histogram of FACS analysis to quantify cellular uptake of miR-34a-FITC NPs after 24 hrs in A549 cells. FIG. 32C depicts miR-34a expression of RNA isolated from A549 cells after treated with Scr-miR-34a mimic and miR-34a mimic NPs for 24 hrs at a 2 mg/ml dose. Data is shown as n=3 and error bars indicate SEM. 1% Agarose gel of PCR product shown above data set. FIG. 32D depicts p53 gene expression of RNA isolated from A549 cells after treated with Scr-miR-34a mimic and miR-34a mimic NPs for 24 hrs at a 2 mg/ml dose. Data is shown as n=3 and error bars indicate SEM. 1% Agarose gel of PCR product shown above data set. FIG. 32E depicts Western blot of protein extracted from A549 cells treated with Scramble mimic and miR-34a mimic NPs for 24 hrs at a 2 mg/ml dose. Data shows p53 protein levels and is normalized to Vinculin levels. Data shows n=3 and error bars indicate SEM. Protein blots indicate protein intensity based on pixels per band. FIG. 32F depicts baseline p53 protein levels of A549 cells in 1% 02 hypoxic conditions for 24 hrs. Data shows p53 protein levels and is normalized to Vinculin levels. Protein blots indicate protein intensity based on pixels per band. FIG. 32G depicts p53 protein levels of A549 cells treated with Scr-miR-34a mimic and miR-34a mimic NPs for 24 hrs and incubated in 1% 02 conditions for 24 hrs. Data shows n=3 and error bars indicate SEM. Protein blots indicate protein intensity based on pixels per band.

FIGS. 33A-33D depict cell viability analysis of miR-34a NP treated A549 cells. FIG. 33A depicts apoptosis of miR-34a NP treated A549 cells using an Annexin/7-amino acid-actinomycin (7-AAD) based assay. Cells were treated with Blank (nonencapsulated) and miR-34a NPs and Scr-34a NPs for 24 hrs at a 2 mg/ml dose. The cells undergoing apoptosis and necrosis were stained with Annexin, which was labelled with Phycoerythrin (PE) and 7-AAD, which stain apoptotic and necrotic cells respectively. The quadrants of the dot plots represent necrotic (Q1), late-stage apoptosis (Q2), early apoptosis (Q3), and live cells (Q4). The data was quantified using FlowJo to calculate the total percentage Apoptotic/cells. The data is shown as n=3 and error bars represent SEM. FIG. 33B depicts Annexin-FITC based apoptosis assay. A549 cells were either untreated, treated with Blank, miR-34a, or Scr-34a NPs at a 2 mg/ml NP dose for 24 hrs. Apoptotic cells were then stained with Annexin-V, which was conjugated with FITC. The cells were imaged using confocal microscopy. Images shown are cells treated with Blank and miR-34a NPs. Green puncti represent apoptotic cells. Scale bar represents 50 μm. FIG. 33C depicts A549 Colony forming efficiency of miR-34a NP treated cells. Cells were treated were either untreated, treated with Blank NPs, miR-34a NPs, or Scr-34a NPs for 24 hrs at a 2 mg/ml dose. Treated cells were re-seeded and colonies were stained with crystal violet after 13 days of growth. Number of colonies was represented by the crystal violet stain. Bar graph shows data where number of colonies is normalized to untreated cells. Data represents n=3 and error bars represent SEM. FIG. 33D depicts dose dependent trypan blue-based cell viability assay. A549 cells were treated with different doses of miR-34a NPs (0.25, 0.5, 0.75, 1, 2 mg/ml) and normalized to Scr-34a NP treated cells at the same doses. Dead cells were stained with trypan blue and counted using a Bio-Rad cell counter. Data represents normalized cell viability with respect to Blank NP. Data is shown as n=3 and error bars represent SEM.

FIGS. 34A-34G depict intratumoral in-vivo efficacy studies. FIG. 34A depicts intratumoral biodistribution of miR-34a NPs. Survival curve for all tumors. Survival point was 2000 mm3 and plotted against number of days. FIG. 34B depicts histology of A549 xenograft tumors. Tumors were stained with H&E stain. Hematoxylin stains nucleus and eosin stains cytoplasm. Ki-67 stains proliferative cells. FIG. 34C depicts miR-34a gene expression in tumor samples. Data is plotted normalized to PBS treated tumors. Data is shown as n>5 and error bars represent SEM. FIG. 34D depicts p53 gene expression in tumor samples. Data is plotted normalized to PBS treated tumors. Data is shown as n>5 and error bars represent SEM. FIG. 34E depicts SIRT1 gene expression in tumor samples. Data is plotted normalized to PBS treated tumors. Data is shown as n>5 and error bars represent SEM. FIG. 34F depicts p53 protein analysis by Western blot. Vinculin was used as the endogeneous control. Band intensity was quantified with ImageJ software. FIG. 34G depicts SIRT1 protein analysis by Western blot. GAPDH was used as the endogeneous control. Band intensity was quantified with ImageJ software.

FIGS. 35A-35B depict route of endocytosis of miR-34a NPs. FIG. 35A depicts endocytosis of miR-34a NPs using confocal microscopy. A549 cells were pre-treated with genistein, amiloride, and chlorpromazine (CPZ) for 30 min. a 2 mg/mL NP dose of miR-34a-FITC NPs was used. Images were taken at 40× magnification and the scale bar represents 50 μm. Green puncta represent miR-34a-FITC NPs. Blue represents nuclei. FIG. 35B depicts endocytosis of miR-34a NPs using flow cytometry. Histogram showing cellular uptake when treated with different endocytosis inhibitors. FlowJo used for quantification of data.

FIG. 36 depicts time-dependent cellular uptake of miR-34a NPs. Cells were treated with miR-34a-FITC NPs for 2 hr, 4 hr, 6 hr, and 24 hrs. The scale bar represents 50 μm. Green puncta represent miR-34a-FITC NPs. Blue represents nuclei.

### DETAILED DESCRIPTION

The present invention is based, at least in part, on the generation of novel cationic polymeric nanoparticles, e.g., the PLGA-histidine-based nanoparticles, which exhibit enhanced cellular delivery with minimal toxicity. In particular, the nanoparticles of the present invention have a uniform size distribution carrying minimal cationic charge to mitigate nonspecific toxicity, and show promising physico-biochemical features and transfection efficiency in both in vitro and in vivo studies.

The inventors of the present invention also surprisingly discovered that the use of acetone:dichloromethane (DCM) as a solvent mixture during the formulation process significantly improves the morphology and size distribution of the cationic polymeric nanoparticles, e.g., the PLGA-histidine-based NPs. Specifically, the cationic polymeric nanoparticles of the present invention were formulated using the double emulsion solvent evaporation method. PLGA:poly-L-histidine formulation disclosed herein relies on ionic interactions, and involves simple mixing of the poly-L-histidine and PLGA polymer. Furthermore, using different weight ratios of PLGA and poly-L-histidine, their surface charge density could be reduced without affecting its superior transfection efficiency as compared to other cationic carriers, such as polyethyleneimine (PEI) and lipofectamine, which have a higher surface charge.

As demonstrated in the Examples of the application, the cationic polymeric nanoparticles disclosed herein, e.g., the PLGA-histidine-based nanoparticles, are capable of delivering therapeutic agents, e.g., small molecules, e.g., paclitaxel, peptide nucleic acids (PNA), and miRNA mimics, e.g., a miRNA-34a mimic. Specifically, in vitro and in vivo assessments demonstrated that the cationic polymeric nanoparticles, e.g., the PLGA: poly-L-histidine nanoparticles, showed optimal encapsulation of small molecule-based drug paclitaxel, and PNA based nucleic acid analog targeting microRNA-155 or miRNA mimic, possess moderate surface charge density that can target the tumor, and inhibit tumor growth after systemic administration better than conventional PLGA formulations. The safety of the cationic polymeric nanoparticles, e.g., the PLGA-histidine-based nanoparticles, was also confirmed in vivo via multiple endpoints. This present invention provides essential guidance on PLGA-histidine based formulations to generate easily scalable nanoparticles of uniform size, low polydispersity index (PDI), increased cytosolic delivery, and optimal in vivo efficacy without compromising their safety profile.

Accordingly, the present invention provides cationic polymeric nanoparticles and nanoparticle formulation thereof. The present invention also provides methods for treating diseases, methods of reducing tumor growth, and methods of increasing uptake of a therapeutic agent by a cell in a subject in need thereof, by administering to the subject a therapeutically effective amount of the nanoparticles of the present invention. In further embodiments, the present invention also provides methods of preparing cationic polymeric nanoparticles of the invention.

### I. Definitions

In order that the present invention may be more readily understood, certain terms are first defined. In addition, it should be noted that whenever a value or range of values of a parameter is recited, it is intended that values and ranges intermediate to the recited values are also part of this invention.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” means one element or more than one element.

The term “comprising” or “comprises” is used herein in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

As used herein, the term “nanoparticle” refers to any particle having a diameter of less than 1000 nm, e.g., about 2 nm to about 200 nm. Nanoparticles disclosed herein may include one, or more biocompaticle and/or biodegradable polymers, e.g., a poly(lactic-co-glycolic acid) (PLGA) polymer.

As used herein, the term “cationic” refers to an ion or group of ions having positive charges. A “cationic nanoparticle” refers to a nanoparticle that has a net positive charge. A “cationic peptide” refers to a peptide having a net positive charge.

As used herein, the term “subject” refers to an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird. In one embodiment, the subject is a mammal. In another embodiment, the subject is a human, such as a human being treated or assessed for a disease, e.g., cancer. In some embodiments, the subject is a female human. In other embodiments, the subject is a male human. In some embodiments, the subject is a non-binary human. In one embodiment, the subject is an adult subject. In another embodiment, the subject is a pediatric subject.

As used herein, the terms “treating” or “treatment” refer to a beneficial or desired result, such as reducing at least one sign or symptom of a disease or disorder in a subject, for example, cancer. Treatment also includes a reduction of one or more sign or symptoms associated with a disease, e.g., cancer. “Treatment” can also mean prolonging survival as compared to expected survival in the absence of treatment. By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%. Accordingly, as used herein, the term “treatment” or “treating” includes any administration of a compound described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

The terms “disease” or “condition” refer to a state of being or health status of a patient or subject capable of being treated with the compositions or methods provided herein. The disease may be a cancer. The disease may be an autoimmune disease. The disease may be an infectious disease, such as a viral disease. In some further instances, “cancer” refers to human cancers and carcinomas, sarcomas, adenocarcinomas, lymphomas, leukemias, etc., including solid and lymphoid cancers, such as, kidney, breast, lung, bladder, colon, ovarian, prostate, pancreas, stomach, brain, head and neck, skin, uterine, testicular, glioma, esophagus, and liver cancer, hepatocarcinoma, lymphoma, B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

### II. Nanoparticles of the Invention

The present invention is based, at least in part, on the generation of novel cationic polymeric nanoparticles, e.g., the PLGA-histidine-based nanoparticles, which exhibit enhanced cellular delivery with minimal toxicity. In particular, the nanoparticles of the present invention have a uniform size distribution carrying minimal cationic charge to mitigate nonspecific toxicity, and show promising physico-biochemical features and transfection efficiency in both in vitro and in vivo studies. As demonstrated in the Examples of the application, the cationic polymeric nanoparticles disclosed herein, e.g., the PLGA-histidine-based nanoparticles, are capable of delivering therapeutic agents, e.g., small molecules, e.g., paclitaxel, peptide nucleic acids (PNA), and miRNA mimic, e.g., miRNA-34a. Specifically, in vitro and in vivo assessments demonstrated that the cationic polymeric nanoparticles, e.g., the PLGA:poly-L-histidine nanoparticles, showed optimal encapsulation of small molecule-based drug paclitaxel, PNA based nucleic acid analog targeting microRNA-155 or miRNA mimic, possess moderate surface charge density that can target the tumor, and inhibit tumor growth after systemic administration better than conventional PLGA formulations. The safety of the cationic polymeric nanoparticles, e.g., the PLGA-histidine-based nanoparticles, was also confirmed in vivo via multiple endpoints. This present invention provides essential guidance on PLGA-histidine based formulations to generate easily scalable nanoparticles of uniform size, low polydispersity index (PDI), increased cytosolic delivery, and optimal in vivo efficacy without compromising their safety profile.

Accordingly, the present invention provides cationic polymeric nanoparticles, wherein the nanoparticles comprise a therapeutic agent.

In general, a “nanoparticle” refers to any particle having a diameter of less than 1000 nm, e.g., about 10 nm to about 300 nm. In some embodiments, the nanoparticles may have a diameter ranging from about 100 nm to about 200 nm, about 120 nm to about 250 nm, about 150 to about 300 nm, or about 170 to about 200 nm. In some embodiments, the nanoparticles have a diameter of about 100 nm, about 110 nm, about 120 nm, about 130 nm, about 140 nm, about 150 nm, about 155 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, or about 300 nm. In some embodiments, the nanoparticles have a diameter ranging from about 170 nm to about 200 nm.

As used herein, the term “polydispersity index (PDI)” is used to estimate the average uniformity of a particle solution. PDI is defined as the standard deviation of the particle diameter distribution divided by the mean particle diameter. Larger PDI values correspond to a larger size distribution in the particle sample. PDI can also indicate nanoparticle aggregation along with the consistency and efficiency of particle surface modifications throughout the particle sample. A sample is considered monodisperse when the PDI value is less than 0.1 (J M Hughes, et al., J. Appl. Polym. Sci. 2014, 132(1): 41229).

In some embodiments, the nanoparticle has a polydispersity index (PDI) of about 0.10 nm to about 0.15 nm, about 0.10 nm to about 0.18, about 0.10 nm to about 0.20 nm, about 0.10 nm to about 0.30 nm, about 0.10 nm to about 0.40 nm, about 0.10 nm to about 0.50 nm, about 0.15 nm to about 0.25 nm, about 0.25 nm to about 0.35 nm, or about 0.35 nm about 0.50 nm. In some embodiments, the nanoparticle has a polydispersity index of about 0.10 nm to about 0.18 nm.

In some embodiments, the nanoparticle has a polydispersity index (PDI) of about 0.10 nm, about 0.11 nm, about 0.12 nm, about 0.13 nm, about 0.14 nm, about 0.15 nm, about 0.16 nm, about 0.17 nm, about 0.18 nm, about 0.19 nm, about 0.20 nm, about 0.21 nm, about 0.22 nm, about 0.23 nm, about 0.24 nm, about 0.25 nm, about 0.26 nm, about 0.27 nm, about 0.28 nm, about 0.29 nm, about 0.30 nm, about 0.31 nm, about 0.32 nm, about 0.33 nm, about 0.34 nm, about 0.35 nm, about 0.36 nm, about 0.37 nm, about 0.38 nm, about 0.39 nm, about 0.40 nm, about 0.41 nm, about 0.42 nm, about 0.43 nm, about 0.44 nm, about 0.45 nm, about 0.46 nm, about 0.47 nm, about 0.48 nm, about 0.49 nm, or about 0.50 nm.

In some embodiments, the nanoparticles comprise a matrix of polymer. Any suitable polymers known in the art can be used in the disclosed nanoparticles. The term “polymer,” as used herein, refers to a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. In some cases, additional moieties may also be present in the polymer, for example, biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In some embodiments, the polymers comprise natural or unnatural (synthetic) polymers. In some embodiments, the polymers comprise homopolymers or copolymers comprising two or more monomers. Copolymers can be random, block, or comprise a combination of random and block sequences.

In some embodiments, the polymers for use in the nanoparticles of the invention can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, polymers for use in the nanoparticles of the invention are biocompatible polymers, i.e., the polymers that do not typically induce an adverse response when inserted or injected into a living subject, for example, without causing significant inflammation and/or acute rejection of the polymer by the immune system, for instance, via a T-cell response. Accordingly, the nanoparticles contemplated herein are non-immunogenic, i.e., eliciting either no, or only minimal levels of immune response when introduced in a subject.

Biocompatibility typically refers to the acute rejection of material by at least a portion of the immune system, i.e., a non-biocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells. Non-limiting examples of biocompatible polymers that may be useful in various embodiments include poly(lactic-co-glycolic acid) (PLGA), polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), polycaprolactone, or copolymers or derivatives including these and/or other polymers.

In certain embodiments, polymers for use in the nanoparticles of the invention are biodegradable, i.e., the polymers are able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

In some embodiments, polymers for use in the nanoparticles of the present invention may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers for use in the nanoparticles of the present invention can be cationic polymers. In general, cationic polymers are able to condense and/or protect negatively charged strands of nucleic acids (e.g., DNA, RNA, or derivatives thereof). Amine-containing polymers such as poly(lysine), polyethylene imine (PEI), and poly(amidoamine) dendrimers are contemplated for use, in some embodiments, in a disclosed particle.

In some embodiments, polymers for use in the nanoparticles of the invention can be degradable polyesters bearing cationic side chains. Examples of these polyesters include poly(L-lactide-co-L-lysine), poly(serine ester), poly(4-hydroxy-L-proline ester).

In some embodiments, polymers for use in the nanoparticles of the invention are poly(lactic-co-glycolic acid) (PLGA) polymers. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA can be characterized by a lactic acid:glycolic acid molar ratio of approximately 85:15, approximately 75:25, approximately 60:40, approximately 50:50, approximately 40:60, approximately 25:75, or approximately 15:85. In some embodiments, the molar ratio of lactic acid to glycolic acid monomers in the polymer of the particle may be selected to optimize for various parameters, such as water uptake, therapeutic agent release and/or polymer degradation kinetics.

In some embodiments, the nanoparticles of this invention comprise a cationic peptide, e.g., a cationic histidine peptide. In some embodiments, the cationic peptide comprises a poly-L-histidine peptide.

In some embodiments, the nanoparticles comprise a cationic histidine peptide and a poly(lactic-co-glycolic acid) (PLGA) polymer. The ratio of the cationic histidine peptide and the PLGA polymers can be varied. For example, the PLGA polymer and the histidine peptide are present at a ratio of about 1:1 to about 50:1, about 1:1 to about 20:1, about 1:1 to about 30:1 about 1:1 to about 40:1, about 1:1 to about 60:1, about 1:1 to about 80:1, or about 1:1 to about 100:1.

In some embodiments, the PLGA polymer and the histidine peptide are present at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 19:1, about 20:1, about 25:1, about 30:1, about 35:1, about 40:1, about 45:1, about 49:1, about 50:1, about 60:1, about 65:1, about 70:1, about 75:1, about 80:1, about 85:1, about 90:1, about 95:1, or about 100:1.

In some embodiments, the PLGA polymer and the histidine peptide are present at a ratio of about 3:2, about 4:1, about 19:1, or about 49:1. In certain embodiments, the PLGA polymer and the histidine peptide are present at a ratio of about 4:1. In some embodiments, the PLGA polymer and the histidine peptide are present at a ratio of about 49:1.

In some embodiments, the histidine peptide forms a cationic domain on the surface of the nanoparticle. In some embodiments, the cationic domain of the histidine peptide is about 0.1 nm to about 5 nm, about 0.1 nm to about 4 nm, about 0.1 nm to about 3 nm, about 0.1 nm to about 2 nm, or about 0.1 to about 1.0 nm in diameter. In some embodiments, the cationic domain of the histidine peptide is about 0.2 nm to about 5 nm, about 0.2 nm to about 4 nm, about 0.2 nm to about 3 nm, about 0.2 nm to about 2 nm, or about 0.2 to about 1.0 nm in diameter. In some embodiments, the cationic domain of the histidine peptide is about 0.3 nm to about 5 nm, about 0.3 nm to about 4 nm, about 0.3 nm to about 3 nm, about 0.3 nm to about 2 nm, or about 0.3 to about 1.0 nm in diameter. In some embodiments, the cationic domain of the histidine peptide is about 0.4 nm to about 5 nm, about 0.4 nm to about 4 nm, about 0.4 nm to about 3 nm, about 0.4 nm to about 2 nm, or about 0.4 to about 1.0 nm in diameter. In some embodiments, the cationic domain of the histidine peptide is about 0.5 nm to about 5 nm, about 0.5 nm to about 4 nm, about 0.5 nm to about 3 nm, about 0.5 nm to about 2 nm, or about 0.5 to about 1.0 nm in diameter.

In some embodiments, the cationic domain of the histidine peptide is about 0.1 nm, about 0.2 nm, about 0.3 nm, about 0.4 nm, about 0.5 nm, about 0.6 nm, about 0.7 nm, about 0.8 nm, about 0.9 nm, about 1.0 nm, about 1.1 nm, about 1.2 nm, about 1.3 nm, about 1.4 nm, about 1.5 nm, about 1.6 nm, about 1.7 nm, about 1.8 nm, about 1.9 nm, about 2.0 nm, about 2.1 nm, about 2.2 nm, about 2.3 nm, about 2.4 nm, about 2.5 nm, about 2.6 nm, about 2.7 nm, about 2.8 nm, about 2.9 nm, about 3.0 nm, about 3.1 nm, about 3.2 nm, about 3.3 nm, about 3.4 nm, about 3.5 nm, about 3.6 nm, about 3.7 nm, about 3.8 nm, about 3.9 nm, about 4.0 nm, about 4.1 nm, about 4.2 nm, about 4.3 nm, about 4.4 nm, about 4.5 nm, about 4.6 nm, about 4.7 nm, about 4.8 nm, about 4.9 nm, or about 5.0 nm, in diameter.

In some embodiments, the cationic domain of the histidine peptide is about 0.5 nm to about 5 nm in diameter. In some embodiments, the cationic domain of the histidine peptide is about 1.1 nm.

The cationic polymeric nanoparticles of the invention further comprise an agent, and are capable of mediating cellular uptake or delivery of the agent with minimal toxicity. In some embodiments, the nanoparticles are taken up by cells via clathrin-mediated endocytosis. The agent may be released in a controlled release manner from the nanoparticles and allowed to interact locally with a particular site, e.g., a tumor. The agent may also travel to a distant site once it is released from the nanoparticles, e.g., a different site from where it is released.

The term “controlled release,” as used herein, is generally meant to encompass release of a substance (e.g., a drug) at a selected site at a controllable in rate, interval, and/or amount. Controlled release encompasses, but is not necessarily limited to, substantially continuous delivery, patterned delivery (e.g., intermittent delivery over a period of time that is interrupted by regular or irregular time intervals), and delivery of a bolus of a selected substance (e.g., as a predetermined, discrete amount if a substance over a relatively short period of time (e.g., a few seconds or minutes)).

Any agents known in the art may be delivered by the nanoparticles of the present invention, and may include, but are not limited to, for example, therapeutic agents (e.g. anti-cancer agents), diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.). Exemplary agents to be delivered in accordance with the present invention include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, the agent to be delivered is an agent useful in the treatment of a disease, e.g., cancer. In some embodiments, the therapeutic agent comprises a chemotherapeutic agent, an inhibitor of an immune-inhibitory protein, an immune checkpoint inhibitor, a growth inhibitory agent, a cytokine modulator, an immunotherapeutic agent, an anti-angiogenesis agent, an anti-neoplastic composition, and/or a combination of any of the foregoing.

Chemotherapeutic agents include, for example, alkylating agents (e.g., cyclophosphamide, iphosphamide and the like), metabolism antagonists (e.g., methotrexate, 5-fluorouracil and the like), anticancer antibiotics (e.g., mitomycin, adriamycin and the like), vegetable-derived anticancer agents (e.g., vincristine, vindesine, taxol and the like), cisplatin, carboplatin, etoposide, a diterpene derivative or a taxane such as paclitaxel (or its derivatives such as DHA-paclitaxel or PG-paxlitaxel) or cabazitaxel, and the like. In some embodiments, the therapeutic agent is paclitaxel.

Exemplary immune-inhibitory proteins include, but are not limited to cytotoxic T-lymphocyte-associated antigen 4 (CTLA4), programmed cell death protein 1 (PD1), programmed cell death protein 1 ligand (PDL1), lymphocyte activation gene 3 (LAG3), T cell membrane protein 3 (TIM3), T cell membrane protein 4 (TIM4), V-Set Immunoregulatory Receptor (VISTA), B7-H2, B7-H3, B7-H4, B7-H6, inducible T cell costimulatory (ICOS), herpes virus entry mediator (HVEM), CD160, gp49B, PIR-B, KIR family receptors, TIM-1, B- and T-lymphocyte-associated protein (BTLA), SlRPalpha (CD47), CD48, 2B4 (CD244), B7.1, B7.2, leukocyte immunoglobulin like receptor B1 (ILT-2), leukocyte immunoglobulin like receptor B2 (ILT-4), T cell immunoreceptor with Ig and ITIM Domains (TIGIT), HERV-H LTR-associating 2 (HHLA2), butyrophilins, CD39, CD73, and adenosine A2a receptor (A2AR). PD-1 is a checkpoint protein on T cells, which keeps T cells from attacking cells in the body that express PD-L1. Some cancer cells overexpress PD-L1, which enables them to evade detection by T cells, and inhibit T cell responses. Inhibitors of PD-L1 and PD-1 can boost the immune response against cancer cells, and can synergistically promote tumor cell killing when used in conjunction with agents that inhibit the expression and/or activity of STUB1. Exemplary anti-PD-L1 inhibitory antibodies include, but are not limited to, atezolizumab (Genentech), avelumab (Pfizer), and durvalumab (AstraZeneca). Exemplary anti-PD-1 inhibitory antibodies include, but are not limited to, pembrolizumab (Merck) and nivolumab (Bristol-Myers Squibb).

Examplary cytokine modulators include, but are not limited to negative regulators of cytokines, e.g., protein tyrosine phosphatase non-receptor type 2 (PTPN2).

Immunotherapeutic agents include, for example, microorganisms or bacterial components (e.g., muramyl dipeptide derivative, picibanil and the like), polysaccharides having immune potentiating activity (e.g., lentinan, sizofilan, krestin and the like), cytokines obtained by a gene engineering technology (e.g., interferon, interleukin (IL) and the like), colony stimulating factors (e.g., granulocyte colony stimulating factor, erythropoetin and the like) and the like, among these substances, those preferred are IL-1, IL-2, IL-12 and the like.

In some embodiments, the therapeutic agent is selected from a group consisting of a small molecule, a nucleic acid, a peptide nucleic acid (PNA), a mRNA, a miRNA, a siRNA, a DNA mimic, a miRNA mimic, a protein, a peptide, an antibody, a lipid, and/or a combination of any of the foregoing.

In some embodiments, the therapeutic agent comprises peptide nucleic acids. Peptide nucleic acids (PNAs) are nucleic acid mimics, e.g., DNA mimics, in which the deoxyribose phosphate backbone is replaced by a pseudopeptide backbone and only the four natural nucleobases are retained (see Hyrup et al., Bioorganic & Medicinal Chem. 4(1): 5-23, 1996). The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. U.S.A. 93:14670-675, 1996.

PNAs can be modified, e.g., to enhance their stability or cellular uptake, by attaching lipophilic or other helper groups to PNA, by the formation of PNA-DNA chimeras, or by the use of liposomes or other techniques of delivery known in the art. For example, PNA-DNA chimeras can be generated which may combine the advantageous properties of PNA and DNA. Such chimeras allow DNA recognition enzymes, e.g., RNAse H, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., Nucleic Acids Res. 24:3357-63, 1996. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., Nucleic Acids Res., 17:5973-88, 1989). PNA monomers are then coupled in a stepwise manner to produce a chimeric molecule with a 5′ PNA segment and a 3′ DNA segment (Finn et al., Nucleic Acids Res. 24:3357-63, 1996). Alternatively, chimeric molecules can be synthesized with a 5′ DNA segment and a 3′ PNA segment (Peterser et al., Bioorganic Med. Chem. Lett. 5:1119-11124, 1975).

In some embodiments, the therapeutic agent is a peptide nucleic acid targeting miR-155 (PNA-155).

In some embodiment, the therapeutic agent is a microRNA. As used herein, the term “miRNAs or miRs” refer to a class of high-conserved, small (about 22 nucleotides in length), single-stranded noncoding RNAs. They can bind with 3′-untranslated regions (UTRs) of mRNAs to inhibit mRNA translation or induce mRNA degradation, thus silencing gene expression at the post-transcription level. A single miRNA may regulate hundreds of target mRNAs which possess same short recognition region, simultaneously, the 3′-UTR of most mRNAs exist more than one binding site for different miRNAs. miRNAs have been reported to control the expression of approximately 30% human essential genes which are mostly essential for normal survival and development. The functions of miRNAs depend on what pathological type and physiological environment they are in, may as tumor suppressors to inhibit tumor cell proliferation, or as oncogenes to induce tumorigenesis.

In some embodiments, the therapeutic agent is a miRNA mimic. As used herein, the term “miRNA mimics” refer to chemically synthesized, double-stranded miRNA-like RNAs which are designed to copy the functionality of mature endogenous miRNA upon transfection. Similarly to miRNA, miRNA mimics bind to the 3′UTR of genes to knock down native gene expression in cells. They can be used for functionality assessments and serve as useful exogenous tools for gain-of-function studies.

In some embodiments, the therapeutic agent is miR-34 or miR-34 mimic. miRNA-34 (miR-34) has been reported to be dysregulated in various human cancers and regarded as a tumor suppressive microRNA because of its synergistic effect with the well-known tumor suppressor p53 (Hermeking H. Nat Rev Cancer. 2012; 12(9):613-26). miR-34 family has three members, including miR-34a, miR-34b and miR-34c. These three miR-34 family members are encoded by two different transcriptional units. miR-34a is located at chromosome 1p36.22 and has an unique transcript, while miR-34b and miR-34c hold one transcript in common which located at chromosome 11q23.1. In some embodiments, the therapeutic agent is miR-34a or miR-34a mimic. In some embodiments, the therapeutic agent is miR-34b or miR-34b mimic. In some embodiments, the therapeutic agent is miR-34c or miR-34c mimic.

In some embodiments, the therapeutic agent is miR-24 or miR-24 mimic. microRNA-24 (miR-24) has been shown to be associated with human cancer. The human miR-24 is located at chromosome 19 of the human genome and transcribed as a part of miR-23a-27a-24-2 cluster (Chhabra R, Mol Cancer. (2010) 9:232. doi: 10.1186/1476-4598-9-232). Dysregulation of miR-24 has been reported in various human cancers, such as non-small cell lung cancer, hepatocellular carcinoma, breast cancer, nasopharyngeal carcinoma, colorectal cancer, laryngeal squamous cell carcinoma, and esophageal squamous cell carcinoma.

In some embodiments, the therapeutic agent is miR-16 or miR-16 mimic. miR-16 is one of the first miRNAs to be linked to human malignancies (Calin G A, et al. Proc Natl Acad Sci USA. 2002, 99: 15524-15529). Evidence indicates that miR-16 can modulate the cell cycle, inhibit cell proliferation, promote cell apoptosis and suppress tumorigenicity both in vitro and in vivo. The nanoparticles described herein may also comprise at least one (e.g., two, three, or four) targeting peptide covalently-linked to the nanoparticle. Targeting peptides can be used to deliver an agent (e.g., any of the nanoparticles described herein) to a specific cell type or tissue. Targeting peptides often contain an amino acid sequence that is recognized by a molecule present on the surface of a cell (e.g., a cell type present in a target tissue). Any known targeting peptides may be used for the nanoparticles of the invention.

A variety of different methods can be used to covalently link a targeting peptide to a nanoparticle. Non-limiting examples of methods of covalently linking a targeting peptide to a nanoparticle are described in Hofmann et al., Proc. Nat. Acad. Sci. U.S.A. 10:3516-3518, 2007; Chan et al., PLoS ONE 2(11): e1164, 2007; U.S. Pat. No. 7,125,669; U.S. Patent Application Publication No. 20080058224; U.S. Patent Application Publication No. 20090275066; and Mateo et al., Nature Protocols 2:1022-1033, 2007 (each of which are incorporated by reference in their entirety). In some embodiments, the nanoparticle can be activated for attachment with a targeting peptide, for example in non-limiting embodiments, the nanoparticle can be epoxy-activated, carboxyl-activated, iodoacetyl-activated, aldehyde-terminated, amine-terminated, or thiol-activated. Additional methods for covalently linking a targeting peptide to a therapeutic nanoparticle are known in the art.

### III. Preparation of Nanoparticles of the Invention

Another aspect of the present invention is directed to methods for making the nanoparticles of the invention. In particular, the inventors of the present invention surprisingly discovered that the use of acetone:dichloromethane (DCM) as a solvent mixture during the formulation process significantly improves the morphology and size distribution of the cationic polymeric nanoparticles, e.g., the PLGA-histidine-based NPs. Specifically, the cationic polymeric nanoparticles of the present invention were formulated using the double emulsion solvent evaporation method. PLGA:poly-L-histidine formulation disclosed herein relies on ionic interactions, and involves simple mixing of the poly-L-histidine and PLGA polymer. Furthermore, using different weight ratios of PLGA and poly-L-histidine, their surface charge density could be reduced without affecting its superior transfection efficiency as compared to other cationic carriers, such as polyethyleneimine (PEI) and lipofectamine, which have a higher surface charge.

Accordingly, the present invention provides, in one aspect, a method of preparing a cationic polymeric nanoparticle comprising a therapeutic agent, comprising combining a cationic histidine peptide and a poly(lactic-co-glycolic acid) (PLGA) polymer in an organic solvent to form an organic phase. In some embodiments, the method further comprises (a) dissolving the therapeutic agent in a first aqueous phase containing water; (b) combining the organic phase with the first aqueous phase; (c) subjecting the mixture of step (b) to sonication for a sufficient period of time to produce a water-in-oil emulsion; (d) combining the water-in-oil emulsion with a second aqueous phase containing polyvinyl alcohol; (e) subjecting the mixture of step (d) to sonication for a sufficient period of time to produce a water-in-oil-in-water emulsion; (f) combining the water-in-oil-in-water emulsion with a third aqueous phase containing polyvinyl alcohol; (g) allowing the organic solvent to evaporate; and/or (h) isolating the cationic polymeric nanoparticle. In some embodiments, the organic solvent comprises acetone and/or dichloromethane.

In some embodiments, the acetone and dichloromethane are present in the organic solvent at a ratio of about 1:1 to about 20:1, about 1:1 to about 30:1 about 1:1 to about 40:1, about 1:1 to about 50:1, about 1:1 to about 60:1, about 1:1 to about 80:1, about 1:1 to about 100:1, about 1:1 to about 10:1, about 2:1 to about 50:1, about 2:1 to about 40:1, about 2:1 to about 30:1, about 2:1 to about 20:1, about 2:1 to about 10:1, about 2:1 to about 15:1, or about 2:1 to about 5:1.

In some embodiments, the acetone and dichloromethane are present in the organic solvent at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 20:1, or about 50:1.

In some embodiments, the acetone and dichloromethane are present in the organic solvent at a ratio of about 2:1.

In some embodiments, the second aqueous phase comprises about 1% to about 100%, about 1% to about 75%, about 1% to about 50%, about 1% to about 25%, about 1% to about 20%, about 1% to about 15%, or about 1% to about 10% polyvinyl alcohol. In some embodiments, the second aqueous phase comprises about 1%, about 2%, about 3%, about 4%, about 5%, about 6%, about 7%, about 8%, about 9% or about 10% polyvinyl alcohol. In some embodiments, the second aqueous phase comprises about 5% polyvinyl alcohol.

In some embodiments, the third aqueous phase comprises about 0.1% to about 10%, about 0.1% to about 8%, about 0.1% to about 5%, about 0.1% to about 2.5%, about 0.1% to about 2%, about 0.1% to about 1.5%, or about 0.1% to about 1% polyvinyl alcohol. In some embodiments, the third aqueous phase comprises about 0.1%, about 0.2%, about 0.3%, about 0.4%, about 0.5%, about 0.6%, about 0.7%, about 0.8%, about 0.9% or about 1.0% polyvinyl alcohol. In some embodiments, the third aqueous phase comprises about 0.3% polyvinyl alcohol.

In some embodiments, the solubilized phase may be filtered to recover the nanoparticles. For example, ultrafiltration membranes may be used to concentrate the nanoparticle suspension and substantially eliminate organic solvent, free drug, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated.

Diafiltration may be performed using a constant volume approach, meaning the diafiltrate (cold deionized water, e.g. about 0 to about 5° C., or 0 to about 100 C.) may added to the feed suspension at the same rate as the filtrate is removed from the suspension. In some embodiments, filtering may include a first filtering using a first temperature of about 0 to about 5° C., or 0 to about 100 C., and a second temperature of about 20 to about 300 C., or 15 to about 350 C. For example, filtering may include processing about 1 to about 6 diavolumes at about 0 to about 5° C., and processing at least one diavolume (e.g. about 1 to about 3 or about 1-2 diavolumes) at about 20 to about 300 C.

After purifying and concentrating the nanoparticle suspension, the particles may be passed through one, two or more sterilizing and/or depth filters, for example, using ^{˜}0.2 m depth pre-filter.

It will be appreciated that the amounts of polymer and therapeutic agent that are used in the preparation of the formulation may differ from a final formulation. For example, some therapeutic agent may not become completely incorporated in a nanoparticle and such free therapeutic agent may be e.g. filtered away.

### IV. Pharmaceutical Composition

Nanoparticles disclosed herein may be combined with pharmaceutical acceptable carriers to form a pharmaceutical composition. As would be appreciated by one of skill in this art, the carriers may be chosen based on the route of administration, the location of the target issue, the drug being delivered, the time course of delivery of the drug, etc. The pharmaceutical compositions can be formulated in any manner known in the art with a pharmaceutically acceptable carrier (excipient), and are suitable for administration in human or non-human subjects. Such pharmaceutical compositions may be intended for therapeutic use, or prophylactic use.

“Pharmaceutically acceptable” means that the carrier must be compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfate, chelating agents such as ethylenediaminetetraacetic acid, buffers such as acetates, citrates, or phosphates, and isotonic agents such as sugars (e.g., dextrose), polyalcohols (e.g., manitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the therapeutic nanoparticles can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.). Compositions containing one or more of any of the nanoparticles described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

The pharmaceutical compositions of this invention can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, the pharmaceutical compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles of the present invention are administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). In some embodiments, therapeutically effective amount of the nanoparticles refers to an amount that treats a disease, e.g., cancer, or reduces a symptom of a disease in a subject, e.g., a human. In some embodiments, a therapeutically effective amount of the nanoparticles refers to an amount that decreases cancer cell invasion or metastasis in a subject having cancer (e.g., a human), decreases or stabilizes tumor size in a subject, decreases the rate of tumor growth in a subject, decreases the severity, frequency, and/or duration of one or more symptoms of a cancer in a subject, or decreases the number of symptoms of a cancer in a subject (e.g., as compared to a control subject having the same disease but not receiving treatment or a different treatment, or the same subject prior to treatment).

The effectiveness and dosing of the nanoparticles described herein can be determined by a health care professional using methods known in the art, as well as by the observation of one or more symptoms of a disease, e.g., cancer, in a subject. Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of the nanoparticles described herein per kilogram of the subject's weight. While these doses cover a broad range, one of ordinary skill in the art will understand that therapeutic agents, including the nanoparticles described herein, vary in their potency, and effective amounts can be determined by methods known in the art. Typically, relatively low doses are administered at first, and the attending health care professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the nanoparticles in vivo.

The pharmaceutical compositions can be included in a container, pack, or dispenser together with instructions for administration.

The present invention further provides a nanoparticle formulation comprising a cationic polymeric nanoparticle and an organic solvent, wherein the cationic polymeric nanoparticle comprises a cationic histidine peptide and a poly(lactic-co-glycolic acid) (PLGA) polymer, and wherein the organic solvent comprises acetone and/or dichloromethane.

In some embodiments, the cationic peptide comprises a poly-L-histidine peptide.

In some embodiments, the PLGA polymer and the histidine peptide are present at a ratio of about 1:1 to about 50:1, about 1:1 to about 20:1, about 1:1 to about 30:1 about 1:1 to about 40:1, about 1:1 to about 60:1, about 1:1 to about 80:1, or about 100:1.

In some embodiments, the PLGA polymer and the histidine peptide are present at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 19:1, about 20:1, about 25:1, about 30:1, about 35:1, about 40:1, about 45:1, about 49:1, about 50:1, about 60:1, about 65:1, about 70:1, about 75:1, about 80:1, about 85:1, about 90:1, about 95:1, or about 100:1.

In some embodiments, the PLGA polymer and the histidine peptide are present at a ratio of about 3:2, about 4:1, about 19:1, or about 49:1.

In some embodiments, the PLGA polymer and the histidine peptide are present at a ratio of about 49:1.

In some embodiments, the acetone and dichloromethane are present in the organic solvent at a ratio of about 1:1 to about 20:1, about 1:1 to about 30:1, about 1:1 to about 40:1, about 1:1 to about 50:1, about 1:1 to about 60:1, about 1:1 to about 80:1, about 1:1 to about 100:1, about 1:1 to about 10:1, about 2:1 to about 50:1, about 2:1 to about 40:1, about 2:1 to about 30:1, about 2:1 to about 20:1, about 2:1 to about 10:1, about 2:1 to about 15:1, or about 2:1 to about 5:1.

In some embodiments, the acetone and dichloromethane are present in the organic solvent at a ratio of about 1:1, about 2:1, about 3:1, about 3:2, about 4:1, about 4:3, about 5:1, about 5:4, about 5:3, about 5:2, about 6:1, about 6:5, about 7:1, about 7:2, about 7:3, about 7:4, about 7:5, about 7:6, about 8:1, about 8:3, about 8:5, about 8:7, about 9:1, about 9:2, about 9:4, about 9:5, about 9:7, about 9:8, about 10:1, about 10:3, about 10:4, about 10:7, about 10:9, about 15:1, about 20:1, or about 50:1.

In some embodiments, the acetone and dichloromethane are present in the organic solvent at a ratio of about 2:1.

### V. Methods of the Invention

The nanoparticles of the present invention are suitable for treating, alleviating, ameliorating, relieving, delaying onset of, inhibiting progression of, reducing severity of, and/or reducing incidence of one or more symptoms or features of a disease, disorder, and/or condition. As demonstrated in the Examples of the application, the cationic polymeric nanoparticles disclosed herein, e.g., the PLGA-histidine-based nanoparticles, are capable of delivering therapeutic agents, e.g., small molecules, e.g., paclitaxel, peptide nucleic acids (PNA), and miRNA mimic, e.g., miRNA-34a. Specifically, in vitro and in vivo assessments demonstrated that the cationic polymeric nanoparticles, e.g., the PLGA: poly-L-histidine nanoparticles, showed optimal encapsulation of small molecule-based drug paclitaxel, and PNA based nucleic acid analog targeting microRNA-155 or miRNA mimic, possess moderate surface charge density that can target the tumor, and inhibit tumor growth after systemic administration better than conventional PLGA formulations. The safety of the cationic polymeric nanoparticles, e.g., the PLGA-histidine-based nanoparticles, was also confirmed in vivo via multiple endpoints. This present invention provides essential guidance on PLGA-histidine based formulations to generate easily scalable nanoparticles of uniform size, low polydispersity index (PDI), increased cytosolic delivery, and optimal in vivo efficacy without compromising their safety profile.

Accordingly, in one aspect, the present invention provides a method of treating a disease in a subject in need thereof, comprising administering to the subject a therapeutically effective amount of the nanoparticle of the present invention, or the pharmaceutical composition of the present invention, thereby treating the disease in the subject in need thereof. In some embodiments, the disease is cancer. In some embodiments, the disease is an autoimmune disease.

In another aspect, the present invention provides a method of reducing a tumor growth in a subject in need thereof, comprising administering to the subject a therapeutically effective amount of the nanoparticle of the present invention, or the pharmaceutical composition of the present invention, thereby reducing the tumor growth in the subject in need thereof.

In one aspect, the present invention provides a method of increasing uptake of a therapeutic agent by a cell in a subject in need thereof, comprising administering to the subject a therapeutically effective amount of the nanoparticle of the present invention, or the pharmaceutical composition of the present invention, thereby increasing uptake of the therapeutic agent by the cell in the subject in need thereof. In some embodiments, the cell is a tumor cell.

The foregoing methods can be used to treat a disease or disorder. Such disorders include, but are not limited to, cancer. Administration of the nanoparticles of the present invention can be used, for example, to reduce a disease symptom, reduce tumor size, and/or prolong survival, e.g., overall survival, and/or progression-free survival, of a subject having cancer.

As described herein, the term “cancer” refers to one of a group of diseases caused by the uncontrolled, abnormal proliferation of cells that can spread to adjoining tissues or other parts of the body. Cancer cells can form a solid tumor, in which the cancer cells are massed together, or exist as dispersed cells, as in leukemia. Types of cancer that are suitable to be treated by the nanoparticles of the present invention include, but are not limited to, solid tumors and/or hematological cancers. In one embodiment, the cancer is of epithelial origin. Exemplary types of cancer that can be treated by the foregoing methods include, but are not limited to, adrenal cancer, anal cancer, bile duct cancer, bladder cancer, bone cancer, brain/CNS tumors, breast cancer, castleman disease, cervical cancer, colon/rectum cancer, endometrial cancer, esophagus cancer, eye cancer, gallbladder cancer, gastrointestinal cancer, kidney cancer, laryngeal and hypopharyngeal cancer, leukemia, liver cancer, lung cancer, lymphoma, lymphoma of the skin, malignant mesothelioma, multiple myeloma, myelodysplastic syndrome, nasal cavity and paranasal sinus cancer, nasopharyngeal cancer, neuroblastoma, non-hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, waldenstrom macroglobulinemia, and Wilms tumor. In some embodiments, the cancer is selected from the group consisting of brain cancer, lung cancer, pancreatic cancer, melanoma, breast cancer, ovarian cancer, renal cell carcinoma, rectal adenocarcinoma, hepatocellular carcinoma, and Ewing sarcoma.

Cancer can be associated with a variety of physical symptoms. Symptoms of cancer generally depend on the type and location of the tumor. For example, lung cancer can cause coughing, shortness of breath, and chest pain, while colon cancer often causes diarrhea, constipation, and blood in the stool. Exemplary symptoms that are often generally associated with many cancers include, but are not limited to, fever, chills, night sweats, cough, dyspnea, weight loss, loss of appetite, anorexia, nausea, vomiting, diarrhea, anemia, jaundice, hepatomegaly, hemoptysis, fatigue, malaise, cognitive dysfunction, depression, hormonal disturbances, neutropenia, pain, non-healing sores, enlarged lymph nodes, peripheral neuropathy, and sexual dysfunction.

In some embodiments, the nanoparticles of the present invention can be used to inhibit the growth of cancer cells or reduce the tumor size, for example, slowing down the rate of cancer cell proliferation and/or migration, arresting cancer cell proliferation and/or migration, or killing cancer cells, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Preferably, such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by arrest of cancer cells in a particular phase of the cell cycle, e.g., arrest at the G2/M phase of the cell cycle. Inhibition of cancer cell growth can also be evidenced by direct or indirect measurement of cancer cell or tumor size. In human cancer patients, such measurements generally are made using well known imaging methods such as magnetic resonance imaging, computerized axial tomography and X-rays. Cancer cell growth can also be determined indirectly, such as by determining the levels of circulating carcinoembryonic antigen, prostate specific antigen or other cancer-specific antigens that are correlated with cancer cell growth Inhibition of cancer growth is also generally correlated with prolonged survival and/or increased health and well-being of the subject.

Nanoparticles and pharmaceutical compositions described herein are suitable for administration in human or non-human subjects. In some embodiments, the subjects are healthy individuals. In some embodiments, subjects have an existing disease, e.g., cancer. In some embodiments, suitable subjects are at risk of developing a disease, e.g., cancer. In some embodiments, suitable subjects are those who have previously had a surgery to remove tumor tissues. In some embodiments, suitable subjects are those on a therapy comprising another therapeutic agent to treat a disease, e.g., cancer, however, these therapies may be associated with adverse effects or high recurrence rates.

In some embodiments, such medicament is suitable for administration in a pediatric population, an adult population, and/or an elderly population.

The pediatric population suitable for receiving the nanoparticles of the present invention may range between 0 and 6 months of age, between 0 and 12 months of age, between 0 and 18 months of age, between 0 and 24 months of age, between 0 and 36 months of age, between 0 and 72 months of age, between 6 and 36 months of age, between 6 and 36 months of age, between 6 and 72 months of age, between 12 and 36 months of age, between 12 and 72 months of age. In some embodiments, the pediatric population suitable for receiving the nanoparticles of the present invention may range between 0 and 6 years of age, between 0 and 12 years of age, between 3 and 12 years of age, between 0 and 17 years of age. In some embodiments, the population has an age of at least 5 years, e.g., 6, 7, 8, 9, 10, 11, 12, 13, 14, 15, 16, or 17 years. In some embodiments, the pediatric population may be aged below 18 years old. In some embodiments, the pediatric population may be (a) at least 5 years of age and (b) below 18 years of age.

The adult population suitable for receiving the nanoparticles of the present invention may have an age of at least 18 years, e.g., at least 19, 20, 25, 30, 35, 40, 45, 50, 55, 60 or 65 years. In some embodiments, the adult population may be below 65 years of age. In some embodiments, the adult population may of (a) at least 18 years of age and (b) below 65 years of age. The elderly population suitable for receiving the nanoparticles of the present invention may have an age of 65 years or older (i.e., >65 years old), e.g., at least 70, 75 or 80 years.

A human subject who is likely to benefit from the treatment may be a human patient having, at risk of developing, or suspected of having a disease, e.g., cancer. A subject having cancer can be identified by routine medical examination, e.g., laboratory tests, biopsy, imaging tests, e.g., CT scans, MRI, or ultrasounds. A subject suspected of having any of such disease/disorder might show one or more symptoms of the disease/disorder. A subject at risk for the disease/disorder can be a subject having one or more of the risk factors for that disease/disorder.

A control subject, as described herein, is a subject who provides an appropriate reference for evaluating the effects of a particular treatment or intervention of a test subject or subject. Control subjects can be of similar age, race, gender, weight, height, and/or other features, or any combination thereof, to the test subjects.

The particular dosage regimen, e.g., dose, timing and repetition, used in the method described herein will depend on the particular subject and that subject's medical history.

“An effective amount” as used herein refers to the amount of each active agent required to confer a therapeutic effect on the subject, either alone or in combination with one or more other active agents. For example, an effective amount refers to the amount of the nanoparticles of the present invention which is sufficient to achieve a biological effect, e.g., a reduction of tumor size.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting.

The present invention is further illustrated by the following examples, which are not intended to be limiting in any way. The entire contents of all references, patents and published patent applications cited throughout this application, as well as the Figures, are hereby incorporated herein by reference.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1: Formulation, Characterization, and Cellular Uptake of PLGA-L-Histidine Nanoparticles

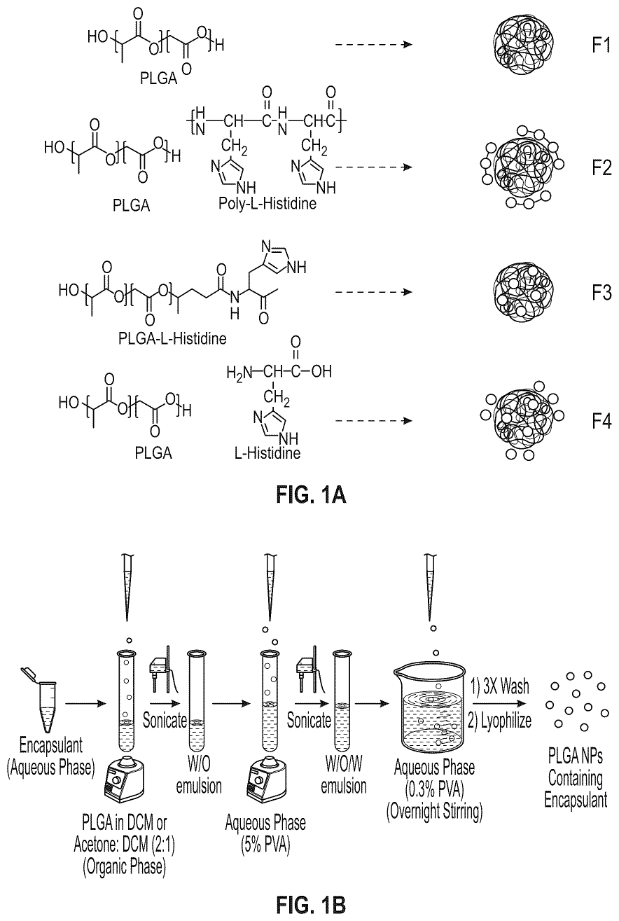

To produce effective and stable histidine-containing PLGA NP formulations, three blank and coumarin dye (C6) containing PLGA-histidine formulations were tested (FIG. 1A): PLGA polymer (1) in combination with poly-L-histidine peptide (F2) at a ratio of PLGA:poly-L-histidine, 4:1, (2) in combination with unconjugated histidine amino acid (F4), and (3) covalently conjugated to monomeric histidine units (F3). Also formulated were a blank and C6 containing regular PLGA formulation as a control for the study (F1). L-histidine was chosen herein, as it is a naturally occurring essential amino acid and does not render any toxicity (Moro et al. Nutrients 2020). Also, the endosomal escape is a crucial step for the intracellular delivery of NP-based delivery systems. Due to the imidazole ring, histidine exerts the proton sponge effect-based endosomolytic properties, which can increase the cytoplasmic distribution of encapsulant (Chen et al. Nucleic Acids Res. 2002, 30 (6), 1338-45).

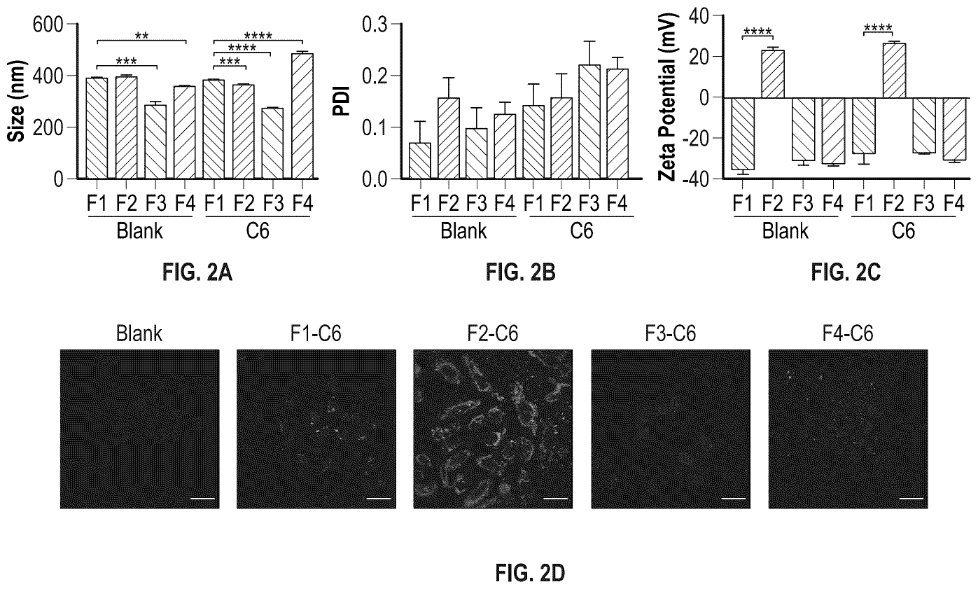

Initially, double emulsion solvent evaporation technique was used to formulate the NPs using (FIG. 1B) DCM as organic solvent. For initial studies, blank and coumarin dye (C6) containing NP formulations were formulated. A significant change in the size and distribution was not observed, as all the NPs showed consistent hydrodynamic size (290-410 nm) and size distribution (0.12-0.22) (FIG. 2A and FIG. 2B). However, zeta potential analysis showed that F2 formulation exhibit a positive surface charge density (+22 mV), whereas other formulations have a negative surface charge density near −40 mV (FIG. 2C). These results indicated that the positive zeta potential of F2 formulations could be due to the presence of poly-L-histidine units on PLGA NP formulations' surface. Further, a confocal microscopy-based assessment was performed to compare the transfection efficiency of the aforementioned formulations.

HeLa cells were treated with NPs containing C6 (indicated as F-C6) for 24 hours, followed by confocal microscopy analysis as shown in FIG. 2D. A moderate cytoplasmic distribution of C6 containing NPs was observed for F1, F3, and F4 formulations. In contrast, the F2 formulation demonstrated higher cellular uptake and uniform distribution across the cytoplasm. One plausible explanation of these discerning results is the positive surface charge density of F2 formulation which increases its cellular uptake efficiency due to higher interaction with the cell membrane.

### Example 2. Nanoparticle Optimization Using Acetone:Dichloromethane as a Solvent System

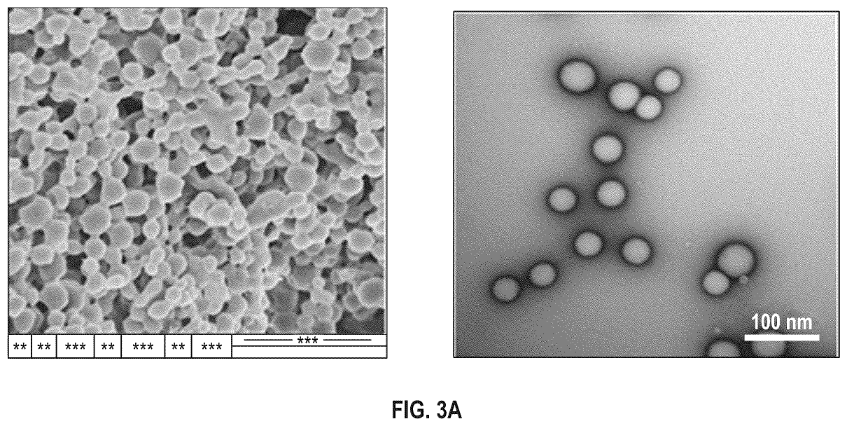

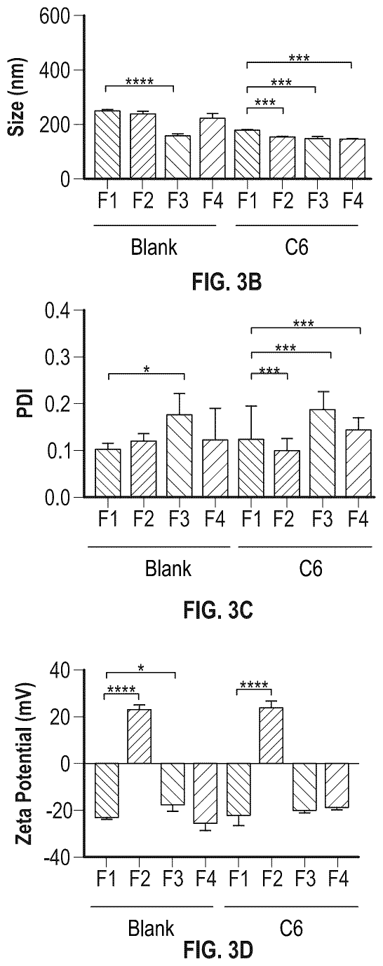

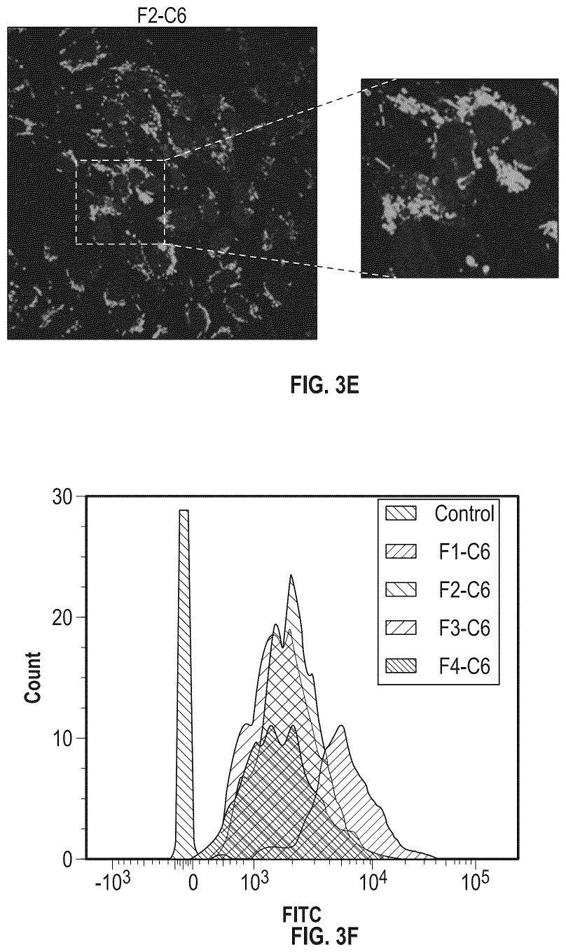

Next, NP formulations were formulated containing C6 using acetone:DCM (2:1) solvent mixture instead of DCM in double emulsion solvent evaporation-based protocol. For NPs generated by acetone:DCM solvent mixture result in uniform morphology as indicated by transmission electron microscopy (TEM) and scanning electron microscopy (SEM) images (FIG. 3A). The NPs formulated with acetone:DCM as the organic solvent mixture possessed a smaller size (170-200 nm in diameter) and superior uniform size distribution with PDIs of 0.10-0.18 as compared to NPs generated using DCM as an organic solvent (FIG. 3B and FIG. 3C). In contrast, however, a minimal change in zeta potential values (FIG. 3D) were observed between the two NPs, indicating that the histidine arrangement in NP formulation (or surface) does not change when acetone:DCM is used as a solvent mixture during the formulation process. Further, cellular uptake studies were performed with NPs generated using acetone:DCM solvent mixture. Consistent with prior findings, uniform cellular uptake of C6 containing F2 NPs formulated using acetone:DCM mixture (FIG. 3E) was confirmed. Further, quantitative assessment was performed using flow cytometry-based assays.

As shown in FIG. 3F, the FITC intensity in the HeLa cells treated with F2 NPs containing C6 was higher than the control, F1, F3, and F4 formulations. These results confirmed that F2 formulations undergo substantial cellular uptake compared to other formulations. Subsequent studies were centered on F2 NPs formulated using acetone:DCM solvent mixture and double emulsion solvent evaporation method.

### Example 3. Optimization of PLGA: Poly-L-Histidine Ratio and their Characterizations

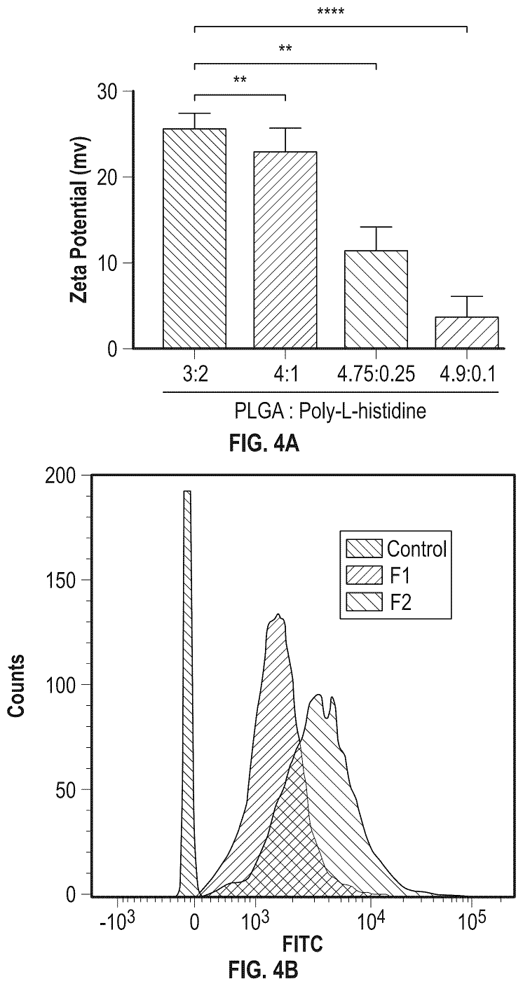

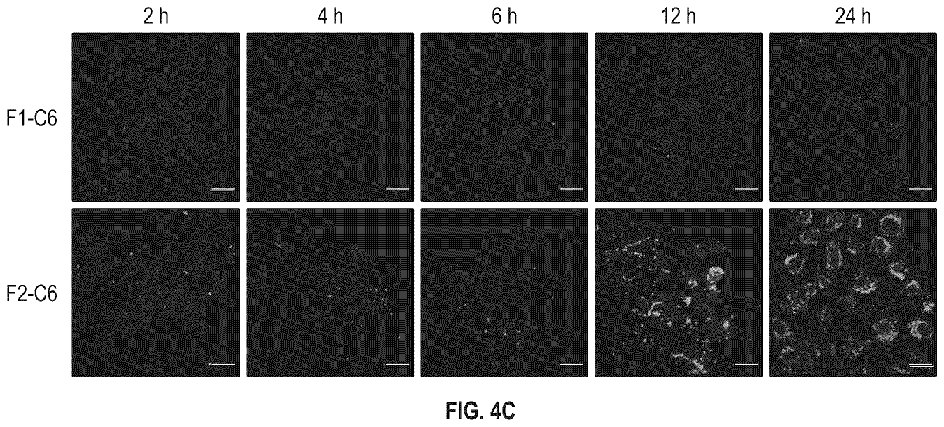

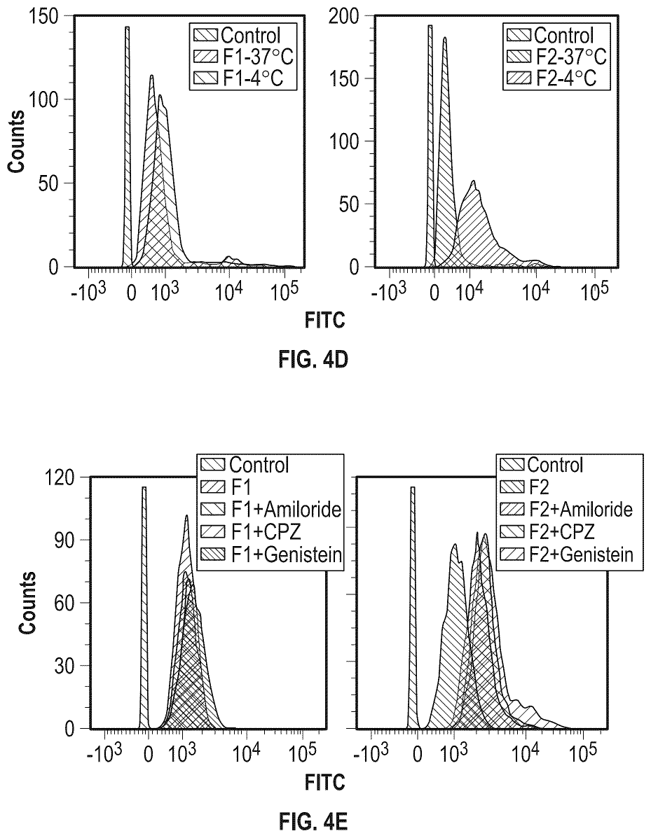

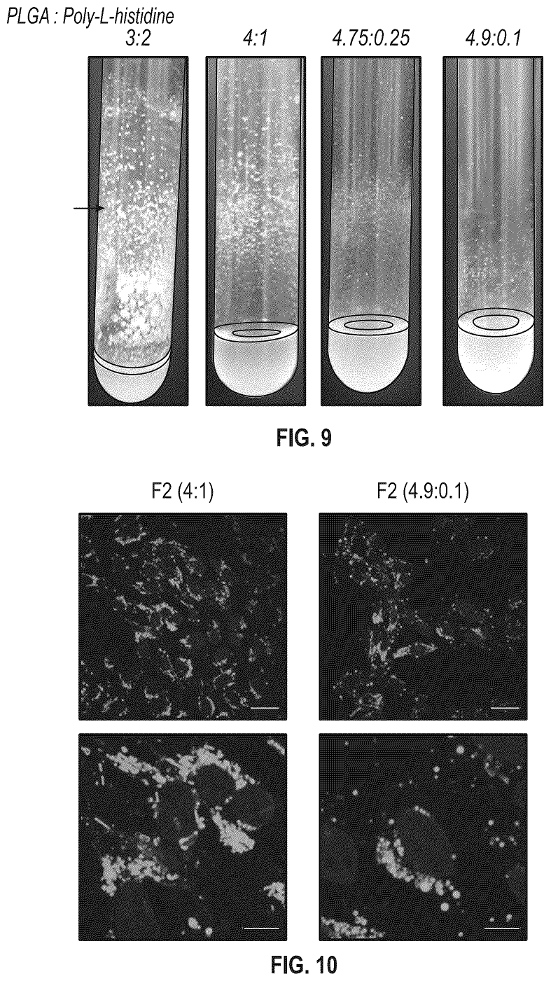

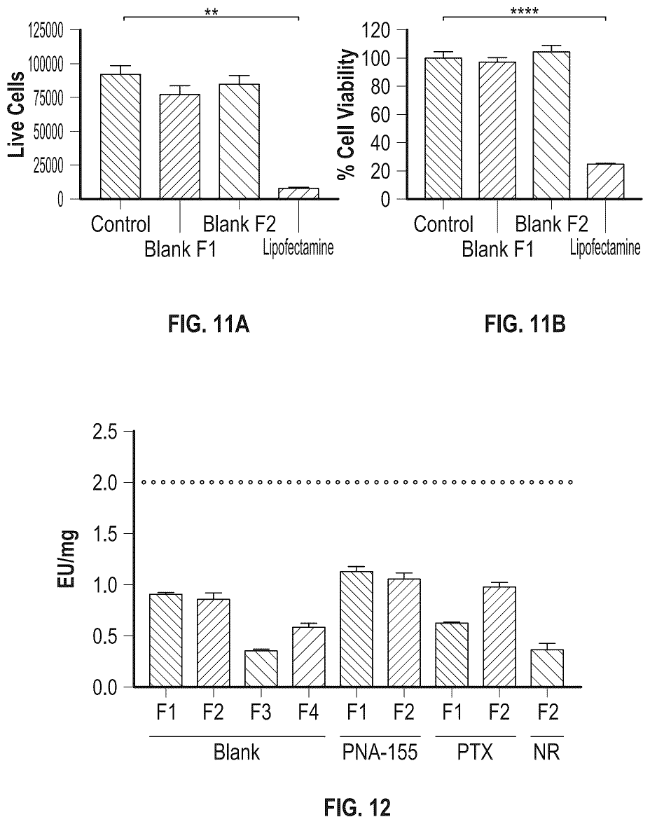

The initial F2 formulation based on PLGA:poly-L-histidine stoichiometric ratio of 4:1 showed a zeta potential of +22 mV. Prior studies indicated that cationic NP formulations based on PBAE or PEI showed significant toxicity due to the high cationic surface charge density (zeta potential of +25 mV) (Little et al. J Control release 2005, 107, 449-62; Omidi et al. BioImpacts 2011, 1 (1), 23-30). Hence, an effort was made to reduce the F2 NP formulations' positive surface charge without affecting its transfection efficiency. Comprehensive physicochemical characterization of NPs formulated with different stoichiometric ratios of PLGA were performed: poly-L-histidine (3:2, 4:1, 4.75:0.25, and 4.9:0.1). an increase in ionic precipitate formation was found by increasing the poly-L-histidine content during the formulation (FIG. 9) due to the higher ionic interaction between PLGA and poly-L-histidine units. As indicated by zeta potential results, a decrease in surface charge density was observed, as the poly-L-histidine amount was reduced in the F2 formulation (FIG. 4A). It has been well established that a lower cationic surface charge reduces the cellular and systemic toxicity of NPs (Fröhlich et al. Int. J. Nanomedicine 2012, 7, 5577-91). Hence, PLGA:poly-L-histidine at a ratio of 4.9:0.1 with an optimal positive charge density (+5 mV) was chosen for later studies. To ensure that a decrease in cationic charge does not impact the transfection efficiency of F2 formulation, confocal microscopy-based assessment was performed. A significant cytoplasmic distribution was observed with F2 formulation containing a 4.9:0.1 ratio indicated by C6 fluorescence intensity (FIG. 10). Furthermore, the cellular uptake results were validated by flow cytometry analysis (FIG. 4B) where F2 formulation comprising of PLGA:poly-L-histidine ratio of 4.9:0.1 showed higher cellular uptake as compared to conventional F1 formulation. Next, kinetics of cellular uptake with F2 and F1 formulations at different time points (i.e., 2, 4, 6, 12, and 24 hours) were also investigated followed by confocal imaging. Cellular uptake increased after ˜12 hours of incubation of HeLa cells with F2 NPs compared to F1 NPs (FIG. 4C). Hence, the F2 formulation containing a PLGA:poly-L-histidine stoichiometric ratio of 4.9:0.1 was used for following studies. Further, the safety of NPs in two primary cell lines, HEK293 and PBMC, were tested. The cell viability of blank, F1, and F2 NPs treated cells were compared to lipofectamine-treated cells. Lipofectamine is a well-established cationic lipid-based approach for the transfection of nucleic acids at equivalent doses (Cardarelli et al. Sci. Rep. 2016, 6 (May), 1-8). A significant reduction in cell viability was observed after treatment with lipofectamine in both PBMC and HEK cells. Whereas F2 NPs did not affect the viability of the cells as compared to control samples indicating the safety of F2 NPs (FIG. 11). The present formulations were also tested for endotoxin levels using limulus amebocyte lysate (LAL) chromogenic endotoxin quantification assay. The permissible endotoxin limit was calculated based on the NP dose used for preclinical evaluations in mice models (Malyala et al. J. Pharm. Sci. 2008, 97 (6), 2041-44). All the present formulations exhibited lower endotoxin levels than the permissible limit indicating safety and sterility of the present formulations (FIG. 12).

### Example 4. Mechanism of Cellular Uptake of PLGA: Poly-L-Histidine NPs

Inclusive studies were performed to decipher the mechanism of cellular uptake of F2 formulation. First, to examine the role of endocytosis in the uptake of F2 formulation, temperature-dependent cell uptake studies were performed in the HeLa cells at physiological (37° C.) and low temperature (4° C.) by flow cytometry-based analysis. It is well ascertained that low temperature decreases the endocytosis driven transport across the cell membranes (Goldenthal et al. Exp. Cell Res. 1984, 152 (2), 558-64). Flow cytometry results confirmed that the cytoplasmic delivery of F2 formulation decreases substantially at 4° C. than conventional F1 formulation (FIG. 4D). These results indicated that endocytosis plays an essential role in the cellular transport of F2 formulations composed of PLGA:poly-L-histidine.

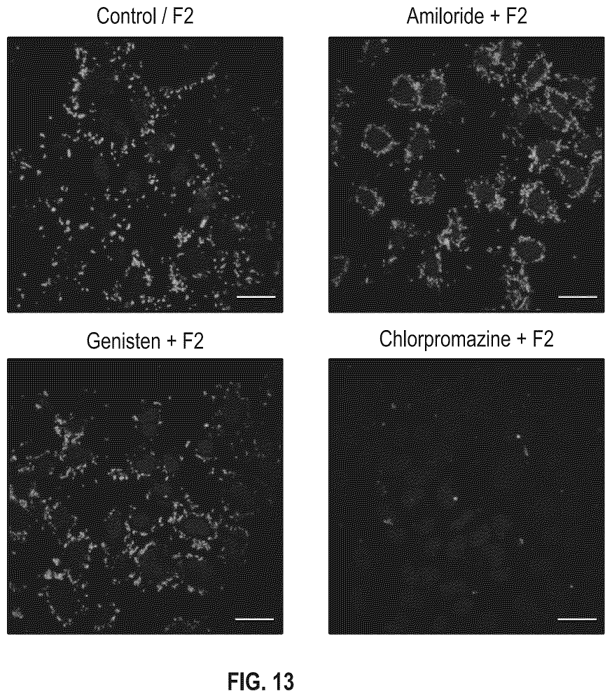

Further, the cellular uptake of F2 NPs was investigated in the presence of different endocytosis inhibitors to identify the endocytic pathway contributing towards the higher transfection of F2 NPs. Several prior studies established that chlorpromazine (CPZ) prevents clathrin (Wang et al. J. Cell Biol. 1993, 123 (5), 1107-17), genistein inhibits caveolae (Nabi et al. J. Cell Biol. 2003, 161 (4), 673-77), and amiloride blocks the macropinocytosis mediated endocytosis across the cell membrane (Koivusalo et al. J. Cell Biol. 2010, 188 (4), 547-63). Endocytosis inhibitor studies were performed by incubating the HeLa cells with indicated inhibitor and F2 formulation for 2 hours to ensure the optimal cell viability. Flow cytometry results indicated substantial decrease in cell uptake of F2 NPs in HeLa when co-incubated with CPZ, however genistein and amiloride showed minimal change in uptake of F2 NPs in HeLa cells (FIG. 4E). Further, the role of clathrin-mediated endocytosis in the cellular uptake of F2 formulation by confocal microscopy also was confirmed (FIG. 13). These results established the role of clathrin mediated endocytosis in the cellular uptake of F2 formulation.

### Example 5. Small Angle Neutron Scattering (SANS) Analysis

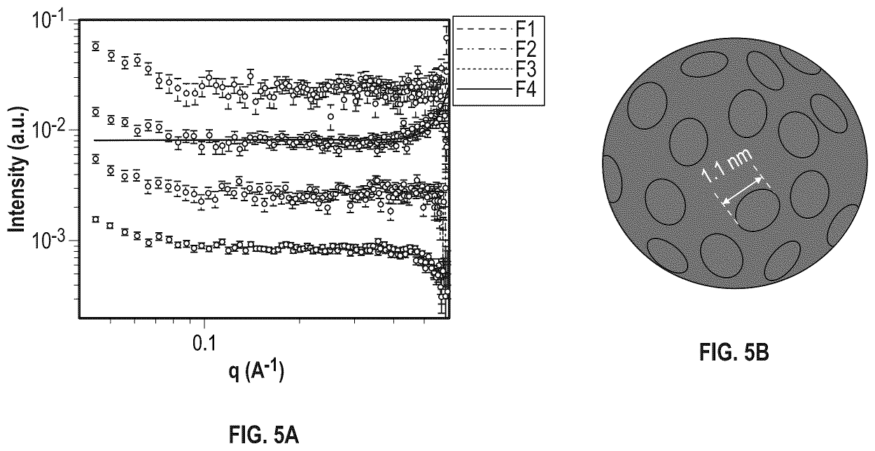

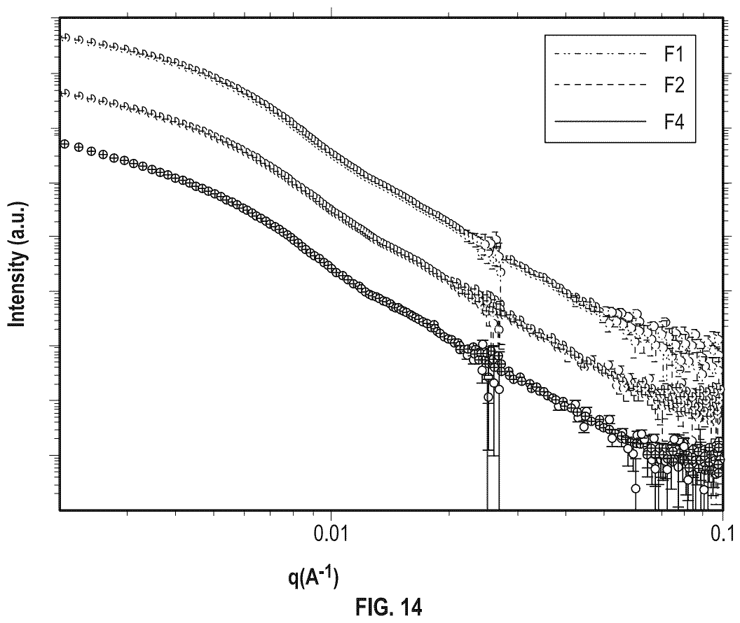

The SANS data of samples in contrast matched condition revealed nearly identical and flat curves indicative of null difference below 0.4 Å⁻¹ (FIG. 5A). Interestingly, the SANS data of F2 NPs in contrast-matched conditions at a high q regime (q>0.4 Å⁻¹) shows a significant difference compared to those of F1, F2, and F4 NPs (FIG. 14). The scattering intensity increases with an increased q value. Several attempts to best fit the SANS data using form factor (describing the shape) have been made (Kline J. Appl. Crystallogr. 2006, 39 (6), 895-900).

The only feasible model to describe uprising intensity is through applying a structure factor (Percus et al. Phys. Rev. 1958, 110 (1), 1-13), suggesting a specific correlation length in F2 formulation. Further, a domain size (diameter) of 1.1 nm with a volume fraction of 70% was used to best fit the data.

Without being bound by theory, analysis of these results seems to indicate that the poly-L-histidine forms cationic domains on the NPs surface with a relatively packed spacing, as shown in FIG. 5B. The presence of poly-L-histidine domains on the F2 NPs structure is consistent with the positive surface charge density noticed in DLS studies, followed by increasing its transfection efficiency in the cell culture studies.

### Example 6. In Vitro Efficacy Studies with PNA-155 Containing F2 Formulation

To examine the efficacy of F2 formulation containing nucleic acid analogs, F2 NPs containing anti-miR-155 PNAs (or PNA-155) were formulated by double emulsion solvent evaporation-based method. PNA-155 targets oncomiR-155, which is overexpressed in numerous solid tumors (Volinia et al. Proc. Natl. Acad. Sci. U.S.A 2006, 103 (7), 2257-61) such as breast (Mattiske et al. Cancer Epidemiol. Biomarkers Prev. 2012, 21 (8), 1236-43), colon (Schetter et al. JAMA—J. Am. Med. Assoc. 2008, 299 (4), 425-36), lung (Zhang et al. Medicine (Baltimore). 2020, 99 (33), e21483) and various lymphomas including diffuse large B cell lymphoma (Kluiver et al. J. Pathol. 2005, 207 (2), 243-49) and Burkitt's lymphoma (Metzler et al. Genes Chromosom. Cancer 2004, 39 (2), 167-69). Prior studies reported that PLGA formulation can encapsulate a moderate quantity of anti-miR-155 PNA (Babar et al. Proc. Natl. Acad. Sci. U.S.A 2012, 109 (26)).

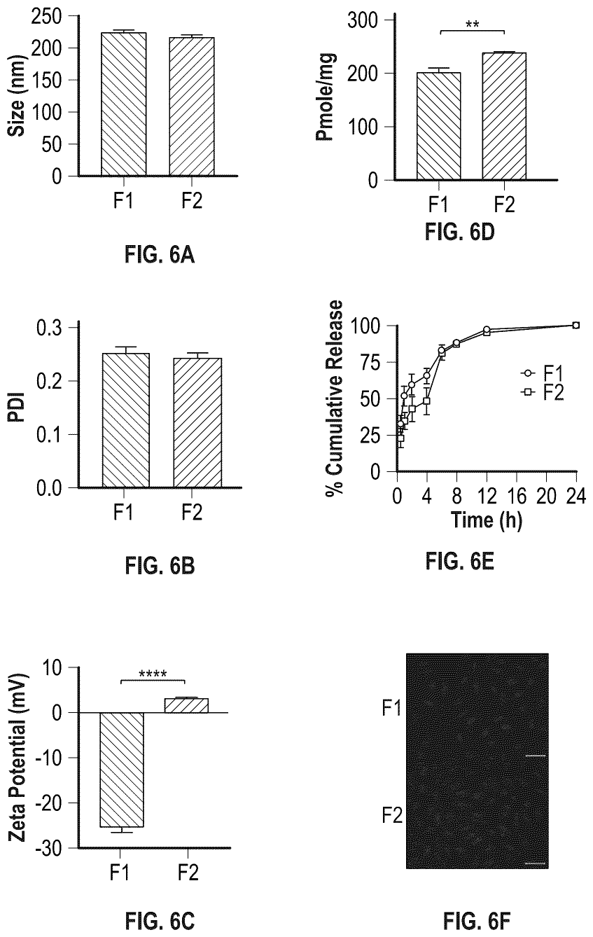

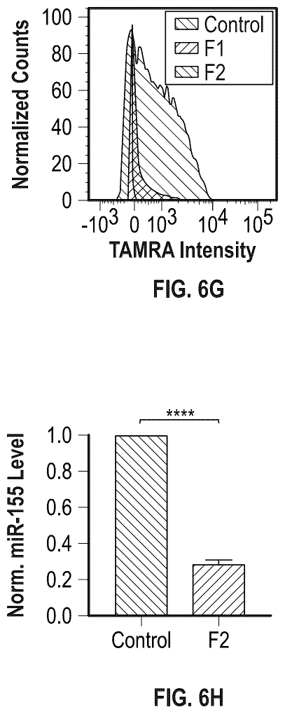

Hence, as a control, an F1 formulation containing PNA-155 also was generated. F1 and F2 formulations containing PNA-155 yielded a hydrodynamic particle size of 223-216 nm and PDIs of 0.24-0.23, respectively (FIG. 6A and FIG. 6B). Consistent with prior results, PNA-155 containing F1 formulation demonstrated negatively charged zeta potential (−25 mV), whereas F2 formulation containing PNA-155 possesses the cationic surface charge density (+3 mV) (FIG. 6C). Further, PNA-155 loading analysis was performed using the solvent extraction method followed by their UV-Vis absorbance at 260 nm.

F1 and F2 formulations showed a PNA loading of ˜208 pmoles/mg and ˜248 pmoles/mg of NPs respectively (FIG. 6D). The time-dependent release kinetics of PNA-155 in F1 and F2 formulation also was tested by incubating the NPs in PBS followed by measuring the UV-Vis absorbance at 260 nm (FIG. 6E). Both F1 and F2 NPs showed release of PNA-155 within 24 hours. Further, the transfection efficiency of F2 formulations containing PNA-155 in HeLa and U2932 cells was tested. Since U2932 cells are suspended lymphoma cells, the cellular uptake of PNA-155 F1 and F2 NPs was studied by confocal microscopy in HeLa cells which are adherent in nature. Uniform and superior intracellular uptake of F2 NPs was observed in comparison to F1 NPs in HeLa cells by confocal microscopy (FIG. 6F). Similarly, the cellular uptake of PNA-155 containing F1 and F2 formulations in U2932 cells was quantified using flow cytometry analysis (FIG. 6G). PNA-155 containing F2 formulation showed significant cellular uptake as compared to F1 formulation in both U2932 and HeLa cells.

Next, to investigate the functional activity of PNA-155, U2932 cells were treated with PNA-155 containing F2 NPs for 24 hours followed by qRT-PCR analysis. PNA-155 containing F2 formulation exhibited more than 75% knockdown of miR-155 compared to untreated U2932 cells (FIG. 6H). Overall, these results depict that higher cellular transfection of F2 formulation containing PNA-155 results in optimal efficacy.

### Example 7. In Vitro Efficacy Studies with Paclitaxel Loaded F2 NP Formulations

To test the delivery and efficacy of small molecule-based drug candidates, the paclitaxel (PTX) loaded F2 NPs were formulated. Further, physico-biochemical characterization was performed on PTX loaded F2 formulations. PTX loaded F2 formulation showed uniform size of 155 nm and PDI of 0.14 (Table 1).

TABLE 1
Characterization of PLGA nanoparticle formulations encapsulated
with paclitaxel for mean hydrodynamic size (DLS), polydispersity
index (PDI) and surface charge (zeta potential).

| PLGA | DLS Size | | Zeta potential |
|---|---|---|---|
| formulation | (nm) ± SD | PDI | (mV) ± SD |
| PTX F1 | 162.31 ± 13.31 | 0.05 ± 0.02 | −19.13 ± 1.66 |
| PTX F2 | 155.01 ± 3.40 | 0.14 ± 0.02 | 4.85 ± 1.26 |

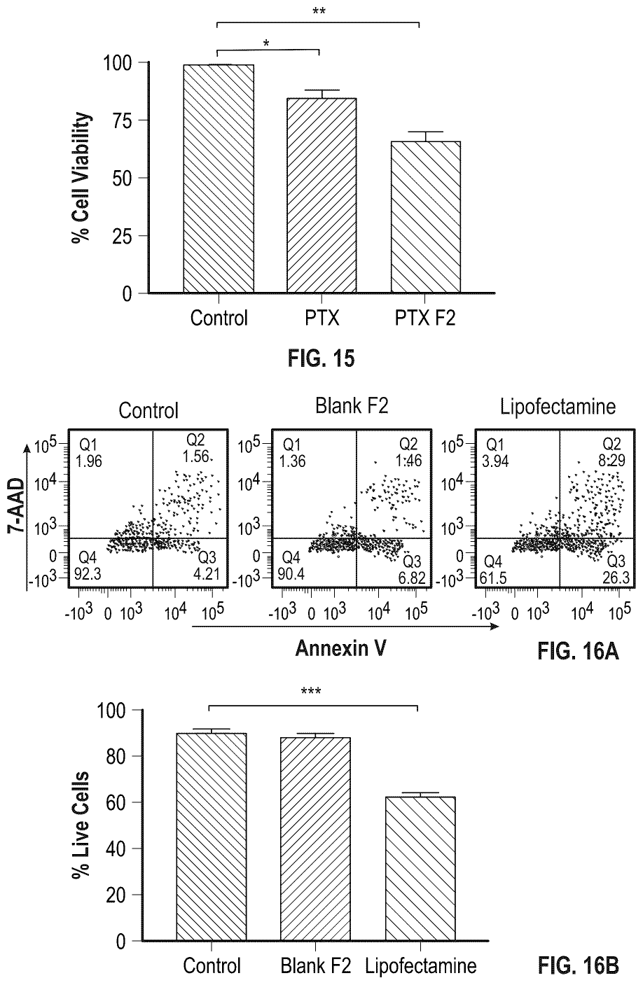

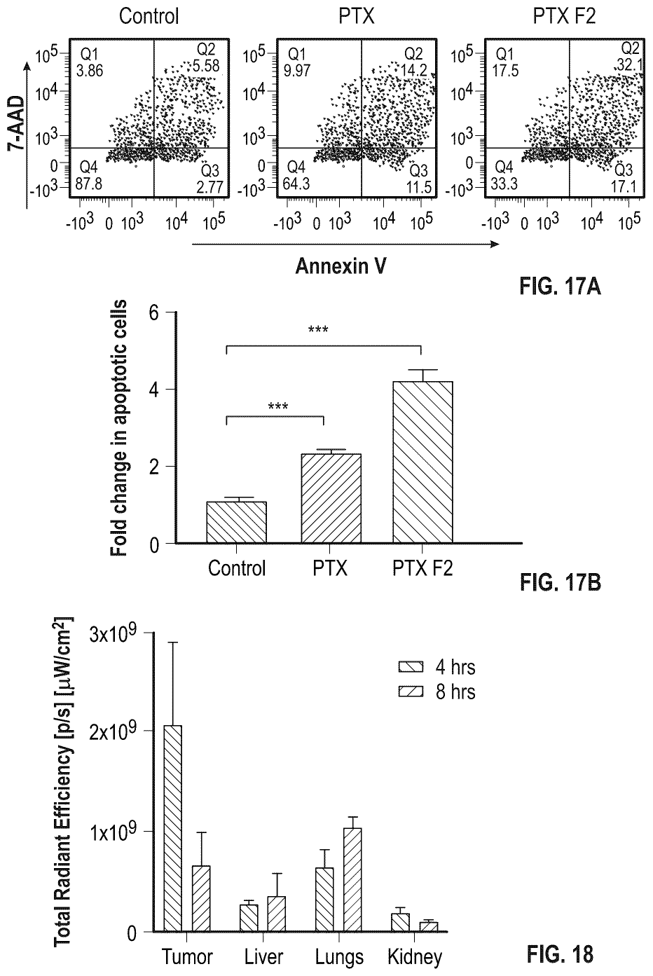

Consistent with prior results, PTX loaded F2 formulation also was observed to exhibit positive surface charge (+4.85 mV). F1 and F2 formulations showed a PTX loading of ˜1.8 ug/mg and ˜1.5 ug/mg of NPs respectively. Further, a cell proliferation assay was performed onto HeLa cells treated with PTX loaded F2 formulation compared to PTX suspension. HeLa cells treated with F2 formulations showed 25% reduction in cell viability, whereas PTX suspension-treated cells showed 17% decrease in cell viability (FIG. 15). To examine whether the decrease in HeLa cell viability is due to enhanced apoptosis, annexin based apoptotic detection assay using flow cytometry was performed. First, apoptosis analyses were performed on blank F2 NPs and lipofectamine treated HeLa cells. Blank F2 NPs did not induce apoptosis in HeLa cells. However, lipofectamine-treated cells showed a 40% decrease in viability (FIG. 16). Next, the apoptosis assay was performed on PTX F2 NPs and PTX suspension-treated HeLa cells at an equivalent dose. Consistent with cell proliferation results, F2 formulation containing PTX caused 2 and 4-fold higher apoptosis in HeLa cells than the PTX suspension treated and control samples, respectively (FIG. 17). Collectively, these results demonstrated that F2 formulation could encapsulate PTX in optimal amount and exhibit significant efficacy in cell proliferation and apoptosis-based studies.

### Example 8. In Vivo Evaluation of PNA-155 Containing F1 and F2 Formulations

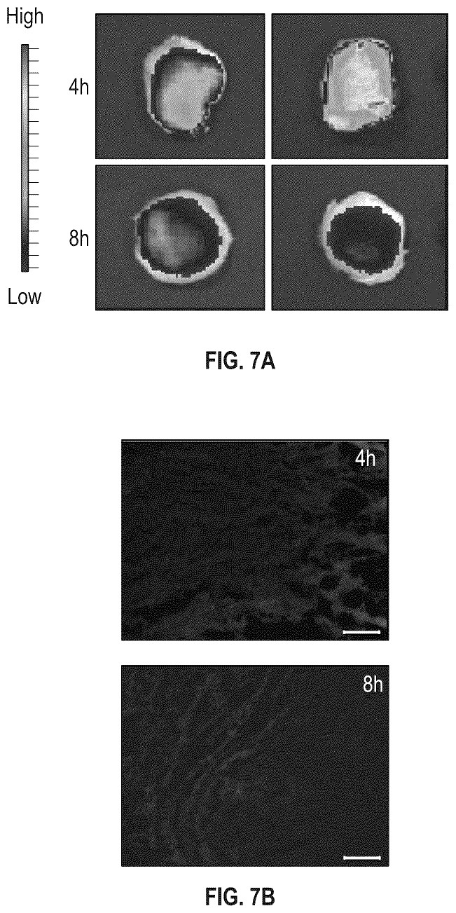

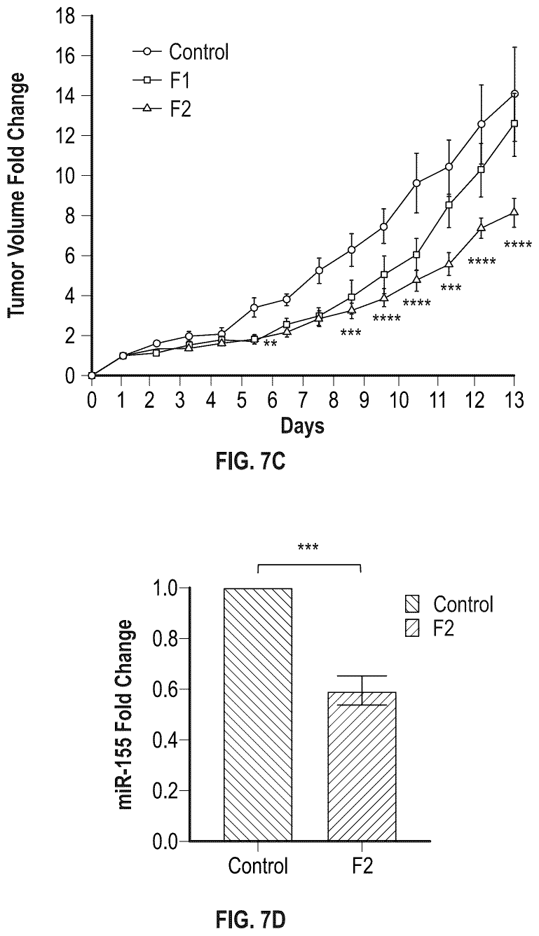

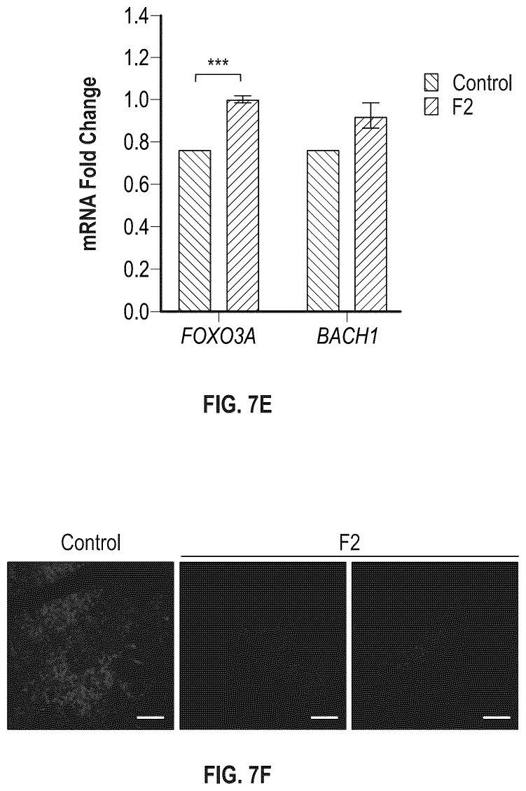

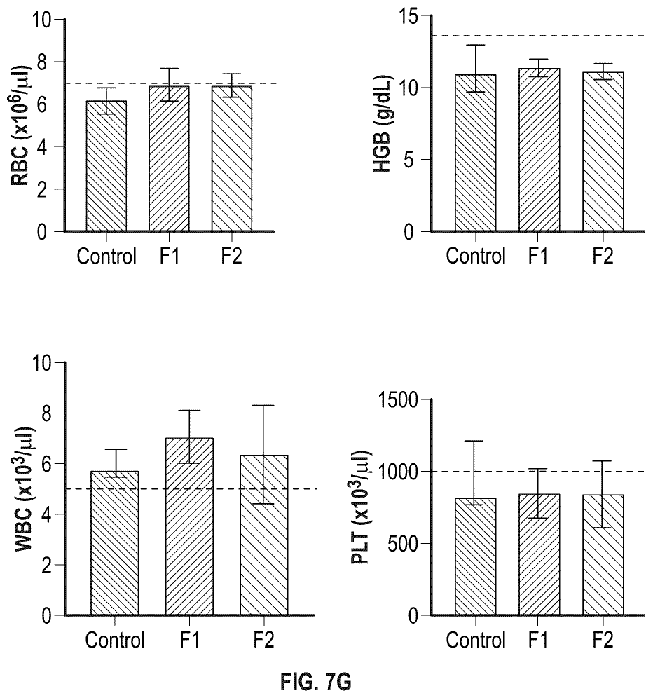

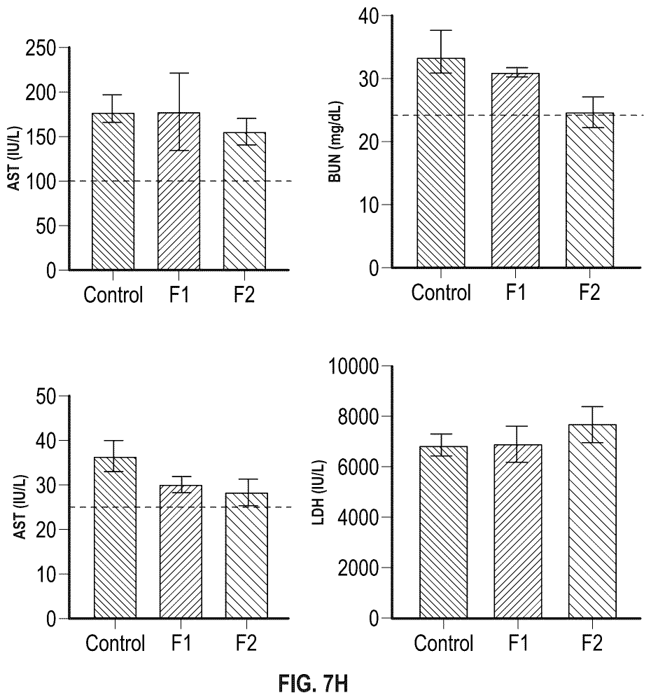

Next, the efficacy of F1 and F2 NPs containing PNA-155 was tested in U2932 derived xenograft mice model. First, the biodistribution of F2 NPs containing nile red dye was studied in the xenograft mice. F2 NPs were administered systemically in xenograft mice bearing tumors of volume ˜100-200 mm³. Mice were euthanized after 4-hours and 8-hours of systemic administration followed by collection of tumor and organs. The accumulation of F2 NPs in tumors and other organs was determined and quantified by IVIS imaging (FIG. 7A and FIG. 18). A significant accumulation of F2 NPs in tumors were observed both after 4-hours and 8-hours of systemic injection due to the enhanced permeability and retention effect (EPR) (Torchilin Adv. Drug Deliv. Rev. 2011, 63 (3), 131-35). Further, the distribution of F2 NPs in the tumor tissue was confirmed via confocal microscopy (FIG. 7B). A significant accumulation of F2 NPs was observed in the tumor sections both after 4-hours and 8-hours of systemic administration. Non-specific accumulation in kidney, heart, spleen, or bone marrow was not observed. Although, some accumulation of F2 NPs was observed in the lungs followed by liver.

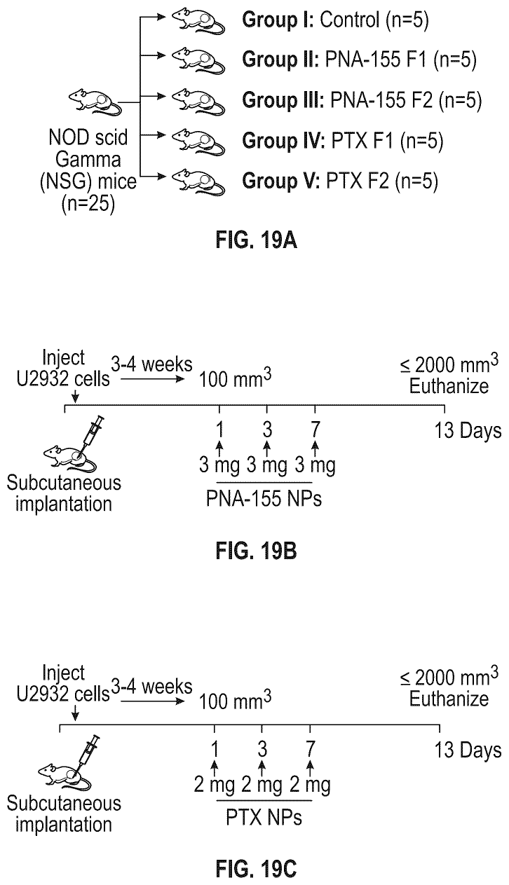

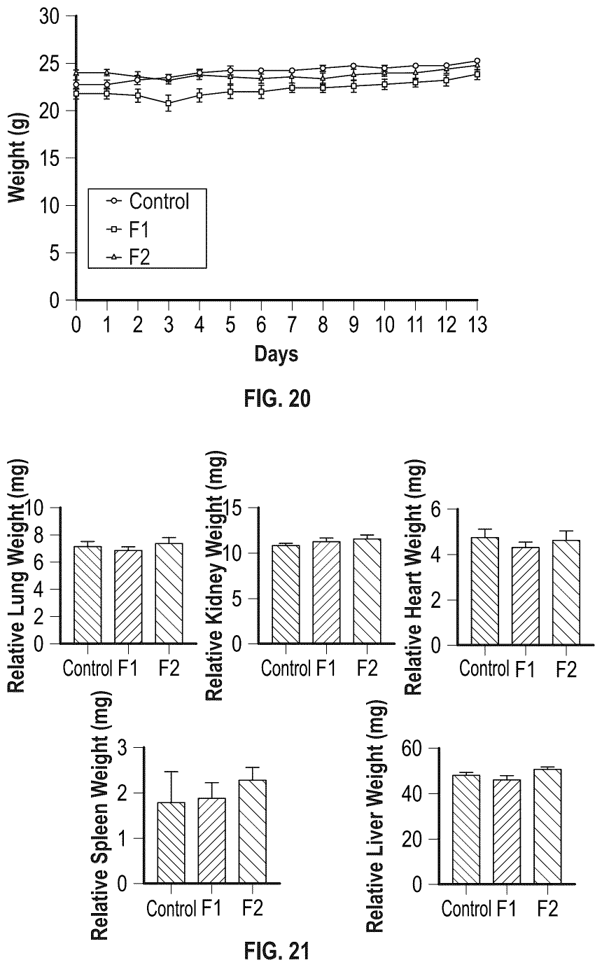

Next, the effect of F1 and F2 NPs containing PNA-155 on tumor growth in U2932 derived xenograft mice model was evaluated. Once the tumor volume reached ˜100-200 mm³, NPs were administered systemically at multiple doses for a total PNA-155 dose of 0.6 mg/kg (FIG. 19A and FIG. 19B). PNA-155 F2 NPs showed considerable delay (˜6-fold decrease) in tumor growth (FIG. 7C) relative to the control group. However, F1 NPs treated mice exhibited only ˜2-fold decrease in tumor growth. The weight of mice from each group was found to be similar during the study (FIG. 20). Further, a significant decrease (-45%) in the levels of miR-155 in F2 NPs treated tumors was observed (FIG. 7D). It has been established that FOXO3A and BACH1 are the direct downstream target genes of miR-155 (Cheng et al. Nature 2015, 518 (7537), 107-10) and hence their expression levels in the treated tumor samples was evaluated.

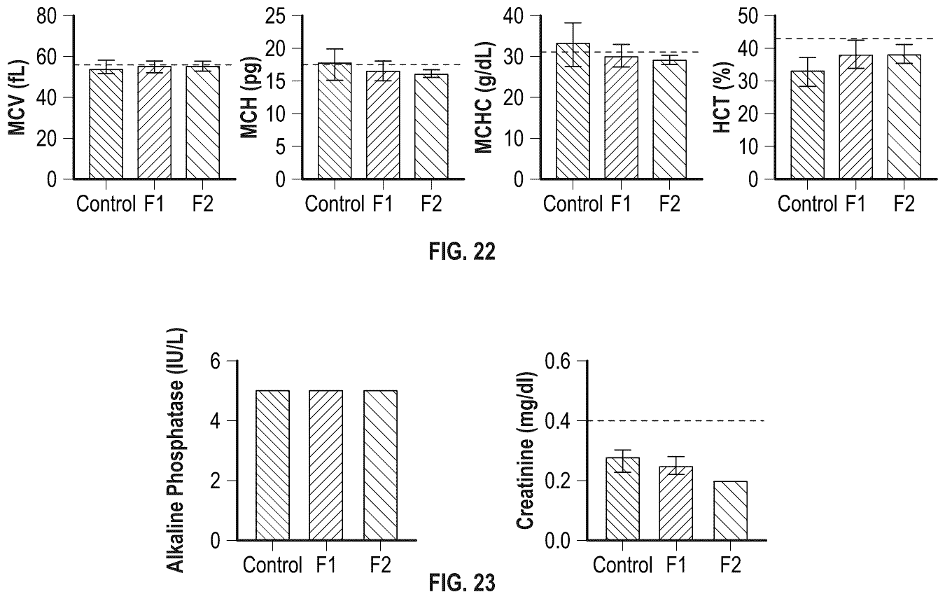

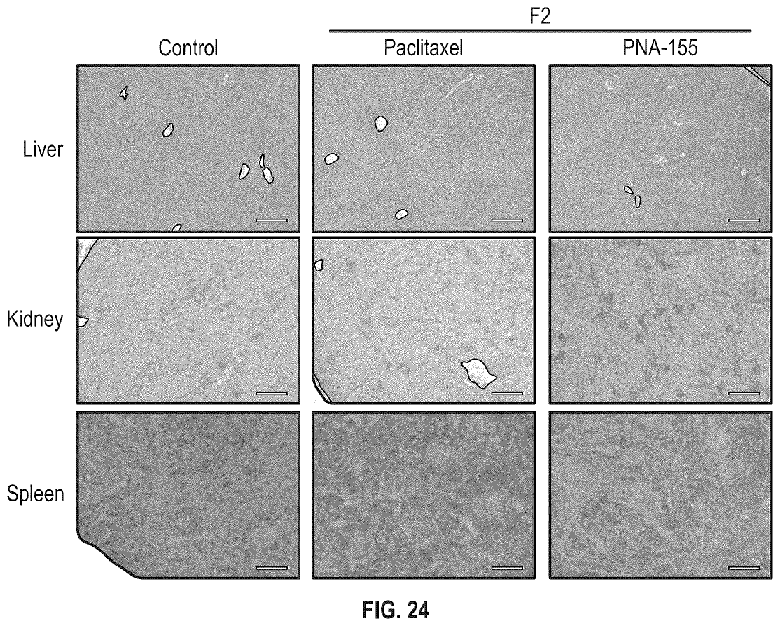

Significant upregulation of FOXO3A (˜1.3 fold) and BACH1 (˜1.2 fold) was observed in F2 NPs treated tumors compared to the control group (FIG. 7E). In addition, Ki67 staining showed lowest proliferation in F2 treated tumors (FIG. 7F). Further, comprehensive blood chemistry, complete blood count (CBC), and organ histological analysis were performed to assess the safety profile of F2 NPs. No toxic effects were observed in mice treated with PNA-155 containing F2 NPs in comparison to the control group indicating the safety of positively charged F2 formulations. The weight of major organs in both F1 and F2 NPs treated group was similar to the control group (FIG. 21). No changes were observed in complete blood count analysis (FIG. 7G and FIG. 22) and the levels of alanine aminotransferase (ALT), aspartate aminotransferase (AST), lactate dehydrogenase (LDH), blood urea nitrogen (BUN), creatinine, and alkaline phosphatase (FIG. 7H and FIG. 23). Likewise, no significant organ toxicity was observed in liver, kidney, and spleen histology in PNA-155 F2 NPs treated mice establishing their safety profile (FIG. 24).

### Example 9. In Vivo Evaluation of Paclitaxel Containing F1 and F2 Formulations

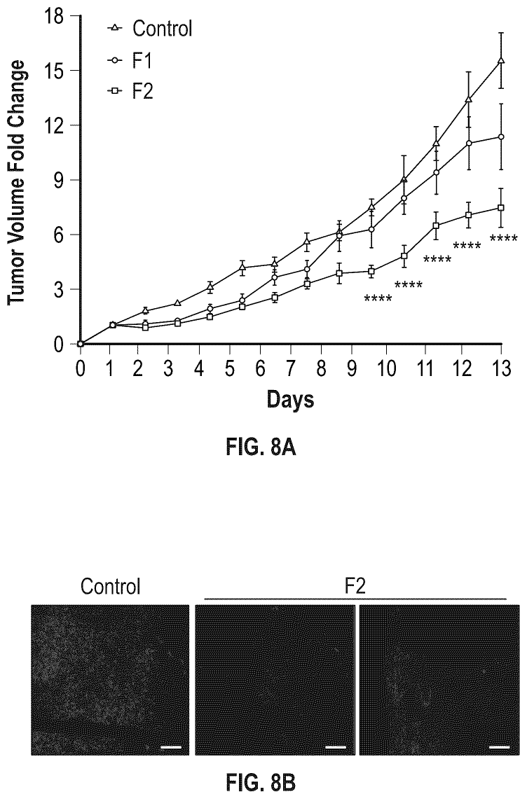

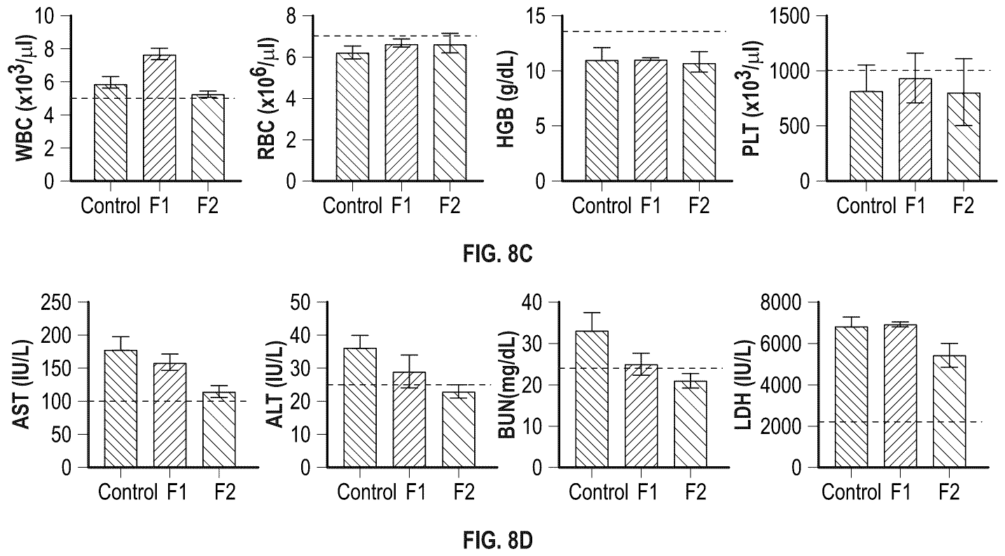

Similarly, superior efficacy of paclitaxel loaded F2 NPs was observed in comparison to the F1 NPs in the xenograft mice study. Paclitaxel NPs were administered systemically at multiple doses for a total paclitaxel dose of 0.4 mg/kg (FIG. 19C). PTX loaded F2 NPs showed significant tumor growth decrease (˜6.5 fold). However, F1 NPs showed only ˜4-fold decrease (FIG. 8A) in tumor growth. Further, significant efficacy of PTX F2 NPs was confirmed by Ki67 staining wherein PTX F2 NPs treated tumors showed reduced proliferation (FIG. 8B).

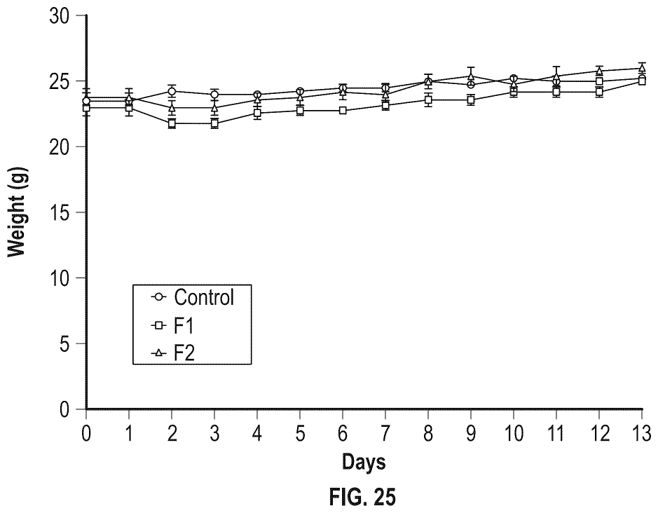

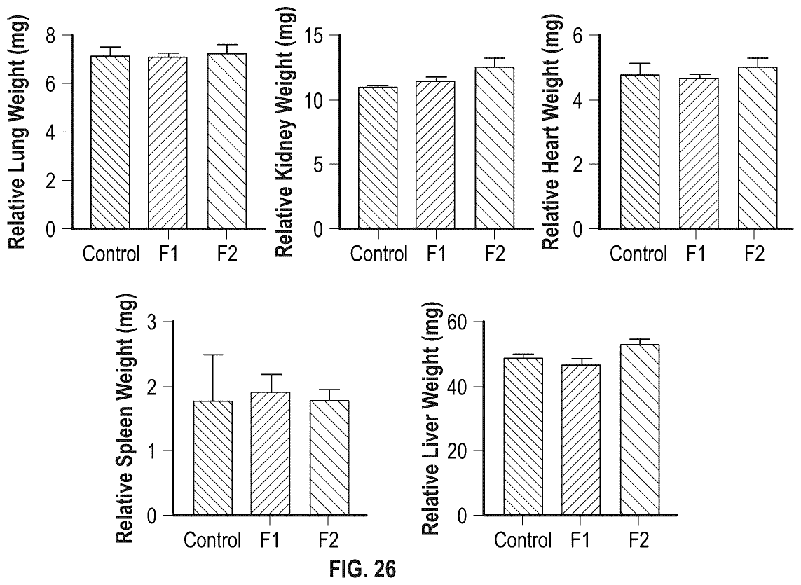

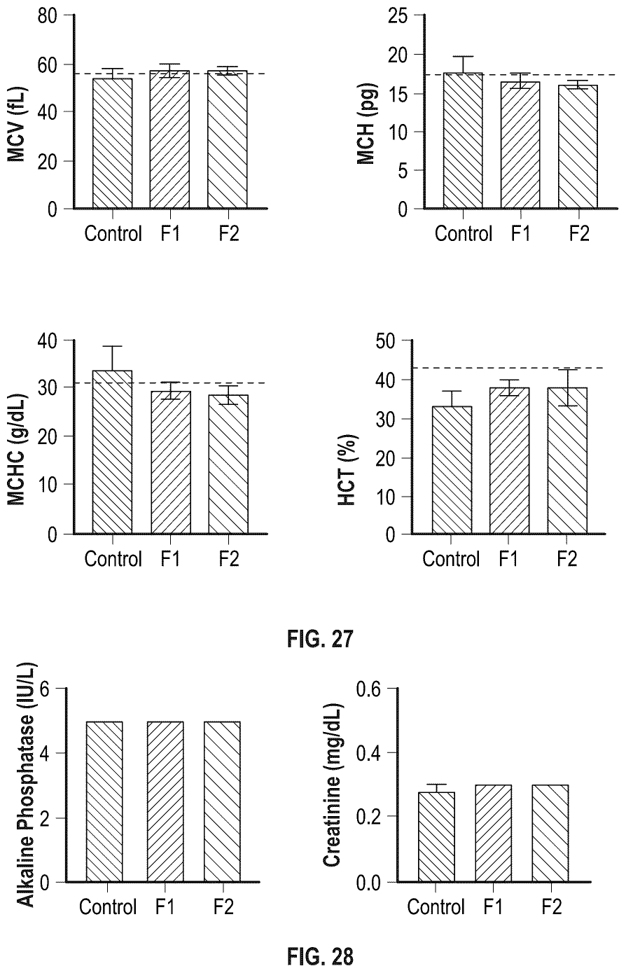

Consistent with the aforementioned results, no aberrant change in mice weights was observed among all groups during the study (FIG. 25). The safety profile of PTX loaded F2 NPs was also determined by CBC analysis, blood chemistry, and organ histology. any significant change in the organ weight of the F1 and F2 NPs treated group was not observed compared to the control group (FIG. 26). The CBC and blood chemistry analysis corroborated that no toxicity is associated with positively charged F2 NPs (FIG. 8C, FIG. 8D, FIG. 27, and FIG. 28). Similarly, no aberrant histological changes were observed in the liver, kidney, and spleen of the group treated with paclitaxel containing F2 NPs in comparison to the control group (FIG. 24).

### Discussion

Here, novel poly-L-histidine based PLGA nanoformulations were established for delivering small molecules and nucleic acid-based analogs with minimal toxicity. Several cationic polymer based nanoformulations have gained interest due to their increased ionic interaction with the plasma membrane and improved intracellular trafficking owing to endosmolytic properties (Samal et al. Chem. Soc. Rev. 2012, 41 (21), 7147-94). In particular, poly-β-amino ester (PBAE), and polyethyleneimine (PEI), based formulations have been explored to improve the nanoparticles' cytoplasmic delivery in prior studies (Wahane et al. Molecules 2020, 25 (12), 2866).

Though it shows promise to some extent, the toxicity associated with high positive surface charge density results in cell-based cytotoxicity, nonspecific binding to serum proteins, and off-target tissue accumulation circumvent the clinical translation of such polymeric formulations (Lv et al. J. Control. Release 2006, 114 (1), 100-09). Various counter ions or polymers have been used to minimize the cationic surface charge density to overcome the challenges mentioned above. In particular, several studies reported that PBAE:PLGA mixture at a ratio of 5:95 (% w/w) renders less toxicity as compared to PBAE:PLGA at 15:85 (% w/w) for numerous biomedical applications (Fields et al. J. Control. Release 2012, 164 (1), 41-48; Fields et al. Adv. Healthc. Mater. 2015, 4 (3), 361-66). Though these blends of polymers have attained some success, still the production of these polymers requires optimization of synthetic protocols and additional quality control-based assessments. Hence, novel biocompatible nanoformulations that rely on less synthetic protocols without compromising their functional activity need to be explored.

On this front, histidine-based formulations have generated ample interest. Prior studies centered on synthesis of new PLGA or PLGA-PEG-based histidine polymers for effective drug delivery (Hong et al. Acta Biomater. 2014, 10 (3), 1259-71; Lee et al. J. Control. Release 2003, 90 (3), 363-74). In addition, poly-L-histidine-co-block polymers were used for poly-L-histidine-PEG pH-sensitive polymeric micelles to deliver chemotherapeutic drugs (Lee et al. J. Control. Release 2003, 90 (3), 363-74). Though promising to some extent, still mechanistic understanding of cellular uptake of these polymers need to be explored. Moreover, aforementioned methods involve chemical conjugation of histidine or poly-L-histidine units with polymers which needs elaborative synthesis and quality control analysis. Hence, novel PLGA:poly-L-histidine (F2) based formulations that are easy to scale up without affecting their transfection efficiency were tested and optimized. First, the comprehensive physico-biochemical attributes of PLGA NPs containing either monomeric or polymeric histidine moieties formulated using double emulsion solvent evaporation method were studied. Herein, it was demonstrated that the PLGA:poly-L-histidine formulation shows positive surface charge density, and narrower size distribution properties as compared to conventional PLGA (F1) formulations. Further, it was also established that substituting DCM with acetone:DCM as solvent decreases the size of NPs with lower PDI. This could be due to the solvent displacement effect as acetone is an amphiphilic organic solvent which diffuses out into the aqueous phase leaving behind PLGA polymer in smaller DCM oil globules (Beck-Broichsitter et al. Eur. J. Pharm. Sci. 2010, 41 (2), 244-53). Further, evaporation of DCM solvent allows precipitation of PLGA polymer into NPs. It was further established that C6 fluorophore containing NP formulations with PLGA:poly-L-histidine at a ratio of 4.9:0.1 possess optimal cationic surface charge density and retains the superior transfection efficiency as compared to other tested formulations. The optimized PLGA:poly-L-histidine formulation is easy to produce compared to other cationic synthetic polymers for efficient gene delivery.

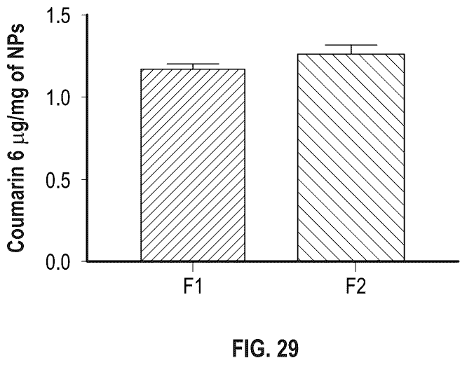

Though F2 formulation showed similar loading of C6 compared to the standard F1 PLGA formulation (FIG. 29), the F2 formulation possesses a higher fluorescent signal in confocal microscopy, confirming its promising transfection efficiency in comparison to the standard F1 PLGA formulation. The contrast-matched SANS results confirmed that F2 formulations contain patches populated over the PLGA NP surface that are likely composed of poly-L-histidine domains giving rise to the positive charge. Without being bound by theory, it is proposed that these domains might be locally packed yielding significant structure factor at high q. Further, PLGA NPs have been reported to have moderate cellular uptake properties, and their mechanism of uptake is known to be endocytosis (Cartiera et al. Biomaterials 2009, 30 (14), 2790-98). Several histidine-based co-block polymers have been reported, but their cellular transport mechanism is not well understood (He et al. Pharmaceutics 2020, 12 (8), 1-31).

Here, the cellular uptake mechanism of PLGA:poly-L-histidine (F2) NPs was also investigated. It was demonstrated that F2 formulation undergoes cellular uptake via clathrin-dependent pathway by inclusive endocytosis studies supported by confocal and flow-cytometry. It was also established that F2 NPs can encapsulate nucleic acid-based analogs and small molecule-based drug candidates. For nucleic acid analogs, PNA-155 targeting oncomiR-155 that is overexpressed in numerous solid tumors and diffuse tumors like lymphomas and leukemia was used. Prior studies reported that conventional PLGA NPs could encapsulate antisense and anti-miR PNAs without affecting their properties (Malik et al. J. Control. Release 2020, 327 (March), 406-19; Malik et al. Methods X 2020, 7, 101115). Consistent with earlier observations, F2 formulations were observed to encapsulate PNA-155 without affecting its integrity.

Furthermore, histidine was shown not to affect the release properties of PNA from F2 formulations. The F2 NPs showed significant knockdown of target oncomiR-155 in vitro. The systemic administration of F2 NPs in xenograft mice resulted in optimal tumor targeting. Next, F2 formulations containing PNA-155 showed significant tumor growth inhibition in xenograft mice than F1 formulation containing PNA-155 after systemic administration. The functional activity was also established by gene expression analysis of miR-155 and its downstream target genes. Similarly, immunohistochemistry analysis confirmed reduced proliferation of F2 NPs treated tumors. To broaden the application of the disclosed formulations, encapsulated PTX was encapsulated in F2 formulation, and it was compared with PTX F1 formulation. In vitro results indicated that PLGA:poly-L-histidine NPs were able to load a substantial amount of PTX and show optimal efficacy in cell proliferation and apoptotic assays. In vivo evaluations also resulted in significantly reduced tumor growth after treatment with F2 NPs containing PTX than F1 NPs. Based on extensive toxicity evaluation, both the PNA-155 and PTX loaded F2 NPs were found to be safe in vivo, establishing F2 formulation as a clinically viable approach. Nanoformulations, including FDA-approved Doxil, target the tumor site by EPR effect (Dawidczyk et al. Nanomedicine Nanotechnology, Biol. Med. 2017). Herein, it was demonstrated that F2 formulation containing synthetic nucleic acid analogs or small molecule-based drug candidates possess moderate surface charge density that can target the tumor by EPR and inhibit its growth after systemic administration. In the future, it would be noteworthy to study the ligand coating of PLGA:poly-L-histidine NPs to further achieve active targeting and henceforth broaden the application to deliver a range of drug candidates.

### Conclusions

Overall, a novel PLGA-histidine based platform was developed that can deliver small molecules followed by gene delivery for a plethora of therapeutic applications. These formulations exhibit promising features in terms of better efficacy, minimal toxicity and are easy to produce for gene delivery-based applications. Hence, polymeric PLGA:poly-L-histidine formulations were developed and optimized, considering scalable aspects of manufacturing, effectiveness, toxicity, and established their feasibility for successful clinical translation.

### Experimental Methods

### Materials

Coumarin-6, nile red, L-histidine, poly-L-histidine, polyvinyl alcohol (PVA), dichloromethane (DCM), acetone, trehalose, and paclitaxel were purchased from Sigma Aldrich. Lipofectamine RNAiMAX was purchased from Thermo Fisher Scientific. PLGA (50:50, lactic acid:glycolic acid, ester-terminated, 0.26-0.54 g/dL) polymer was purchased from Lactel Absorbable Polymers (Durect Corporation, USA). PLGA-L-histidine polymer was purchased from PolySciTech, Akina, Inc (USA). Boc-protected PNA monomers used to synthesize PNA were procured from ASM, Germany. TAMRA (5-carboxytetramethylrhodamine) dye was procured from VWR (Pennsylvania, USA). Deuterium oxide (D₂O) was obtained from Cambridge Isotope Laboratories (USA).

### Nanoparticles Formulation

40 mg of PLGA polymer was soaked for 4-5 h in DCM (500 μL). The aqueous encapsulant (˜40 μL) was added to the polymer solution while being vortexed, followed by ultra-sonication (3×10 s) using a probe sonicator (SONICS Vibracell, CT, USA) to obtain the first water in oil (w/o) emulsion. This w/o emulsion was then added dropwise to 1 mL of 5% PVA while being vortexed, followed by ultra-sonication (3×10 s) forming a w/o/w double emulsion. Further, double emulsion was added dropwise to 0.3% PVA solution stirring at 700 RPM at room temperature and kept overnight to allow the DCM to evaporate. NPs were then centrifuged at (9500 RPM for 10 mins) and washed (3×) with ice-cold water. After washing, the nanoparticle pellet was resuspended with trehalose aqueous solution (5 mg/mL) and lyophilized overnight. After lyophilization NPs were stored at −20° C. NPs were also formulated using 500 μL of acetone:DCM (2:1) as the organic phase and were washed twice at 15,000 RPM for 25 mins. For the formulation of coumarin-6 or nile red loaded NPs, 50 μL of 1 mg/mL stocks of coumarin-6 and nile red in DCM was added to 450 μL of organic phase (DCM or acetone:DCM) followed by the L water addition to make the first w/o emulsion. Paclitaxel stock was prepared in DCM (1 mg/mL) and 50 μL was added to the acetone:DCM (2:1) organic phase followed by 40 L of water addition to formulate the NPs. PNA-155 loaded NPs were formulated by adding 40 μL of 1 mM aqueous PNA stock to 500 μL of the organic phase (acetone:DCM, 2:1). PLGA:poly-L-histidine NPs were formulated using different weight ratios of PLGA and poly-L-histidine ranging from 3:2 to 4.9:0.1. PLGA-L-histidine NPs were prepared using PLGA-L-histidine polymer from PolySciTech, Akina, Inc (USA) containing 13.5% L-histidine. PLGA+L-histidine NPs were prepared by using a ratio of 1:6.3 of L-histidine and PLGA to achieve 13.5% of L-histidine amount.

### SANS Sample Preparation

For SANS analysis, blank nanoparticle formulations were formulated using the already described double emulsion solvent evaporation method. Each NPs formulation analyzed by SANS contained 0.75% of histidine content in 47% D₂O solvent.

### Size and Zeta Potential Analysis of Nanoparticles

NPs were characterized using a Zetasizer Nano ZS (Malvern Panalytical Inc., Westborough, MA, USA). Non-invasive back scatter technology was used to measure particle size and poly-dispersity index by dynamic light scattering at 25° C. and refractive index of 1.33. The laser doppler micro-electrophoresis technique was used to measure the zeta potential at 25° C. Different batches (three replicates) were analyzed for each group and average values were reported.

### PNA Synthesis

PNA was synthesized using solid-phase synthesis with 4-Methylbenzhydrylamine (also called MBHA) resin as solid support and Boc-protected monomers. Further, TAMRA dye was conjugated to the N-termini with mini-PEG-3 as a linker. The following PNA sequence was synthesized:

| | PNA-155: |
|---|---|
| (SEQ ID NO: 15) | |
| | TAMRA-OOO-RRR-ACCCCTATCACGATTAGCATTAA-R. |

A cleavage cocktail comprising of trifluoroacetic acid:trifluoromethanesulfonic acid:m-cresol:thioanisole (6:2:1:1) was used to cleave the PNA. Diethyl ether was used to precipitate the cleaved PNA followed by its HPLC purification. The molecular weight of PNA was detected using MALDI-TOF. The molar extinction coefficient of the PNA was measured using the sum of extinction coefficient of each monomer. Molar extinction coefficient was also used to determine the concentration of the PNA via UV-Vis spectroscopy.

### Cell Culture

HeLa (CCL-2™), PBMC (PCS-800-011™), and HEK293 (CRL-1573™) cells were purchased from ATCC (Virginia, USA). U2932 cells were procured from Leibniz Institute (DSMZ, Germany). HeLa and HEK293 cells were seeded in Petri dishes (10 cm) using eagle's minimum essential medium (EMEM) (ATCC® 30-2003™) supplemented with 10% fetal bovine serum (FBS) (Gibco®) and 1% PenStrep. Cells were passaged at 80% confluency. PBMC and U2932 cells were cultured in 75 mm³ flask using RPMI-1640 (ATCC® 30-2001™) media supplemented with 10% FBS and 1% PenStrep.

### Flow Cytometry Analysis

HeLa cells (100,000) were seeded in a 12 well plate with EMEM (ATCC® 30-2003™) media supplemented with 10% FBS (Gibco®) overnight and treated with 2 mg/mL NPs dose. After 24 h, cells were washed with PBS (4×) to remove the non-internalized NPs and trypsinized (0.25% trypsin-EDTA (Gibco®) at 37° C. for 5 mins). 1 mL of media was then added and cells were centrifuged at 2000 RPM for 3 mins at 4° C. The cell pellets were then resuspended in 300 μL of 4% paraformaldehyde. For uptake in U2932 cells, 200,000 cells were seeded in 12 well plate with RPMI (ATCC® 30-2001™) media and treated with 1 mg/mL NPs. The next day, the cells were fixed and processed for flow cytometry analysis. Flow cytometry was performed using LSR Fortessa X-20 Cell Analyzer (BD Biosciences, CA) and FlowJo analysis software was used to analyze the results.

### Confocal Microscopy

50,000 HeLa cells were placed on coverslips in a 24 well plate overnight and treated with 500 μL of 2 mg/mL NPs suspension. After 24 h, cells were washed with PBS (4×) to remove non-internalized NPs and fixed by incubating the cells in 4% paraformaldehyde (PFA) for 10 mins at room temperature (rt). Further, the cells were permeabilized using 0.1% Triton-X (Thermo Fisher Scientific) at rt for 10 mins. Followed by washing, the cell culture coverslips were mounted on glass slides containing a drop of ProLong™ Diamond Antifade Mountant with DAPI (Life Technologies, Carlsbad, CA, USA). The glass slides containing coverslips were then kept at 4° C. overnight and samples were imaged by confocal microscope (Nikon A1R spectral).

For time-dependent cellular uptake, 50,000 HeLa cells per well were treated with NPs (2 mg/mL) for a duration of 2, 4, 6, 12, and 24 h. To evaluate cellular uptake in the presence of endocytosis inhibitors, 50,000 HeLa cells were pretreated with Amiloride (1 mM), Chlorpromazine (10 μg/mL), and Genistein (200 μM) at 37° C. for 30 mins. Cells were washed with PBS (2×) and treated for 2 h with 2 mg/mL NPs followed by processing for imaging. For temperature-dependent cellular uptake study, 50,000 HeLa cells per well were preconditioned at 37° C. and 4° C. for 30 mins. The cells were then treated with a 2 mg/mL NPs dose for 2 h and processed for imaging.

### Loading Study

The PNA-155 loaded lyophilized F1 & F2 NPs were resuspended in 200 μL DCM and were allowed to shake at 1000 RPM at 37° C. for 8 h to dissolve the PLGA polymeric core. Further, the same volume of sodium acetate buffer (pH 5.8) was added to the NPs and kept at 1000 RPM at 37° C. for another 4 h to extract the PNA in the aqueous phase. The NPs were then centrifuged at 15000 RPM for 10 mins and a sample was drawn from the supernatant aqueous phase. The concentration of PNA in the supernatant was measured using Nanodrop One (Thermo Scientific, MA). To determine the loading of Paclitaxel, F1 & F2 NPs were dispersed in 200 μL DCM and allowed to shake for 8 h. The NPs were then centrifuged at 15000 RPM for 10 mins. 100 μL of DCM was taken out in a separate tube and DCM was allowed to evaporate for 3 h in the chemical hood. After that 50 μL of methanol was added to precipitate the PLGA polymer and was centrifuged at 15000 RPM for 10 mins. The supernatant (˜10 μL) was taken for measuring absorbance at 228 nm using Nanodrop One. The amount of paclitaxel loaded in NPs was calculated using a standard curve of paclitaxel at 228 nm. To evaluate loading of coumarin-6 NPs, 200 μL DCM was added to the lyophilized NPs and allowed to shake for 8 h. 100 μL of DCM was taken out in a separate tube and 200 μL of methanol was added to precipitate the PLGA polymer followed by centrifugation at 15000 RPM to pellet the precipitated PLGA polymer. 100 μL of the supernatant was then taken for measuring fluorescence. The amount of coumarin 6 was calculated using their standard curves at excitation/emission wavelengths of 457/505 nm.

### Release Study

NPs were resuspended in 300 μL of PBS (Gibco®) and were allowed to shake at 300 RPM at 37° C. The samples were collected at different time points by centrifugation of NPs at 15,000 RPM for 10 mins. NPs were then resuspended in fresh PBS after each time point. The amount of PNA released at each time point was determined by calculating the absorbance at 260 nm using Nanodrop One.

### Scanning Electron Microscopy (SEM) and Transmission Electron Microscopy (TEM) of NPs

NPs mounted on carbon black tape were sputter-coated for 2 mins. The images were taken at 10,000× and 50,000× magnifications at the voltage of 2.0 kV on a FEI Nova NanoSEM 450. ImageJ software (NIH, Bethesda, MD) was used to measure the particle size distribution. For TEM imaging, NPs were resuspended in water and were stained with 1% uranyl acetate on carbon grids with 400 mesh copper (CF400-CU) for 5 mins. Imaging was performed using FEI Tecnai at 80 kV voltage.

### Gene Expression (RT-PCR)

200,000 U2932 cells were seeded overnight and pre-treated with 1 mg/mL NPs dose. The cells were then centrifuged, and RNA was separated using RNeasy Mini Kit (Qiagen, Germany). miR-155 levels were measured using TaqMan™ MicroRNA Assay (Assay ID: 467534_mat). miR-155 reverse transcriptase (RT) primers, 10× RT buffer, 100 mM dNTPs with RNase inhibitor (Applied Biosystem, CA) were used to synthesize cDNA at the temperature conditions of 16° C. for 30 mins, 42° C. for 30 mins, and 85° C. for 5 mins in a thermal cycler (T100™, Bio-Rad, CA). Primers for miR-155, FOXO3A, BACH1, and TaqMan™ Universal Master Mix II, with UNG (Applied Biosystem, CA) were employed to amplify the cDNA at the temperature conditions of 50° C. for 2 mins, 95° C. for 10 mins, 95° C. for 15 s and 60° C. for 60 s, for 40 cycles. TaqMan U6 snRNA (Assay ID: 001973) and GAPDH (Assay ID: Hs02786624_g1) was used as the endogenous control for miR-155 and mRNA quantification respectively. Results were normalized relative to the control samples.

### Endotoxin Assay

The endotoxin levels in nanoformulations were tested using Pierce™ LAL Chromogenic Endotoxin Quantitation Kit (Thermo Fisher Scientific). Nanoparticles were resuspended at a concentration of 4 mg/mL in endotoxin free water (Cytiva, Fisher Scientific) and diluted 10-fold to a concentration of 400 μg/mL. pH of the samples was adjusted around 6-8 followed by addition of the reagents and measuring absorbance at 405 nm. Endotoxin levels were calculated using a standard curve as per the reference endotoxin standard provided by the manufacturer.

### Trypan Blue Assay

200,000 PBMC cells were seeded in a plate (12 well) overnight and treated with 2 mg/mL blank F2 NPs and 150 μL of lipofectamine. After 24 h, cells were stained with trypan blue dye and total live cells were determined by cell counter (Bio-Rad, USA). For cytotoxicity assay in HeLa cells, 50,000 HeLa cells were seeded in plates (24 well) and treated with paclitaxel (PTX) F2 NPs for 24 h, and cell viability was measured by staining with trypan blue dye.

### Cytotoxicity (MTT) Assay

2,500 HEK293 cells per well were seeded in a 96 well plate and incubated with blank NPs at 2 mg/mL dose and 15 μL of lipofectamine for 72 hours. Further, cells were washed with PBS (2×) and cultured in fresh media with 20 μL of MTS reagent (CellTiter, Promega) at 37° C. After 1 hour, absorbance at 490 nm was measured using iMark plate reader (Bio-Rad) and viability of the cells was calculated using fold change in optical density of treated group relative to the control.

### Annexin V Apoptosis Assay

200,000 HeLa cells were seeded in a 12 well plate and further treated with 2 mg/mL blank F2 NPs and 3 μL lipofectamine. For paclitaxel treatments, PTX F2 NPs at 2 mg/mL were tested against PTX suspension at an equivalent dose in 200,000 HeLa cells. After 24 h, cells were washed twice with PBS and trypsinized for 5 mins at 37° C. The number of cells undergoing apoptosis was determined by PE Annexin V Apoptosis Detection Kit I (BD, Franklin Lakes, NJ, USA). The cells were centrifuged at 2000 RPM for 3 mins at 4° C., resuspended in binding buffer, and cell count was determined using a cell counter. 1×10′ cells (100 μL) were then stained by incubation with 10 μL Annexin PE and 10 μL 7AAD for 15 mins at RT (25° C.) in the dark. Control samples containing unstained cells, cells stained with Annexin PE, and cells stained with 7AAD were also prepared for compensation setup for flow cytometry analysis. After 15 mins of incubation, 400 μL of Annexin V binding buffer was added to the cells and analyzed by flow cytometry.

### In Vivo Studies in Xenograft Mice

In vivo studies were performed in 5-6 weeks old female NSG (NOD.Cg-Prkdc^{scid} Il2rg^{t,1wjl}/SzJ, strain 005557) mice procured from Jackson Labs. The animals were housed at the UConn animal facility as per IACUC guidelines and protocols. U2932 tumors were grown on the right flank of 5-6 weeks old mice by injecting 1×10⁷ U2932 cells subcutaneously. Once the tumors reached 100-200 mm³ volume, the mice were divided into five treatment groups (n≥5) and injected with PBS, PNA-155 F1, PNA-155 F2, PTX F1, and PTX F2 NPs. The NPs were dispersed in appropriate volume of PBS and sonicated thoroughly. For PNA-155 treatment group, three doses of 3 mg NPs were injected retroorbitally over 7 days. For PTX treatment group, three doses of 2 mg NPs were administered via tail vein over 7 days. The tumor volume of mice was calculated every day by vernier calipers. Once the tumor volume reached 2000 mm³, mice were euthanized. Complete blood count (CBC) analysis was conducted on the whole blood collected from the mice using Sysmex CBC analyzer. The blood samples were then centrifuged (4500 RPM for 10 mins) to isolate the plasma. Further, the plasma samples were submitted to Antech diagnostics (Irvine, CA) for blood chemistry analysis including creatinine, alanine aminotransferase (ALT), alkaline phosphatase, lactate dehydrogenase (LDH), aspartate aminotransferase (AST) and blood urea nitrogen (BUN). Tumor from the PNA-155 treated and control group were dissociated using dispase (STEM cell Technologies Inc., WA) and collagenase type I (Worthington Biochemical Corp., NJ) to prepare a single-cell suspension. The cells were further treated with RBC lysis buffer followed by removal of mouse cells by a mouse cell depletion kit (Miltenyi Biotech, CA). The enriched U2932 cells were processed further for gene expression analysis to measure the miR-155 levels and its downstream targets, i.e., BACH1 and FOXO3A. The tumor and vital organs (liver, kidney, spleen, lungs, heart) were carefully isolated, weighed, and fixed in the 10% NBF solution. The sections (5 μm) of formalin-fixed paraffin-embedded liver and kidney were stained by hematoxylin and eosin for the histological analysis. The sections (5 μm) of the formalin-fixed paraffin-embedded tumor were heated (95° C., 20 min) in citrate buffer (10 mM) for antigen retrieval, followed by incubation with primary antibodies. The working concentrations of rabbit anti-Ki67 (Cell Signaling Technology, USA) antibody was 1:100. The antigen-primary antibody complexes were probed with alexfluor-647 tagged secondary antibody and the images were captured using a Zeiss Inverted Confocal microscope (Model 510).

### Biodistribution Study

For biodistribution studies, five U2932 xenograft mice with 100-200 mm³ tumors were randomly chosen and injected with PBS (n=1) and 3 mg nile red containing PLGA: poly-L-histidine (F2) NPs (n=4). Animals were euthanized after 4 hours (n=2) and 8 hours (n=2) of systemic administration of NPs and their tumor as well as other organs were harvested. The harvested tumors were processed for cryosectioning followed by staining of the nucleus. The NPs accumulation in the tumor was studied by confocal microscopy.

### REFERENCES

(1) Smith, S. A.; Selby, L. I.; Johnston, A. P. R.; Such, G. K. The Endosomal Escape of Nanoparticles: Toward More Efficient Cellular Delivery. Bioconjug. Chem. 2019, 30 (2), 263-272.
(2) Cervia, L. D.; Chang, C. C.; Wang, L.; Yuan, F. Distinct Effects of Endosomal Escape and Inhibition of Endosomal Trafficking on Gene Delivery via Electrotransfection. PLoS One 2017, 12 (2), 1-18.
(3) Boukany, P. E.; Wu, Y.; Zhao, X.; Kwak, K. J.; Glazer, P. J.; Leong, K.; Lee, L. J. Nonendocytic Delivery of Lipoplex Nanoparticles into Living Cells Using Nanochannel Electroporation. Adv. Healthc. Mater. 2014, 3 (5), 682-689.
(4) Martinet, W.; Schrijvers, D. M.; Kockx, M. M. Nucleofection as an Efficient Nonviral Transfection Method for Human Monocytic Cells. Biotechnol. Lett. 2003, 25 (13), 1025-1029.
(5) Zeitelhofer, M.; Vessey, J. P.; Xie, Y.; Tubing, F.; Thomas, S.; Dahm, R. High-Efficiency Transfection of Mammalian Neurons via Nucleofection. Nat. Protoc. 2007, 2 (7), 1692-1704.
(6) Cardarelli, F.; Digiacomo, L.; Marchini, C.; Amici, A.; Salomone, F.; Fiume, G.; Rossetta, A.; Gratton, E.; Pozzi, D.; Caracciolo, G. The Intracellular Trafficking Mechanism of Lipofectamine-Based Transfection Reagents and Its Implication for Gene Delivery. Sci. Rep. 2016, 6 (May), 1-8.
(7) Erbacher, P.; Roche, A. C.; Monsigny, M.; Midoux, P. Putative Role of Chloroquine in Gene Transfer into a Human Hepatoma Cell Line by DNA/Lactosylated Polylysine Complexes. Exp. Cell Res. 1996, 225 (1), 186-194.
(8) Mashal, M.; Attia, N.; Martínez-Navarrete, G.; Soto-Sánchez, C.; Fernández, E.; Grijalvo, S.; Eritja, R.; Puras, G.; Pedraz, J. L. Gene Delivery to the Rat Retina by Non-Viral Vectors Based on Chloroquine-Containing Cationic Niosomes. J. Control. Release 2019, 304 (February), 181-190.
(9) Zaitsev, S.; Buchwalow, I.; Haberland, A.; Tkachuk, S.; Zaitseva, I.; Haller, H.; Böttger, M. Histone H1-Mediated Transfection: Role of Calcium in the Cellular Uptake and Intracellular Fate of H1-DNA Complexes. Acta Histochem. 2002, 104 (1), 85-92.
(10) Kulkarni, V. I.; Shenoy, V. S.; Dodiya, S. S.; Rajyaguru, T. H.; Murthy, R. R. Role of Calcium in Gene Delivery. Expert Opin. Drug Deliv. 2006, 3 (2), 235-245.
(11) Brock, D. J.; Kondow-Mcconaghy, H. M.; Hager, E. C.; Pellois, J. P. Endosomal Escape and Cytosolic Penetration of Macromolecules Mediated by Synthetic Delivery Agents. Bioconjug. Chem. 2019, 30 (2), 293-304.
(12) Pei, D.; Buyanova, M. Overcoming Endosomal Entrapment in Drug Delivery. Bioconjug. Chem. 2019, 30 (2), 273-283.
(13) Zhou, J.; Patel, T. R.; Fu, M.; Bertram, J. P.; Saltzman, W. M. Octa-Functional PLGA Nanoparticles for Targeted and Efficient SiRNA Delivery to Tumors. Biomaterials 2012, 33 (2), 583-591.
(14) Hasan, W.; Chu, K.; Gullapalli, A.; Dunn, S. S.; Enlow, E. M.; Luft, J. C.; Tian, S.; Napier, M. E.; Pohlhaus, P. D.; Rolland, J. P.; Desimone, J. M. Delivery of Multiple SiRNAs Using Lipid-Coated PLGA Nanoparticles for Treatment of Prostate Cancer. Nano Lett. 2012, 12 (1), 287-292.
(15) Wang, D.; Robinson, D. R.; Kwon, G. S.; Samuel, J. Encapsulation of Plasmid DNA in Biodegradable Poly(D,L-Lactic-Co-Glycolic Acid) Microspheres as a Novel Approach for Immunogene Delivery. J. Control. Release 1999, 57 (1), 9-18.
(16) Ravi Kumar, M. N. V.; Bakowsky, U.; Lehr, C. M. Preparation and Characterization of Cationic PLGA Nanospheres as DNA Carriers. Biomaterials 2004, 25 (10), 1771-1777.
(17) Cheng, C. J.; Bahal, R.; Babar, I. A.; Pincus, Z.; Barrera, F.; Liu, C.; Svoronos, A.; Braddock, D. T.; Glazer, P. M.; Engelman, D. M.; Saltzman, W. M.; Slack, F. J. MicroRNA Silencing for Cancer Therapy Targeted to the Tumour Microenvironment. Nature 2015, 518 (7537), 107-110.
(18) Bahal, R.; Mcneer, N. A.; Quijano, E.; Liu, Y.; Sulkowski, P.; Turchick, A.; Lu, Y.-C.; Bhunia, D. C.; Manna, A.; Greiner, D. L.; Brehm, M. A.; Cheng, C. J.; López-Giráldez, F.; Ricciardi, A.; Beloor, J.; Krause, D. S.; Kumar, P.; Gallagher, P. G.; Braddock, D. T.; Saltzman, W. M.; Ly, D. H.; Glazer, P. M. In vivo Correction of Anaemia in β-Thalassemic Mice by ΓPNA-Mediated Gene Editing with Nanoparticle Delivery. Nat. Commun. 2016, 7.
(19) Gupta, A.; Bahal, R.; Gupta, M.; Glazer, P. M.; Saltzman, W. M. Nanotechnology for Delivery of Peptide Nucleic Acids (PNAs). J. Control. Release 2016.
(20) McNeer, N. A.; Anandalingam, K.; Fields, R. J.; Caputo, C.; Kopic, S.; Gupta, A.; Quijano, E.; Polikoff, L.; Kong, Y.; Bahal, R.; Geibel, J. P.; Glazer, P. M.; Mark Saltzman, W.; Egan, M. E. Nanoparticles That Deliver Triplex-Forming Peptide Nucleic Acid Molecules Correct F508del CFTR in Airway Epithelium. Nat. Commun. 2015, 6.
(21) Danhier, F.; Ansorena, E.; Silva, J. M.; Coco, R.; Le Breton, A.; Préat, V. PLGA-Based Nanoparticles: An Overview of Biomedical Applications. J. Control. Release 2012, 161 (2), 505-522.
(22) Cartiera, M. S.; Johnson, K. M.; Rajendran, V.; Caplan, M. J.; Saltzman, W. M. The Uptake and Intracellular Fate of PLGA Nanoparticles in Epithelial Cells. Biomaterials 2009, 30 (14), 2790-2798.
(23) Peetla, C.; Jin, S.; Weimer, J.; Elegbede, A.; Labhasetwar, V. Biomechanics and Thermodynamics of Nanoparticle Interactions with Plasma and Endosomal Membrane Lipids in Cellular Uptake and Endosomal Escape. Langmuir 2014, 30 (25), 7522-7532.
(24) M. Cardoso, M.; N. Peca, I.; C. A. Roque, A. Antibody-Conjugated Nanoparticles for Therapeutic Applications. Curr. Med. Chem. 2012.
(25) Gwak, S. J.; Macks, C.; Bae, S.; Cecil, N.; Lee, J. S. Physicochemical Stability and Transfection Efficiency of Cationic Amphiphilic Copolymer/PDNA Polyplexes for Spinal Cord Injury Repair. Sci. Rep. 2017, 7 (1), 1-10.
(26) JR, H.; KT, K.; SJ, G.; R, P.; MB, J.; MR, C.; L, R.; M, V.; I, M.; J S, L.; KA, A. A Cationic Amphiphilic Co-Polymer as a Carrier of Nucleic Acid Nanoparticles (Nanps) for Controlled Gene Silencing, Immunostimulation, and Biodistribution. Nanomedicine 2020, 23.
(27) SJ, G.; J, N.; J, Z.; B, G.; C, M.; S, B.; K, W.; J S, L. Cationic, Amphiphilic Copolymer Micelles as Nucleic Acid Carriers for Enhanced Transfection in Rat Spinal Cord. Acta Biomater. 2016, 35, 98-108.
(28) Liu, G.; Molas, M.; Grossmann, G. A.; Pasumarthy, M.; Perales, J. C.; Cooper, M. J.; Hanson, R. W. Biological Properties of Poly-L-Lysine-DNA Complexes Generated by Cooperative Binding of the Polycation. J. Biol. Chem. 2001, 276 (37), 34379-34387.
(29) Hong, C. A.; Son, H. Y.; Nam, Y. S. Layer-by-Layer SiRNA/Poly(L-Lysine) Multilayers on Polydopamine-Coated Surface for Efficient Cell Adhesion and Gene Silencing. Sci. Rep. 2018, 8 (1), 1-7.
(30) Hu, J.; Lou, Y.; Wu, F. Improved Intracellular Delivery of Polyarginine Peptides with Cargoes. J. Phys. Chem. B 2019, 123 (12), 2636-2644.
(31) Lozano, M. V.; Lollo, G.; Alonso-Nocelo, M.; Brea, J.; Vidal, A.; Torres, D.; Alonso, M. J. Polyarginine Nanocapsules: A New Platform for Intracellular Drug Delivery. J. Nanoparticle Res. 2013, 15 (3).
(32) Bilalis, P.; Tziveleka, L. A.; Varlas, S.; Iatrou, H. PH-Sensitive Nanogates Based on Poly(l-Histidine) for Controlled Drug Release from Mesoporous Silica Nanoparticles. Polym. Chem. 2016, 7 (7), 1475-1485.
(33) Sun, M.; Zhu, Z.; Wang, H.; Han, C.; Liu, D.; Tian, L.; Yang, X.; Pan, W. Surface Density of Polyarginine Influence the Size, Zeta Potential, Cellular Uptake and Tissue Distribution of the Nanostructured Lipid Carrier. Drug Deliv. 2017, 24 (1), 519-526.
(34) Wen, Y.; Guo, Z.; Du, Z.; Fang, R.; Wu, H.; Zeng, X.; Wang, C.; Feng, M.; Pan, S. Serum Tolerance and Endosomal Escape Capacity of Histidine-Modified PDNA-Loaded Complexes Based on Polyamidoamine Dendrimer Derivatives. Biomaterials 2012, 33 (32), 8111-8121.
(35) Pack, D. W.; Putnam, D.; Langer, R. Design of Imidazole-Containing Endosomolytic Biopolymers for Gene Delivery. Biotechnol. Bioeng. 2000.
(36) Midoux, P.; Monsigny, M. Efficient Gene Transfer by Histidylated Polylysine/PDNA Complexes. Bioconjug. Chem. 1999, 10 (3), 406-411.
(37) Hong, W.; Chen, D.; Jia, L.; Gu, J.; Hu, H.; Zhao, X.; Qiao, M. Thermo- and PH-Responsive Copolymers Based on PLGA-PEG-PLGA and Poly(l-Histidine): Synthesis and in vitro Characterization of Copolymer Micelles. Acta Biomater. 2014, 10 (3), 1259-1271.
(38) Liu, R.; L1, D.; He, B.; Xu, X.; Sheng, M.; Lai, Y.; Wang, G.; Gu, Z. Anti-Tumor Drug Delivery of PH-Sensitive Poly(Ethylene Glycol)-Poly(L-Histidine-)-Poly(L-Lactide) Nanoparticles. J. Control. Release 2011, 152 (1), 49-56.
(39) Moro, J.; Tomé, D.; Schmidely, P.; Demersay, T. C.; Azzout-Marniche, D. Histidine: A Systematic Review on Metabolism and Physiological Effects in Human and Different Animal Species. Nutrients 2020.
(40) Chen, Q. R.; Zhang, L.; Luther, P. W.; Mixson, A. J. Optimal Transfection with the HK Polymer Depends on Its Degree of Branching and the PH of Endocytic Vesicles. Nucleic Acids Res. 2002, 30 (6), 1338-1345.
(41) Little, S. R.; Lynn, D. M.; Puram, S. V; Langer, R. Formulation and Characterization of Poly (h Amino Ester) Microparticles for Genetic Vaccine Delivery. J Control release 2005, 107, 449-462.
(42) Omidi, Y.; Kafil, V. Cytotoxic Impacts of Linear and Branched Polyethylenimine Nanostructures in A431 Cells. BioImpacts 2011, 1 (1), 23-30.
(43) Fröhlich, E. The Role of Surface Charge in Cellular Uptake and Cytotoxicity of Medical Nanoparticles. Int. J. Nanomedicine 2012, 7, 5577-5591.
(44) Malyala, P.; Singh, M. Endotoxin Limits in Formulations for Preclinical Research. J. Pharm. Sci. 2008, 97 (6), 2041-2044.
(45) Goldenthal, K. L.; Pastan, I.; Willingham, M. C. Initial Steps in Receptor-Mediated Endocytosis. The Influence of Temperature on the Shape and Distribution of Plasma Membrane Clathrin-Coated Pits in Cultured Mammalian Cells. Exp. Cell Res. 1984, 152 (2), 558-564.
(46) Wang, L. H.; Rothberg, K. G.; Anderson, R. G. W. Mis-Assembly of Clathrin Lattices on Endosomes Reveals a Regulatory Switch for Coated Pit Formation. J. Cell Biol. 1993, 123 (5), 1107-1117.
(47) Nabi, I. R.; Le, P. U. Caveolae/Raft-Dependent Endocytosis. J. Cell Biol. 2003, 161 (4), 673-677.
(48) Koivusalo, M.; Welch, C.; Hayashi, H.; Scott, C. C.; Kim, M.; Alexander, T.; Touret, N.; Hahn, K. M.; Grinstein, S. Amiloride Inhibits Macropinocytosis by Lowering Submembranous PH and Preventing Rac1 and Cdc42 Signaling. J. Cell Biol. 2010, 188 (4), 547-563.
(49) Kline, S. R. Reduction and Analysis of SANS and USANS Data Using IGOR Pro. J. Appl. Crystallogr. 2006, 39 (6), 895-900.
(50) Percus, J. K.; Yevick, G. J. Analysis of Classical Statistical Mechanics by Means of Collective Coordinates. Phys. Rev. 1958, 110 (1), 1-13.
(51) Volinia, S.; Calin, G. A.; Liu, C. G.; Ambs, S.; Cimmino, A.; Petrocca, F.; Visone, R.; Iorio, M.; Roldo, C.; Ferracin, M.; Prueitt, R. L.; Yanaihara, N.; Lanza, G.; Scarpa, A.; Vecchione, A.; Negrini, M.; Harris, C. C.; Croce, C. M. A MicroRNA Expression Signature of Human Solid Tumors Defines Cancer Gene Targets. Proc. Natl. Acad. Sci. U.S.A 2006, 103 (7), 2257-2261.
(52) Mattiske, S.; Suetani, R. J.; Neilsen, P. M.; Callen, D. F. The Oncogenic Role of MiR-155 in Breast Cancer. Cancer Epidemiol. Biomarkers Prev. 2012, 21 (8), 1236-1243.
(53) Schetter, A. J.; Leung, S. Y.; Sohn, J. J.; Zanetti, K. A.; Bowman, E. D.; Yanaihara, N.; Yuen, S. T.; Chan, T. L.; Kwong, D. L. W.; Au, G. K. H.; Liu, C. G.; Calin, G. A.; Croce, C. M.; Harris, C. C. MicroRNA Expression Profiles Associated with Prognosis and Therapeutic Outcome in Colon Adenocarcinoma. JAMA—J. Am. Med. Assoc. 2008, 299 (4), 425-436.
(54) Zhang, W.; Wei, L.; Luo, R.; Liu, H.; Chen, J. The Value of MicroRNA-21 as a Biomarker for the Prognosis of Lung Cancer: A Protocol for Systematic Review and Meta-Analysis. Medicine (Baltimore). 2020, 99 (33), e21483.
(55) Kluiver, J.; Poppema, S.; de Jong, D.; Blokzijl, T.; Harms, G.; Jacobs, S.; Kroesen, B. J.; van den Berg, A. BIC and MiR-155 Are Highly Expressed in Hodgkin, Primary Mediastinal and Diffuse Large B Cell Lymphomas. J. Pathol. 2005, 207(2), 243-249. https://doi.org/10.1002/path.1825.
(56) Metzler, M.; Wilda, M.; Busch, K.; Viehmann, S.; Borkhardt, A. High Expression of Precursor MicroRNA-155/BIC RNA in Children with Burkitt Lymphoma. Genes Chromosom. Cancer 2004, 39 (2), 167-169.
(57) Babar, I. A.; Cheng, C. J.; Booth, C. J.; Liang, X.; Weidhaas, J. B.; Saltzman, W. M.; Slack, F. J. Nanoparticle-Based Therapy in an in vivo MicroRNA-155 (MiR-155)-Dependent Mouse Model of Lymphoma. Proc. Natl. Acad. Sci. U.S.A 2012, 109 (26).
(58) Torchilin, V. Tumor Delivery of Macromolecular Drugs Based on the EPR Effect. Adv. Drug Deliv. Rev. 2011, 63 (3), 131-135. https://doi.org/10.1016/j.addr.2010.03.011.
(59) Samal, S. K.; Dash, M.; Vlierberghe, S. Van; Kaplan, D. L.; Chiellini, E.; Blitterswijk, C. van; Moroni, L.; Dubruel, P. Cationic Polymers and Their Therapeutic Potential. Chem. Soc. Rev. 2012, 41 (21), 7147-7194.
(60) Wahane, A.; Waghmode, A.; Kapphahn, A.; Dhuri, K.; Gupta, A.; Bahal, R. Role of Lipid-Based and Polymer-Based Non-Viral Vectors in Nucleic Acid Delivery for next-Generation Gene Therapy. Molecules 2020, 25 (12), 2866.
(61) Lv, H.; Zhang, S.; Wang, B.; Cui, S.; Yan, J. Toxicity of Cationic Lipids and Cationic Polymers in Gene Delivery. J. Control. Release 2006, 114 (1), 100-109.
(62) Fields, R. J.; Cheng, C. J.; Quijano, E.; Weller, C.; Kristofik, N.; Duong, N.; Hoimes, C.; Egan, M. E.; Saltzman, W. M. Surface Modified Poly(β Amino Ester)-Containing Nanoparticles for Plasmid DNA Delivery. J. Control. Release 2012, 164 (1), 41-48
(63) Fields, R. J.; Quijano, E.; Mcneer, N. A.; Caputo, C.; Bahal, R.; Anandalingam, K.; Egan, M. E.; Glazer, P. M.; Saltzman, W. M. Modified Poly(Lactic-Co-Glycolic Acid) Nanoparticles for Enhanced Cellular Uptake and Gene Editing in the Lung. Adv. Healthc. Mater. 2015, 4 (3), 361-366.
(64) Lee, E. S.; Shin, H. J.; Na, K.; Bae, Y. H. Poly(L-Histidine)-PEG Block Copolymer Micelles and PH-Induced Destabilization. J. Control. Release 2003, 90 (3), 363-374.
(65) L1, Z.; Qiu, L.; Chen, Q.; Hao, T.; Qiao, M.; Zhao, H.; Zhang, J.; Hu, H.; Zhao, X.; Chen, D.; Mei, L. PH-Sensitive Nanoparticles of Poly(L-Histidine)-Poly(Lactide-Co-Glycolide)-Tocopheryl Polyethylene Glycol Succinate for Anti-Tumor Drug Delivery. Acta Biomater. 2015, 11 (1), 137-150.
(66) Beck-Broichsitter, M.; Rytting, E.; Lebhardt, T.; Wang, X.; Kissel, T. Preparation of Nanoparticles by Solvent Displacement for Drug Delivery: A Shift in the “Ouzo Region” upon Drug Loading. Eur. J. Pharm. Sci. 2010, 41 (2), 244-253.
(67) He, J.; Xu, S.; James Mixson, A. The Multifaceted Histidine-Based Carriers for Nucleic Acid Delivery: Advances and Challenges. Pharmaceutics 2020, 12 (8), 1-31.
(68) Malik, S.; Lim, J.; Slack, F. J.; Braddock, D. T.; Bahal, R. Next Generation MiRNA Inhibition Using Short Anti-Seed PNAs Encapsulated in PLGA Nanoparticles. J. Control. Release 2020, 327 (March), 406-419.
(69) Malik, S.; Slack, F. J.; Bahal, R. Formulation of PLGA Nanoparticles Containing Short Cationic Peptide Nucleic Acids. Methods X 2020, 7, 101115.
(70) Dawidczyk, C. M.; Russell, L. M.; Hultz, M.; Searson, P. C. Tumor Accumulation of Liposomal Doxorubicin in Three Murine Models: Optimizing Delivery Efficiency. Nanomedicine Nanotechnology, Biol. Med. 2017.

### Example 10. Poly-Histidine Formulations for miRNA Mimic Delivery

Two forms of miRNA-based therapy can be used in the miRNA dysregulation in disease: antimiRs and miRNA mimics. miRNA mimics are synthetic double stranded nucleic acids that mimic the activity of endogenous miRNAs through the activation of the RISC complex by the Argonaute 2 protein (7). The synthetic nucleic acid design consists of a double stranded structure with both the active and passenger strand. Upon entering the cytoplasm and activation of RISC, the passenger strand degrades and the miRNA active strand binds to the target RNA strand through Watson-Crick base pairing (8). Given the negative charge of the miRNA mimics and the potential for enzymatic degradation because of its double stranded structure, successful delivery of these oligonucleotides is not without challenges. Charge is important for delivery as a cationic delivery system would give better loading of miRNA mimics than negatively charged carriers. However, delivery systems that possess high positive charge are too toxic for translation to the clinic. Off-target delivery also has the potential to have undesired side effects and toxicity due to accumulation in specific organs,

The present inventors have developed a proof-of-concept for miRNA mimic delivery where the inventors show efficacy on both in vitro and in vivo fronts by nanoparticle delivery of miRNA mimics using the cationic delivery system as described in the Examples above. Briefly, a polymeric nanocarrier, poly-lactic-co-glycolic acid nanoparticle with poly-L-Histidine patches on the surface was used to deliver both peptide nucleic acids and small molecules such as paclitaxel. These nanoformulations were made using a double emulsion solvent evaporation technique with Acetone:Dichloromethane (2:1 v/v) as the solvent system, resulting in small particle size and uniform size distribution. PLGA-poly-L-Histidine nanoparticles showed proficient cellular uptake and reduced tumor growth in vivo with the delivery of paclitaxel. The presence of histidine on the surface gives the nanoparticle a cationic charge due to an imidazole ring in histidine residues. This could result in optimal loading of negatively charged miRNA mimics from a formulation standpoint.

To test this delivery platform for miRNA mimics, miR-34a mimics were used for proof-of-concept. miR-34a is a potent tumor suppressor miRNA that is downregulated in many solid tumors including lung adenocarcinomas (10,11). miR-34a is involved in inhibiting a variety of cancer-causing pathways including the epithelial to mesenchymal transition state (12). In the mesenchymal state, cells are more migratory and invasive leading to angiogenesis and tumor progression (13). This can be combatted through the delivery of miR-34a mimics to increase miR-34a activity with the desired target response. However, the translation of the miRNA mimic technology to the clinic has remained a challenge. In 2013, Mirna Therapeutics, now Synlogic, developed a liposomal-miRNA mimic formulation to target miR-34a. MRX34 was eventually tested in Phase I clinical trials (14). This formulation uses an ionic liposome to encapsulate miR-34a mimics. However, the trial was eventually halted as patients were facing serious adverse events such as cytokine release, hypoxia, and hepatic failure (15). Therefore, there is a need for a safe and biocompatible nanocarrier for the delivery of miRNA mimics.

miRNA mimics are a promising technology that has therapeutic potential to treat numerous diseases that are caused by depleted levels of specific miRNAs. In this Example, it was established that PLGA-poly-L-Histidine nanoparticles re a potential nanocarrier to successfully deliver miRNA mimics both in vitro and in vivo. Thorough biophysical characterization confirmed the stability of the nanoparticle formulation. SAXS revealed the structural arrangement of miRNA mimics within the PLGA-poly-L-His NPs. miR-34a loaded NPs were tested in cell culture in the A549 cell line where cellular uptake and route of endocytosis were evaluated. miR-34a and p53 levels were also evaluated using RT-PCR and Western blot analysis. Cell viability assay and apoptosis assays were used to analyze the extent of cell survival when treated with miR-34a NPs. In vivo efficacy was also demonstrated by testing the miR-34a NPs intratumorally in A549 xenograft mice. The results of these studies show promise in development of miRNA mimic therapeutics while utilizing a safe and effective delivery system.

### Results

miR-34a NP Formulations and Physicochemical Characterization

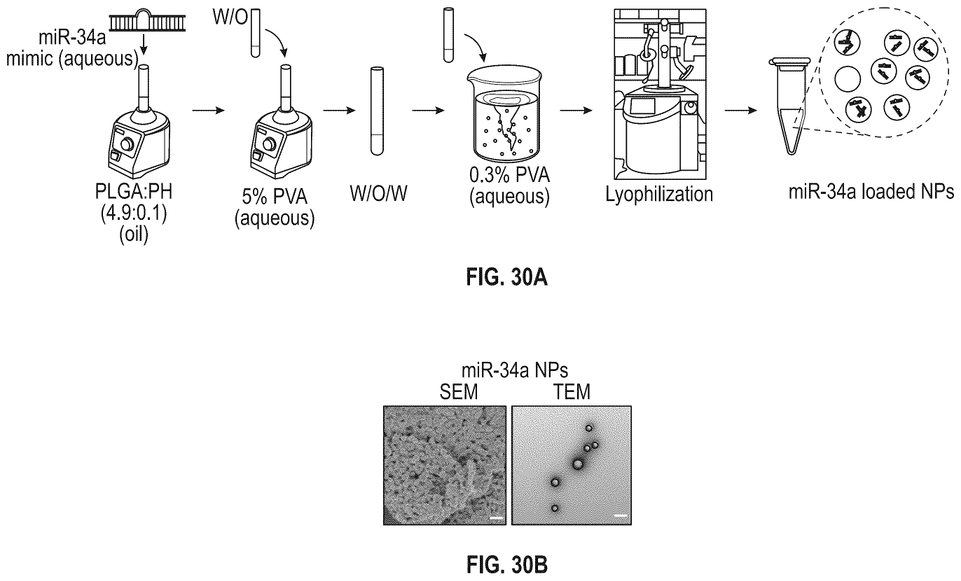

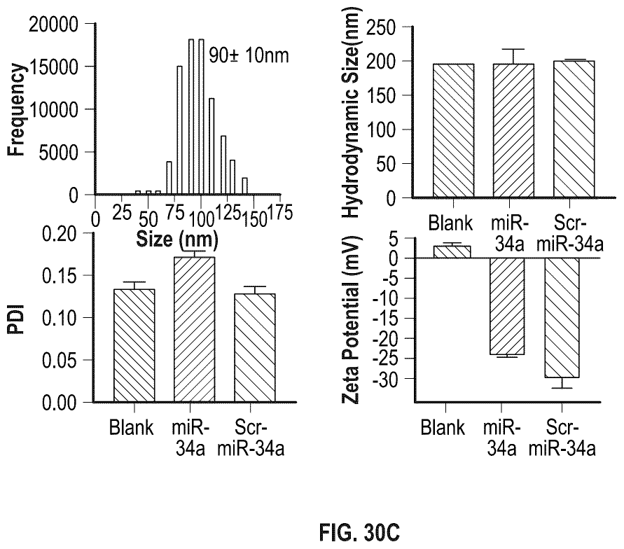

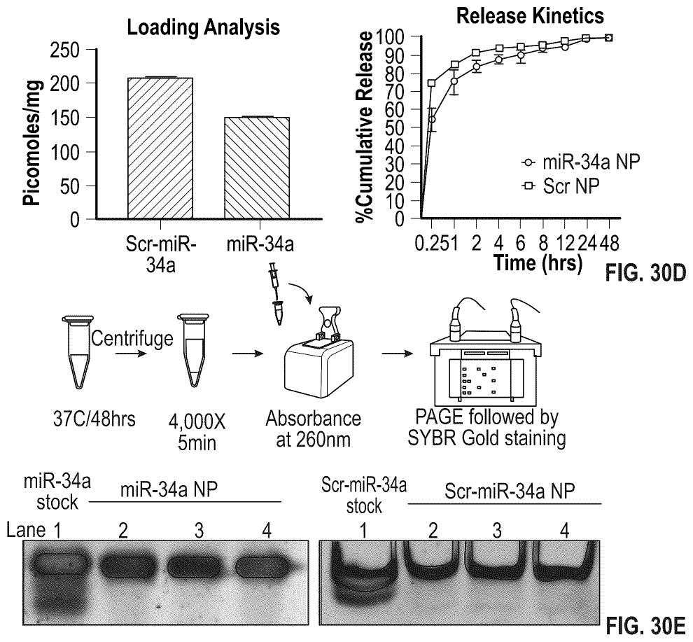

As the delivery of miRNA mimics is a challenge due to potential for degradation, a nanoparticle delivery system was utilized to circumvent stability issues present with oligonucleotide delivery. PLGA-poly-Histidine nanoparticles of the present invention are a unique cationic delivery system, where a PLGA nanoparticle core contains patches of histidine residues on the surface to give the particles cationic charge that can be fine-tuned based on the amount of histidine present during formulation. A double emulsion solvent evaporation technique was used for nanoparticle synthesis to load peptide nucleic acids and paclitaxel. The PLGA:poly-L-His w/w ratio was deemed optimal at 4.9:0.1 as the positive charge was not too high to cause toxicity. In this study, miRNA mimics were tested with the same delivery system as the cationic delivery system would improve loading of the miRNA mimics (FIG. 30A). Initially, PLGA NPs were tested but did not exhibit the same loading as with PLGA-poly-L-His NPs. 3 formulations were synthesized using the same double emulsion evaporation technique: Blank NPs (without miRNA mimic loaded), miR-34a NPs, and Scr-34a NPs (NPs containing scramble mimic sequence). A fourth formulation (miR-34a-FITC NPs) was used containing miR-34a mimics covalently conjugated with FITC for cellular uptake studies.

Physicochemical properties including morphology, particle size and distribution, and surface charge of the formulations were tested using scanning/transmission electron microscopy and dynamic light scattering. Morphology of Blank NPs and miR-34a NPs were spherical and uniform as shown by SEM images (FIG. 30B). In addition, TEM images reveal spherical morphology in solution state and stable morphological structure of NPs when encapsulated with miR-34a mimics. The hydrodynamic size and polydispersity were measured using dynamic light scattering. Dynamic light scattering is used to quantify the Brownian motion of nanoparticles in a solvent such as water, termed as hydrodynamic size (18). The hydrodynamic size was about 200 nm for each formulation (Blank NPs, miR-34a NPs and Scr-34a NPs) with a polydispersity index (PDI) of 0.10-0.15 for each formulations (FIG. 30C). The surface charge was measured in Zeta potential (mV) and was +2 mV for Blank NPs, −20 mV for miR-34a NPs, and −26 mV for Scr-34a NPs (FIG. 30C). This shift from a positive to negative charge when loaded with the miRNA mimic confirms the successful loading of the mimic in the formulation.

### Loading and Release Kinetics of Mimic-Loaded NPs

Next, to understand the quantity of miRNA mimic that is present in the NP formulation as well how much mimic was released by time, the loading and percent cumulative release were quantified for both miR-34a and Scr-34a NPs. For the loading study, the mimic was extracted by using dichloromethane to break down the polymer and mimic was extracted using aqueous buffer. The absorbance was taken at 260 nm using Nanodrop and loading in picomoles/mg was calculated. The loading was calculated to be 150-200 picomoles/mg for miR-34a and Scr-34a NPs (FIG. 30D). The release of mimic was quantified over a 48 hr time-period. An initial burst release was observed at 15 min followed by 50-70% release by 1 hr, and overall complete release by 24 hrs (FIG. 30D).

RNA Integrity of miR-34a Mimic in NP Formulation

To test the stability of the miR-34a mimics in our formulation, an in vitro release assay of the miR-34a NPs was performed. Both miR-34a and Scr-34a NPs were resuspended in PBS at 37′C for 48 hrs to release the mimic from the formulation and loaded the released mimic into a 5% PAGE gel to evaluate RNA stability. A 5% PAGE gel was loaded with a 1 μM mimic stock as a control and the released mimic was also loaded in the remaining wells. This study was performed using samples from three different nanoparticle batches of both miR-34a and Scr-34a NP formulations. The PAGE gel reveals the stability of both miR-34a and Scr-34a mimics by the presence of similar band intensity between the mimic stock in Lane 1 and the released mimic in Lanes 2-4 (FIG. 30E).

Structural Characterization of miR-34a NPs Using Small Angle X-Ray Scattering

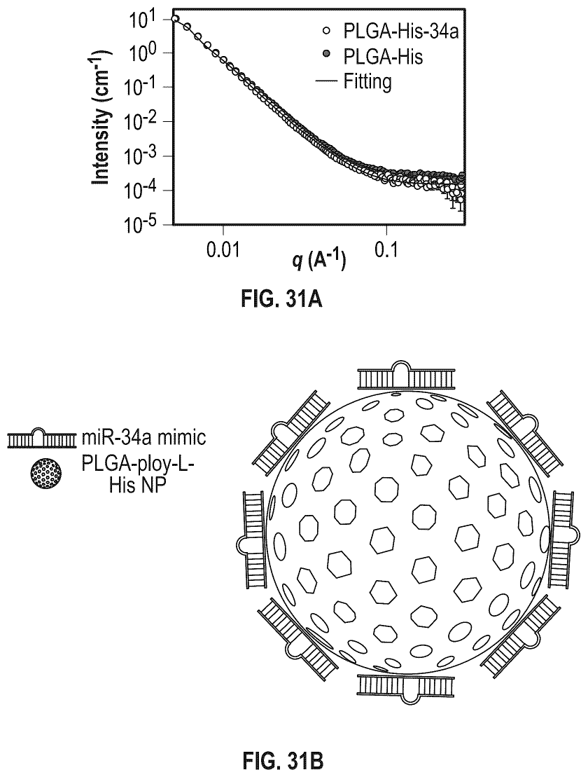

The PLGA-poly-L-His NPs has a core-shell spherical structures. The same morphology was adopted to fit the SAXS data of PLGA-poly-L-His NPs in the absence and presence of miR-34a mimics as shown in FIG. 31A to understand how miR-34a associated with the NPs. The low q regime for both samples shows q⁻⁴ decays indicating that the particle size is larger than 800 Å which is outside the SAXS probing range. The subtle difference in high q regime (>0.1 Å-1), where higher scattering intensity and slower decay of NP containing miR-34a mimics than that of Blank NPs are observed, suggests structural variation at a length scale smaller than 60 Å. Table 2 illustrates the best fitting parameters (using core-shell spherical model) which show the most significant difference to be the shell thickness (T) varying from 98 (±1) Å (in the absence of miR-34a) to 106 (±1) Å after the association with miR-34a mimics. This can be attributed to the binding of miR-34a onto the surface of PLGA-poly-L-His NPs as shown in FIG. 31B.

TABLE 2
The best fitted results from the core-
shell spherical model with a power law.

| Parameters | PLGA-His | PLGA-His-34a |
|---|---|---|
| Core Radius (R_{c}) (Å) | >800 | >800 |
| Thickness (T) (Å) | 98 ± 1 | 106 ± 1 |
| ρ_{core} (×10⁻⁶ Å⁻²) | 8.3 ± 0.03 | 8.2 ± 0.03 |
| ρₛₕₑₗₗ (×10⁻⁶ Å⁻²) | 8.5 ± 0.02 | 8.4 ± 0.02 |

Cellular Uptake of miR-34a NPs

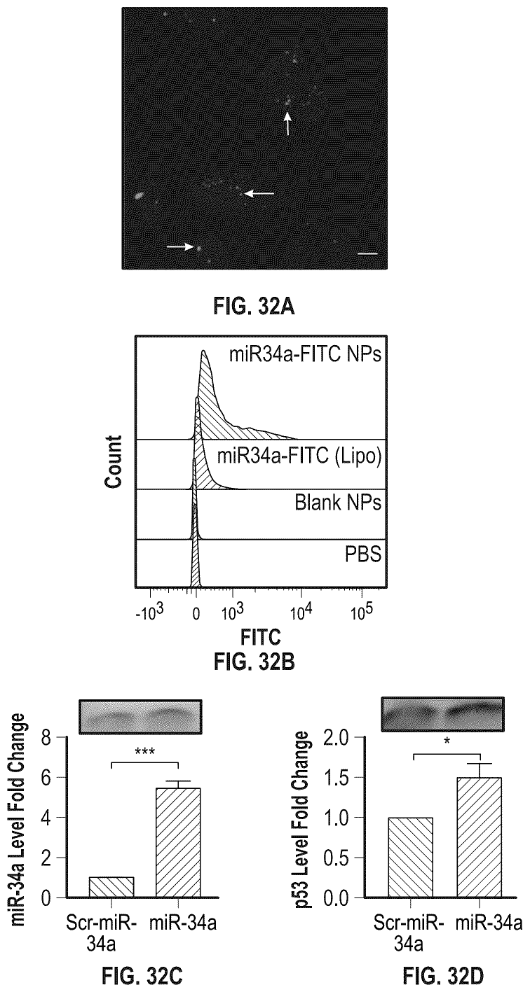

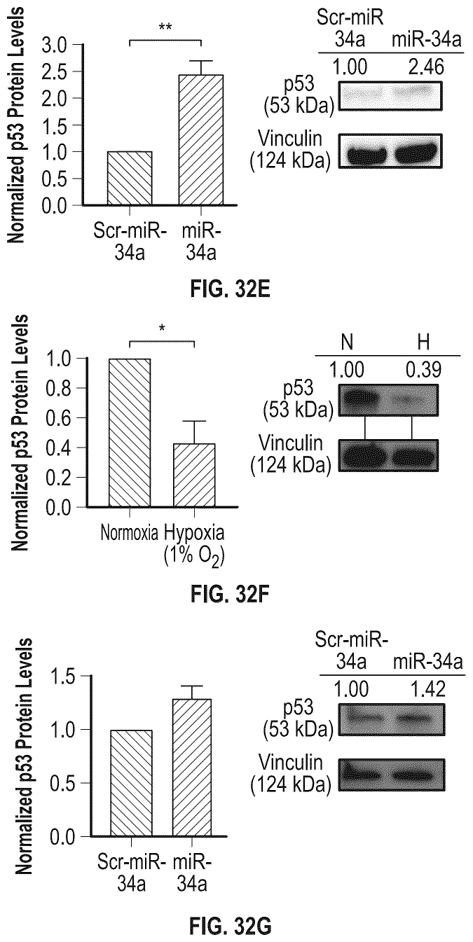

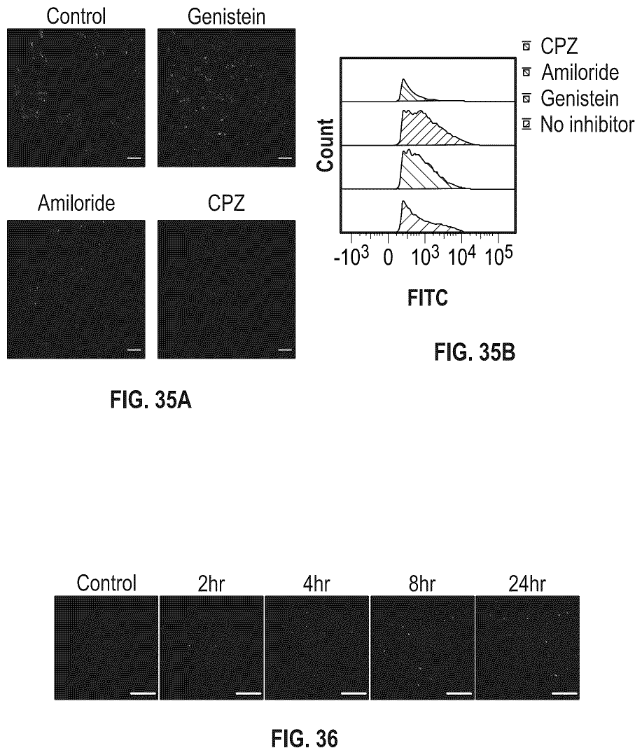

After understanding the physicochemical properties of the NP formulation, the formulation was tested in cell culture in A549 cells. This cell line is a lung adenocarcinoma cell line where miR-34a and p53 levels are reduced, causing cell proliferation (12). Initially, the distribution of the formulation in the cells was observed. miR-34a mimic covalently conjugated with FITC fluorophore was loaded into the NPs for the cellular uptake and endocytosis studies. A549 cells were treated with the miR-34a-FITC NPs for 24 hrs at a 2 mg/ml NP dose as PLGA-poly-L-His NPs show strong efficacy when loaded with peptide nucleic acids and paclitaxel at this NP dose. Cellular uptake was analyzed using confocal microscopy and then quantified in a separate study with the same treatment conditions using flow cytometry. At a 2 mg/ml dose, high cellular uptake was observed as the nanoparticles are localized near the nucleus and distributed in the cytoplasm (FIG. 32A). Quantification by flow cytometry also confirms a higher FITC signal when treated with miR-34a-FITC NPs when compared with Lipofectamine delivered miR-34a-FITC at an equivalent dose (FIG. 32B). Using confocal microscopy and flow cytometry, the route of endocytosis was also investigated using endocytosis inhibitors (FIG. 35A and FIG. 35B). Chlorpromazine inhibits clathrin-mediated endocytosis, genistein inhibits caveolae-mediated endocytosis, and amiloride inhibits the micropinocytosis (19). It was observed that less uptake in cells treated with chlorpromazine, thus it was concluded that miR-34a NPs most likely undergo clathrin-mediated endocytosis. This was also observed using flow cytometry as less FITC signal was seen in cells treated with chlorpromazine when compared to other inhibitors meaning that the clathrin-mediated endocytosis is being inhibited (FIG. 35A and FIG. 35B). Cellular uptake was visualized over time using confocal microscopy to understand the uptake kinetics of miR-34a NPs in cells (FIG. 36). Here, a substantial increase in uptake was observed at 8 hrs of incubation (FIG. 36).

Gene-Expression Analysis when Treated with miR-34a NPs

Next, various gene expression studies confirmed the activation of miR-34a and p53 with the miR-34a NP formulation through a mechanistic approach. p53 was the downstream target of choice as it functions as a major tumor suppressor gene that is directly associated with miR-34a in a positive feedback loop (20). A549 cells were treated with Scr-34a NPs and miR-34a NPs. After 24 hrs, there is a statistically significant 5-fold increase in miR-34a levels when treated with miR-34a NPs when compared to Scr-34a NPs (FIG. 32C). The expected upregulation of p53 through the positive feedback loop was also observed. p53 levels increased by 50% when treated by miR-34a NPs, which showed statistical significance (FIG. 32D).

### p53 Protein Expression in A549 Cells

After assessing p53 levels through gene expression, the protein level was also determined by Western blot. Vinculin was used as the endogenous control and blots were normalized to Scr-34a NPs. After 24 hrs there is a 2.46 fold increase in p53 protein levels in miR-34a NP treated cells, confirming efficacy of the treatment (FIG. 32E). Hypoxic conditions have been found to lower p53 levels in A549 cells, contributing to tumor proliferation (21). p53 protein levels were investigated under 1% 02 hypoxic conditions. Baseline p53 protein levels in hypoxia were reduced by 60% in hypoxia when compared to normal incubation conditions (FIG. 32F). To see if p53 levels are able to be further activated by miR-34a NPs in hypoxic conditions, cells were treated with miR-34a NPs in hypoxia for 24 hrs. Using Western blot to quantify p53 expression after 24 hrs, a 25% increase of p53 protein levels was observed compared to Scr-34a NPs (FIG. 32G). This equates to about an 80% increase of p53 protein from the baseline levels in A549 cells.

Apoptosis of A549 Cells when Treated with miR-34a NPs

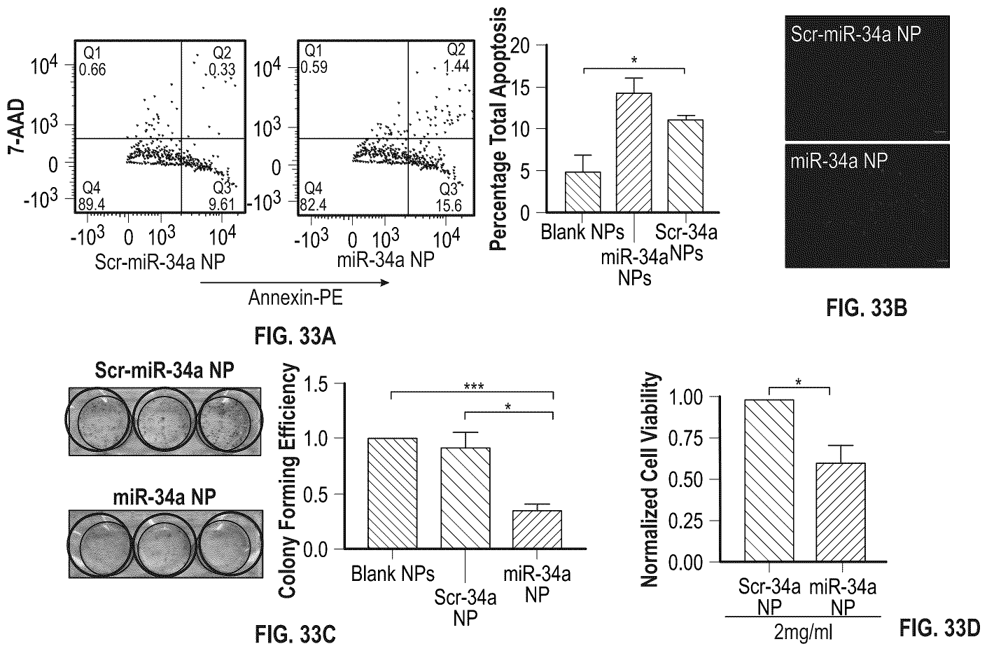

As the primary goal of miR-34a is to regulate genes that control apoptosis and control cell growth, an Annexin-V apoptosis assay through flow cytometry and fluorescence microscopy was utilized to see if the miR-34a NP treatment was effective in inducing apoptosis or necrosis (FIG. 33A). Cells were treated with Annexin-V which binds to phosphatidylserine on the cell membrane of apoptotic cells and 7-AAD, which stains necrotic cells. At a 2 mg/mL NP dose, about 15% of the total cell population was in the apoptotic state compared with only about 10% when treated with Scr-34a NPs. When using fluorescence microscopy with the same treatment conditions, there are more cells in the apoptotic state, indicated by green puncti, when treated with miR-34a NPs when compared to Scr −34a NP treatment (FIG. 33B).

Colony Forming Efficiency when Treated with miR-34a NPs

Reduced cell viability of cancer cells is expected through the effective activation of miR-34a with our NP formulation (22). Testing the colony forming efficiency of cancer cell lines provides indication of whether the NP treatments are causing reduced proliferation. When A549 cells were treated with miR-34a NPs, the colony survival was reduced by 60% when compared to Scr-34a NP treatment, indicated by the presence of less colonies in the cell culture plate (FIG. 33C).

### Cell Viability Using Trypan Blue Assay

To further confirm this reduction in cell viability, the trypan blue assay was also performed. After 24 hrs of treatment with Scr-34a NPs and miR-34a NPs at 2 mg/ml. A decrease in cell viability by approximately 40% was observed after 24 hrs of treatment of miR-34a NPs, with statistical significance (FIG. 33D).

In Vivo Intratumoral Efficacy of miR-34a NPs

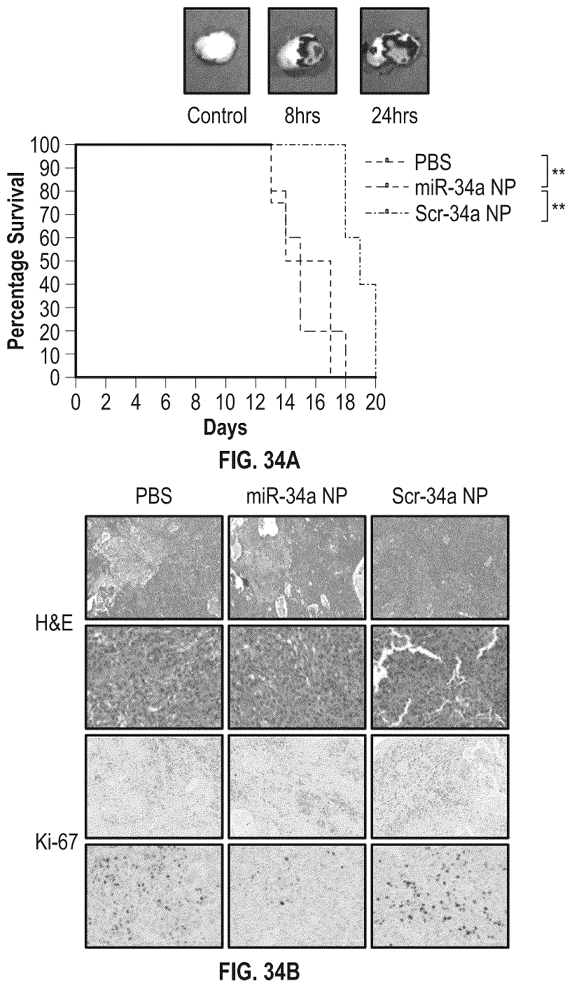

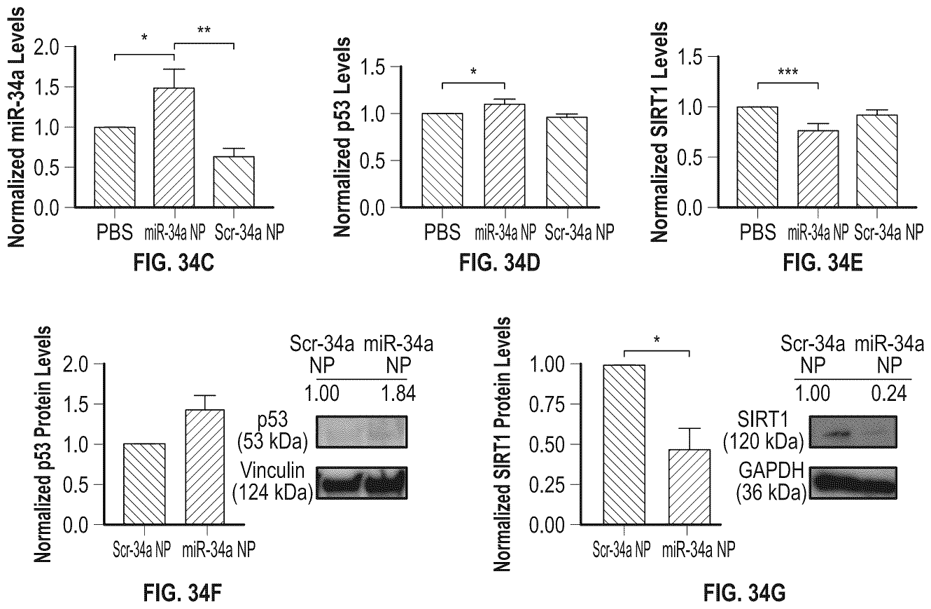

To confirm the effect of miR-34a NPs in vivo, the formulation was tested in A549 xenograft mice. A549 cells were colonized and then implanted in immunocompromised NSG mice. Tumors were allow to grow to 150 mm³. The tumor-bearing mice were then injected with PBS, miR-34a NP, or Scr-34a NPs. Survival was plotted based on when tumors reached a size of 2,000 mm³. Average survival of mice when treated with PBS was 17 days. Compared to the Scr-34a NP treatment group, survival of mice treated with miR-34a NPs was prolonged by 2 days demonstrated by an overall survival of 20 days, which was statistically significant (FIG. 34A). The intratumoral biodistribution of the miR-34a NPs was also evaluated at 8 hrs and 24 hrs to confirm the presence of the miR-34a NPs in the tumor over time. Mice were treated with miR-34a NPs labelled with FAM. In the tumor images in FIG. 34A, it was observed that mIR-34a was present in the tumor at both 8 hrs and 24 hrs, which gives good indication of tumor retention of the formulation. RNA was extracted from tumor samples to assess miR-34a, p53, and SIRT1 levels using gene expression analysis. miR-34a levels were increased by 50% along with a 25% increase in p53 and 25% downregulation of SIRT1 (FIGS. 34C-34E). On the protein level, p53 was increased by 1.5-fold and SIRT1 was decreased by 0.60 fold by Western blot analysis (FIGS. 34F-34G).

In Vivo Efficacy of miR-34a NPs Through Systemic Route of Delivery

After observing miR-34a activation in A549 xenograft tumors through intratumoral injection, the efficacy of the NPS was evaluated after the NPs were delivered systemically in A549 xenograft mice. A549 cells were colonized and implanted into NSG immunocompromised mice and treated retroorbitally with PBS, Scr-34a and miR-34a NPs when the tumors reached 150-200 mm3. Survival was plotted based on when tumors reached a size of 2,000 mm³. In a sample size of n>6 mice, miR-34a NP treated mice exhibited significantly prolonged survival by 4-5 days when compared to Scr-34a NP and PBS treated mice.

### Discussion

Noncoding RNAs are a newly discovered category of RNAs that have shown to play a role in the onset many diseases by regulating gene expression. The noncoding RNA class is broken down into different subgroups by length and function. miRNAs are shorter RNA sequences (˜23 bp) whereas lncRNA and circRNA can be >100 base pairs in length (23). lncRNAs and circRNAs regulate gene expression through RNA splicing and chromatin regulation (23). miRNAs on the other hand, activate RISC to induce RNA degradation. This Example focused on targeting miRNAs as miRNA dysregulation is a cause of many diseases including cancer.

To target aberrantly upregulated miRNAs, oligonucleotides can be designed complementary to a miRNA sequence to block the miRNA from binding to its target RNA sequence. AntimiRs bind to the target miRNA through Watson-Crick base pairing and block the miRNA activity through steric hindrance. A number of miRNA inhibitor drugs are being tested in preclinical and clinical studies.

On the other hand, miRNA mimics are an effective way of replenishing miRNAs that are downregulated in diseases such as cancer by mimicking endogenous miRNA activity. However, the delivery of miRNA mimics has remained a challenge due to a variety of factors including possessing negative charge that can cause reduced uptake and low payload. Another important shortcoming to consider is the double stranded structure that can trigger cytokine release leading to toxicity (27). A cationic delivery system can improve the loading of negatively charged payloads. However, this does not exist without challenges of its own as highly positively charged delivery systems are known to cause systemic toxicity in vivo. Delivery systems with the ability to tune the positive charge would be beneficial to reduce potential for toxicity. Liposomal delivery had been a promising route for delivery of miRNA mimics (28). Amphoteric liposomes were used to deliver miR-34a systemically a for liver cancer (28). Although this was successful in preclinical settings, there were adverse events reported when liposomal delivery was used in humans in Phase I clinical trials (14). Charged lipids have shown to induce toxicity-related symptoms. For the MRX34 clinical trial these side effects included liver failure, hypoxia, and cytokine release which can all be attributed to the use of charged lipids and off-target delivery. Although lipids help to alleviate issues with loading and encapsulating miRNA payloads, the cost associated with this is unwanted side effects. Furthermore, scale-up of liposomal formulations containing nucleic acids is a challenge as observed in lipid-based vaccines. Using a polymeric nanodelivery system is a safer alternative due to the natural breakdown of the polymer when administered. PLGA is an FDA approved, biodegradable polymer that has shown to be effective in delivery of nucleic acids (29). For the delivery of negatively charged miRNA mimics, the use of a cationic delivery system is optimal to improve loading through ionic interaction.

The present invention provides a novel method using polymeric nanoparticles to effectively deliver miR-34a mimics as a proof-of-concept for miRNA mimic delivery. In the current studies, the use of PLGA-poly-L-His nanoparticles was shown to be beneficial for the delivery of miR-34a, standing as a proof-of-concept for the delivery of other miRNA mimics for the treatment of other diseases. This delivery system has also been shown to exhibit strong cellular uptake properties when loaded with peptide nucleic acids and paclitaxel. Through endocytosis inhibitor studies, it was established that miRNA mimic loaded NPs undergo clathrin-mediated endocytosis. The efficacy was confirmed in vitro through thorough gene expression and western blot analysis to corroborate the functional activity of the formulation. Both miR-34a and its target tumor suppressor transcription factor, p53, were activated on both the gene and protein level. This is accompanied by a significant reduction in the A549 cell survival through increased apoptosis as shown by Annexin-V based apoptosis assays. The efficacy was also confirmed on the in vivo front as the survival of A549-derived xenograft mice were prolonged via intratumoral injection. This was accompanied by an increase of miR-34a and p53 levels. Expression of SIRT1, which is responsible for regulating p53 deacetylation (30), was also explored, and a significant inhibition of SIRT1 was shown, confirming the suppression of A549 cell proliferation in vivo.

Understanding the structural characterization of miRNA mimics within the PLGA-poly-L-His NPs, using SAXS, allows to fine-tune the NP formulation to increase the payload. The SAXS patterns show increased shell thickness of NPs with the association of miRNA compared to the Blank NPs, suggesting that the miRNA mimics presumably coat on the surface of the nanoparticle agreeing with the fact of a fast release profile.

Studies have shown that other oligonucleotides such as peptide nucleic acids undergo exocytosis (31). Although the endocytosis of miRNA mimics was investigated, the mechanism of exocytosis has not been explored. This mechanistic understanding would shed light on the amount of miRNA mimic present and the amount that becomes excreted out of the cells over time. To this end miRNA mimic-loaded exosomes could be derived as another delivery platform. Exosomes are versatile in nature and are produced by cellular processes and can undergo a variety of uptake mechanisms including membrane fusion and phagocytosis (32).

In addition to investigating cellular trafficking, the stability of the mimic in systemic delivery must be explored given the structural arrangement of the formulation. miRNA mimics are prone to enzymatic degradation in systemic circulation. Improved stability can result from modifying the backbone of the miRNA mimic. Phosphorothioate modifications have shown increased longevity of antisense oligonucleotides in systemic circulation given their ability to bind to serum proteins when compared to oligonucleotides containing the phosphodiester backbone.

Nevertheless, miRNA mimics remain a new line of promising treatments in the realm of RNA therapeutics to treat a variety of disorders. In chronic obstructive pulmonary disorder (COPD), it has been established that miR-24 is downregulated and plays a strong role in the onset of this disease (33). The use of miR-24 mimics should be investigated for the treatment of COPD and has shown effective uptake in lungs when delivered using nanoparticles. Here, miR-24 functions through BIM and BRCA1, which are responsible for inducing excessive inflammation leading to emphysema by constant activation of the DNA damage response. Other miRNAs such as miR-16 has been found to be downregulated in mesothelioma and has been tested in clinical trials (34). In the same way, other mimics should be explored to target the noncoding RNAs that are responsible for causing debilitating diseases such as cancer and autoimmune disorders.

### Material and Methods

Hsa-miR-34a-5p (No: MC11030) and negative control mimics (No: MC10340) were commercially purchased from ThermoFisher Scientific. Polylactic-co-glycolic (50:50) acid-ester terminated was purchased from Lactel Absorbable Polymers at a 0.39 g/dL viscosity grade. Poly-L-Histidine was bought from Sigma-Aldrich. Organic solvents such as Acetone and Dichloromethane and cryoprotectants such as sucrose were purchased from Sigma Aldrich. For cell culture studies, A549 cells (ATCC, CCL-185) were grown in EMEM media (ATCC) at 37° C. and 5% CO2.

### Synthesis of PLGA-Poly-L-Histidine Nanoparticles

To formulate miR-34a loaded nanoparticles, a double emulsion solvent evaporation technique was used as described herein and in Wahane and Malik et al., 2021. Acetone and dichloromethane were used as the organic solvent, containing PLGA and poly-L-Histidine at a 4.9:0.1 w/w ratio in 750 mL of Acetone:DCM solution. miR-34a mimic, dissolved in water (1 mM, 1 nmol/mg), was added dropwise to the organic phase while vortexing to form a w/o emulsion. This single emulsion was then sonicated using a probe sonicate for 10 seconds in 3 pulses. The single emulsion was added to 1.5 mL of 5% w/v polyvinvyl alcohol (Sigma-Aldrich) solution to form a w/o/w double emulsion. The double emulsion was sonicated using a probe sonicator for 10 seconds in 3 pulses. The double emulsion was added dropwise while vortexing to 15 mL of 0.3% w/v polyvinyl alcohol (Sigma-Aldrich) solution. The suspension was stirred overnight at RT. The nanosuspension was then washed with water using a Beckman-Coulter Optima XPN-100 Ultracentrifuge 3 times at 20,000 rpm for 20 min cycles. After the third cycle, the resulting pellet was resuspended in a 5 mg/ml sucrose (Sigma-Aldrich) solution at a 1:1 PLGA:Sucrose w/w ratio. The nanoparticles were then lyophilized overnight. Using the same method, negative control mimics and FITC conjugated miR-34a mimics were loaded into PLGA-poly-L-His NPs

Biophysical Characterization of miR-34a NPs

Dynamic light scattering using the Zetasizer Nano ZS (Malvern Panalytical, Westborough, MA, USA) was used to measure the NP size in hydrodynamic diameter and the polydispersity index. The surface charge density was measured as zeta potential (mV). Each sample consisted of 3 measurements, of which the average was taken.

### Loading Analysis

The loading of miRNA mimics in PLGA-poly-L-His NPs was quantified by referring to a previously established method (9). This was done by adding 200 μl of DCM to lyophilized miR-34a NPs and shaking at 37° C. at 1,000 rpm for 24 hrs. After 24 hrs, 100 μl of sodium acetate buffer (pH 5.8) was added and shaken at 37° C. at 1,000 rpm for 1 hr. This formed both an organic and aqueous layer, containing the miRNA mimic. The NP tube was then centrifuged at 10,000 rpm for 5 min and the supernatant was isolated to another Eppendorf tube. The absorbance was measured at 260 nm using Nanodrop One (ThermoFisher Scientific, Waltham, MA, USA). The loading was then calculated in picomols/mg. The same procedure was followed for quantifying the loading of Negative control mimic NPs.

Release Kinetics of miR-34a NPs

The release study was done in reference to (9). 300 μl of PBS (ph 7.4) was added to miR-34a NPs and vortexed until NPs were resuspended and shaken at 300 rpm at 37° C. for 15 min. After 15 min, the NPs were centrifuged down for 10 min at 15,000 rpm and the absorbance of the supernatant was taken at 260 nm using Nanodrop One (ThermoFisher Scientific, Waltham, MA, USA). The NPs were then resuspended in 300 μl of PBS in shaken until the next time point. This was repeated for each time point thereafter (1 hr, 2 hr, 4 hr, 6 hr, 8 hr, 12 hr, 24 hr, 48 hr). The % cumulative release was plotted against time.

Scanning and Transmission Electron Microscopy of miR-34a NPs

A small amount of lyophilized NPs without cryoprotectant were placed on a double sided carbon tape and sputter coated. The images were taken at 30,000× using the FEI Nova NanoSEM 450 and was quantified using ImageJ. For transmission electron microscopy (TEM), water was added to lyophilized NPs. They were added to TEM carbon grids with 1% uranyl acetate for 5 min and a FEI Tecnai TEM was used at 80 kV for imaging.

### In Vitro RNA Release Study

miR-34a NPs were resuspended in PBS and shaken at 300 rpm at 37° C. for 48 hrs. The NPs were then centrifuged at 4,000 rpm for 5 min. The absorbance of the supernatant was taken at 260 nm using Nanodrop One and the concentration was calculated. The sample was then diluted to 1 μM and loaded in a 5% PAGE gel, followed by SYBR Gold staining (Invitrogen). The gel was imaged using the BioRad Gel-Doc imager.

Structural Characterization of miR-34a NPs

SAXS experiments were conducted by using the 16ID-LiX Beamline at the National Synchrotron Light Source II where is located at the Brookhaven National Laboratory (Upton, NY). The concentration of the samples is 4 mg/mL. The solution was loaded in a sample cell sandwiched by two mica windows with a gap of ˜2 mm and the X-ray energy was 13.5 keV. The intensity is expressed as a function of scattering vector, q defined as

$$\\ \frac{4 π}{λ} sin \frac{θ}{2} ,$$

where θ is the scattering angle and λ is the wavelength. The data cover a q range from 0.005 to 2.5 Å-1. Radial averaging and q-conversion of data were analyzed by using Jupyter Notebook (16). The background subtraction and transmission correction were performed to minimize the intensity of the hydrogen bond from water at ˜2.0 Å-1. The absolute intensity was derived through comparing against water incoherent scattering intensity Iwater as reported in literature (17).

### Cellular Uptake Using Confocal Microscopy

Using a 12-well plate, 150,000 A549 (ATCC, CCL-185) cells were seeded for 4 treatment groups and n=3 for each group. Cells were either treated with PBS, Blank NPs, miR-34a-FITC NPs, or Lipofectamine-transfected miR-34a-FITC. A 2 mg/ml NP dose was used and an equivalent 300 picomol of miR-34a-FITC was transfected with Lipofectamine using forward transfection. The cells were then washed with PBS and 1 drop of NucBlue Live ReadyProbes Reagent (Invitrogen) was added to each well in media to stain the nucleus for live-cell imaging. The plate was then incubated in 37° C. for 15 min. The plate was then imaged using the Keyence BZ-X10 Fluorescence Microscope at 10× and 40× magnification. (Keyence, Japan).

### Cellular Uptake Using Flow Cytometry

Using a 12-well plate, 150,000 A549 (ATCC, CCL-185) cells were seeded for 4 treatment groups and n=3 for each group. Cells were either treated with PBS, Blank NPs, miR-34a-FITC NPs, or Lipofectamine-transfected miR-34a-FITC. A 2 mg/ml NP dose was used and an equivalent 300 picomol of miR-34a-FITC was transfected with Lipofectamine using forward transfection. After 24 hrs, the cells were washed with PBS followed by trypsinization and then transferred to Eppendorf tubes. The cells were then centrifuged at 2,000 rpm for 4 minutes and washed with PBS. The final pellet was resuspended in 300 μl of PBS and passed through filtered FACS tubes. The cell uptake was quantified using the LSR Fortessa X-20 Cell Analyzer and FlowJo. To investigate the route of endocytosis, endocytosis inhibitors such as chlorpromazine (10 μg/mL), genistein (200 μM), and amiloride (1 mM) were used to treat A549 cells. The cells were pretreated with the inhibitors for 30 minutes and then incubated with miR-34a-FITC NPs (2 mg/mL) for 4 hrs. Flow cytometry was then performed to quantify cellular uptake. The same experimental set-up was used for imaging the cells using fluorescence microscopy, however, the cells were treated with miR-34a-FITC NPs for 8 hrs.

### RT-PCR Gene Expression Analysis

200,000 A549 cells were seeded in a 12-well plate and treated with either Scr-34a NPs, miR-34a NPs, and Lipofectamine transfected mIR-34a for 24 hrs. The cells were then pelleted down and the total RNA was extracted using a Qiagen RNeasy kit. For miR-34a expression, the cDNA synthesis kit (Invitrogen) along with RT primer for miR-34a (4331182) and U6 (001973) was used to synthesis the cDNA. The samples were incubated in specified temperature conditions of the cDNA using a Bio-Rad C1000 Touch Thermal cycler. PCR amplification was done using the TM primers, RNase free water, and Universal Mastermix II with UNG and cycled in the Bio-Rad CFX-Connect Real-Time PCR instrument. The cDNA synthesis kit was also used for cDNA synthesis when measuring p53 levels although Random RT primers were used. Samples were incubated according to the specified temperature conditions. PCR amplification was done using the Universal Mastermix with UNG, water, and p53 and GAPDH primers, where GAPDH served as the endogenous control. The amplified PCR product samples were run on a 1% agarose gel at 120V for 20 min and quantified using ImageJ.

### Western Blot Study

200,000 A549 cells were seeded in a 12-well plate and treated with either Scr-34a NPs, miR-34a NPs, and Lipofectamine transfected miR-34a for 24 hrs. Cells were then trypsinized and pelleted down at 2,000 rpm for 4 min. The proteins were extracted from the cell pellet with 1×RIPA buffer and 1× Protease inhibitor (ThermoFisher Scientific). The pellets were kept in ice and vortexed every 10 min for 3 times to dissociate the pellet. BSA standards were used to measure the concentration of each protein sample by developing a standard curve based on absorbance. 20 μg of protein was added into each well of a 4-15% Mini-Protean TGX Stain Free 50 μl gel (Bio-Rad). The protein samples were run at 200V for 40 min and transferred to a PVDF membrane at 110V for 90 min. The proteins on the membrane were blocked using 5% milk in 1× Tris-buffered saline-Tween (TBST) buffer and shaken for 1 hr. The blots were then washed with 1×TBST buffer and cut according to the molecular weight of Vinculin (124 kD) and p53 (53 kD). Vinculin was used as the endogenous control. The p53 (1:500) and Vinculin (1:1000) antibody (Cell Signaling Technology) was added to the specific blot and shaken overnight at 4° C. The secondary antibody was then added to probe for the primary antibody. The blots were then submerged in Chemiluminescent HRP substrate and imaged using a Bio-Rad ChemiDoc Imaging instrument. The band intensity was quantified using ImageJ software.

### Hypoxia Cell Culture Studies

A549 cells (ATCC) were grown in EMEM media at 37° C. For baseline p53 level studies, one 12-well plate was seeded with 200,000 cells (n=3) and placed in normoxia conditions. Another 12-well plate was seeded with 200,000 cells (n=3) and placed in hypoxia conditions (1% 02 in Nitrogen). After 24 hrs, the cells were pelleted down, and proteins were extracted for western blot analysis to measure p53 protein levels. For NP treatment, cells were treated with either Scr-34a, miR-34a NPs, or Lipofectamine-transfected miR-34a and placed in hypoxia conditions (1% 02 in Nitrogen) for 24 hrs. The cells were then pellet down and proteins were extracted for western blot analysis to measure p53 protein levels.

### Cell Viability-Trypan Blue Assay

10,000 A549 cells were treated with Blank NPs and miR-34a NPs (0.2 mg, 0.4 mg, or 0.6 mg) for 24 hrs. The cells were then stained with trypan blue dye and counted using an automated Bio-Rad cell counter.

### Cell Viability—Colonogenic Assay

A549 cells were seeded in a 24-well plate and were treated with either PBS, Blank NPs, miR-34a NPs, Scr-34a NPs, and Lipofectamine-miR-34a at a 2 mg/ml NP dose for 24 hrs. The cells in each well were then counted and from each well, 100 cells were seeded into each well of a 6 well plate and incubated for 13 days to allow cell growth of treated cells. When there were 30-50 cells per colony, the cells were then washed with PBS and fixed with 4% PFA. The PFA was washed and the wells were stained with 1 mL of 1% w/v crystal violet solution for 24 hrs. After 24 hrs, the stained was washed off with water until all residual stain was removed and the plates were allowed to dry. After drying each individual colony was counted.

### Annexin-V FACS Assay

200,000 A549 cells were seeded in a 12-well plate and treated with A549 cells were seeded in a 24-well plate and were treated with either PBS, Blank NPs, miR-34a NPs, Scr-34a NPs, and Lipofectamine-miR-34a at a 2 mg/ml NP dose for 24 hrs. The cells were trypsinized and pelleted down at 2,000 rpm for 4 min and then resuspended in Annexin V Binding Buffer. 100,000 cells were passed through the filtered FACS tube. 7.5 μl of Annexin-V-phycoerythrin (PE) and 7.5 μl of 7-amino-actinomycin (7-AAD) was added to the sample. The tubes were kept away from light for 15 min. The remaining volume was made up with Annexin-V Binding buffer to reach a total volume of 300 μl. The cells were quantified using the LSR-Fortessa X-20 instrument.

### Annexin-V FITC Apoptosis Assay

10,000 A549 cells were seeded in a 96-well plate and treated with A549 cells were seeded in a 24-well plate and were treated with either PBS, Blank NPs, miR-34a NPs, Scr-34a NPs, and Lipofectamine-miR-34a at a 2 mg/ml NP dose for 24 hrs. Annexin-V FITC was added to 1× Annexin V Binding buffer and 100 μl of the diluted stock was added to each well. The plate was kept away from light for 15 min and imaged using a Keyence fluorescence microscope at 10× magnification.

### In Vivo Intratumoral Efficacy Studies

14 Female NOD-SCID mice at 5 weeks old were purchased from Jackson Laboratories. A549 cells (ATCC) were expanded for tumor implantation. Once the plates reached confluency, they were split into two plates and so on. Each mouse was injected with 1×10⁷ cells on both flanks subcutaneously. Tumors were monitored every 2 days. Once the tumor reached 150 mm3, the mice were split into 3 groups: PBS, miR-34a NP, Scr-34a NP with n=4 in PBS and n=5 in both miR-34a and Scr-34a NP groups. PBS treated mice were injected with 80 μl intratumorally and NP treatment groups were dosed intratumorally with 3 mg NPs in 80 μl of PBS. NPs were vortexed and sonicated using a bath sonicator when resuspending the particles. Doses were given on Day 1, Day 5, Day 9, and Day 13. Tumors were measured daily and volume was calculated using the length, width, and breadth measurements. When the tumors reached 2,000 mm³, the mouse was sacrificed and tumors, heart, lungs, liver, kidneys, and spleen were extracted and harvested. Tumors were dissociated and cells were extracted. RNA was isolated for gene expression studies and cell pellets were stored for Western blot. CBC analysis was done using a Sysmex CBC analyzer. Caspase-3 and Ki-67 staining was done for tumor, liver, kidney and spleen samples.

For intratumoral biodistribution studies, 3 mice were used. Tumors were implanted subcutaneously on the right flank. Once the tumor reached ˜600 mm³, the mice were split into three treatment groups: PBS, miR-34a NP 8 hr, and miR-34a 24 hr. NP-treated mice were treated with miR-34a NPs labelled with FAM fluorophore. After the specified time point, the mice were sacrificed and the tumors, heart, lungs, kidneys, and spleen were extracted and imaged using IVIS. Tumors were cryosectioned and imaged using the Keyence microscope.

### In Vivo Systemic Efficacy Studies

For the tumor survival study, 22 Female NOD-SCID mice at 5 weeks old were purchased from Jackson Laboratories. As in the intratumoral efficacy studies, A549 cells (ATCC) were expanded for tumor implantation. Each mouse was injected with 1×10⁷ cells on the right flank subcutaneously. Once the tumor reached 150-200 mm³, the mice were split into three groups: PBS, miR-34a NP, and Scr-34a NP. There were n>7 in each treatment group. PBS treated mice were injected with 100 μl retroorbitally and NP treatment groups were dosed with 3 mg NPs in 100 μl of PBS. NPs were vortexed and sonicated using a bath sonicator when resuspending the particles. Doses were given on Day 1, Day 5, Day 8, and Day 11. Tumors were measured daily and volume was calculated using the length, width, and breadth measurements. When the tumors reached 2,000 mm³, the mouse was sacrificed and tumors, heart, lungs, liver, kidneys, and spleen were extracted and harvested. Tumors were dissociated and cells were extracted. RNA was isolated for gene expression studies and cell pellets were stored for Western blot. CBC analysis was done using a Sysmex CBC analyzer. Caspase-3 and Ki-67 staining was done for tumor, liver, kidney and spleen samples.

For systemic biodistribution studies, 3 mice were used. Tumors were implanted subcutaneously on the right flank. Once the tumor reached ˜600 mm³, the mice were split into three treatment groups: PBS, miR-34a NP 4 hr, and miR-34a 8 hr. NP-treated mice were treated with miR-34a NPs labelled with FAM fluorophore. After the specified time point, the mice were sacrificed and the tumors, heart, lungs, kidneys, and spleen were extracted and imaged using IVIS. Tumors were cryosectioned and imaged using the Keyence microscope.

### REFERENCES

1. Zogg, H., Singh, R. and Ro, S. (2022) Current Advances in RNA Therapeutics for Human Diseases. Int J Mol Sci, 23.
2. Dzierlega, K. and Yokota, T. (2020) Optimization of antisense-mediated exon skipping for Duchenne muscular dystrophy. Gene Ther, 27, 407-416.
3. Zhang, M. M., Bahal, R., Rasmussen, T. P., Manautou, J. E. and Zhong, X. B. (2021) The growth of siRNA-based therapeutics: Updated clinical studies. Biochem Pharmacol, 189, 114432.
4. Pratt, A. J. and MacRae, I. J. (2009) The RNA-induced silencing complex: a versatile gene-silencing machine. J Biol Chem, 284, 17897-17901.
5. Malik, S., Lim, J., Slack, F. J., Braddock, D. T. and Bahal, R. (2020) Next generation miRNA inhibition using short anti-seed PNAs encapsulated in PLGA nanoparticles. J Control Release, 327, 406-419.
6. Seto, A. G., Beatty, X., Lynch, J. M., Hermreck, M., Tetzlaff, M., Duvic, M. and Jackson, A. L. (2018) Cobomarsen, an oligonucleotide inhibitor of miR-155, co-ordinately regulates multiple survival pathways to reduce cellular proliferation and survival in cutaneous T-cell lymphoma. Br J Haematol, 183, 428-444.
7. Wang, Z. (2011) The guideline of the design and validation of MiRNA mimics. Methods Mol Biol, 676, 211-223.
8. O'Brien, J., Hayder, H., Zayed, Y. and Peng, C. (2018) Overview of MicroRNA Biogenesis, Mechanisms of Actions, and Circulation. Front Endocrinol (Lausanne), 9, 402.
9. Wahane, A., Malik, S., Shih, K. C., Gaddam, R. R., Chen, C., Liu, Y., Nieh, M. P., Vikram, A. and Bahal, R. (2021) Dual-Modality Poly-l-histidine Nanoparticles to Deliver Peptide Nucleic Acids and Paclitaxel for In vivo Cancer Therapy. ACS Appl Mater Interfaces, 13, 45244-45258.
10. Slabakova, E., Culig, Z., Remsik, J. and Soucek, K. (2017) Alternative mechanisms of miR-34a regulation in cancer. Cell Death Dis, 8, e3100.
11. Shi, X., Kaller, M., Rokavec, M., Kirchner, T., Horst, D. and Hermeking, H. (2020) Characterization of a p53/miR-34a/CSF1R/STAT3 Feedback Loop in Colorectal Cancer. Cell Mol Gastroenterol Hepatol, 10, 391-418.
12. Kawami, M., Takenaka, S., Akai, M., Yumoto, R. and Takano, M. (2021) Characterization of miR-34a-Induced Epithelial-Mesenchymal Transition in Non-Small Lung Cancer Cells Focusing on p53. Biomolecules, 11.
13. Zhang, L., Liao, Y. and Tang, L. (2019) MicroRNA-34 family: a potential tumor suppressor and therapeutic candidate in cancer. J Exp Clin Cancer Res, 38, 53.
14. Hong, D. S., Kang, Y. K., Borad, M., Sachdev, J., Ejadi, S., Lim, H. Y., Brenner, A. J., Park, K., Lee, J. L., Kim, T. Y. et al. (2020) Phase 1 study of MRX34, a liposomal miR-34a mimic, in patients with advanced solid tumours. Br J Cancer, 122, 1630-1637.
15. Beg, M. S., Brenner, A. J., Sachdev, J., Borad, M., Kang, Y. K., Stoudemire, J., Smith, S., Bader, A. G., Kim, S. and Hong, D. S. (2017) Phase I study of MRX34, a liposomal miR-34a mimic, administered twice weekly in patients with advanced solid tumors. Invest New Drugs, 35, 180-188.
16. Yang, L. (2013) Using an in-vacuum CCD detector for simultaneous small- and wide-angle scattering at beamline X9. Journal of Synchrotron Radiation, 20, 211-218.
17. Fan, L., Degen, M., Bendle, S., Grupido, N. and Ilavsky, J. (2010) The Absolute Calibration of a Small-Angle Scattering Instrument with a Laboratory X-ray Source. Journal of Physics: Conference Series, 247, 012005.
18. Babick, F. (2020) In Hodoroaba, V.-D., Unger, W. E. S. and Shard, A. G. (eds.), Characterization of Nanoparticles. Elsevier, pp. 137-172.
19. Chang, C. C., Wu, M. and Yuan, F. (2014) Role of specific endocytic pathways in electrotransfection of cells. Mol Ther Methods Clin Dev, 1, 14058.
20. Yamakuchi, M. and Lowenstein, C. J. (2009) MiR-34, SIRT1 and p53: the feedback loop. Cell Cycle, 8, 712-715.
21. Hong, H., Zhenxiang, Z., Youngjian, X. and Jingfang, S. (2003) Expression of p53, p21 in human lung adenocarcinoma A549 cell strains under hypoxia conditions and the effect of TSA on their expression. Journal of Huazhong University of Science and Technology [Medical Sciences], 23, 359-361.
22. Rui, X., Zhao, H., Xiao, X., Wang, L., Mo, L. and Yao, Y. (2018) MicroRNA-34a suppresses breast cancer cell proliferation and invasion by targeting Notch1. Exp Ther Med, 16, 4387-4392.
23. Statello, L., Guo, C.-J., Chen, L.-L. and Huarte, M. (2021) Gene regulation by long non-coding RNAs and its biological functions. Nature Reviews Molecular Cell Biology, 22, 96-118.
24. miRagen. (2020) miragen announces internal review of preliminary topline data for the phase 2 solar clinical trial of cobomarsen in patients with cutaneous T-cell lymphoma (CTCL).
25. Anastasiadou, E., Seto, A. G., Beatty, X., Hermreck, M., Gilles, M. E., Stroopinsky, D., Pinter-Brown, L. C., Pestano, L., Marchese, C., Avigan, D. et al. (2021) Cobomarsen, an Oligonucleotide Inhibitor of miR-155, Slows DLBCL Tumor Cell Growth In vitro and In vivo. Clin Cancer Res, 27, 1139-1149.
26. Gallant-Behm, C. L., Piper, J., Dickinson, B. A., Dalby, C. M., Pestano, L. A. and Jackson, A. L. (2018) A synthetic microRNA-92a inhibitor (MRG-110) accelerates angiogenesis and wound healing in diabetic and nondiabetic wounds. Wound Repair Regen, 26, 311-323.
27. Sivori, S., Falco, M., Della Chiesa, M., Carlomagno, S., Vitale, M., Moretta, L. and Moretta, A. (2004) CpG and double-stranded RNA trigger human NK cells by Toll-like receptors: induction of cytokine release and cytotoxicity against tumors and dendritic cells. Proc Natl Acad Sci USA, 101, 10116-10121.
28. Daige, C. L., Wiggins, J. F., Priddy, L., Nelligan-Davis, T., Zhao, J. and Brown, D. (2014) Systemic delivery of a miR34a mimic as a potential therapeutic for liver cancer. Mol Cancer Ther, 13, 2352-2360.
29. Malik, S. and Bahal, R. (2019) Investigation of PLGA nanoparticles in conjunction with nuclear localization sequence for enhanced delivery of antimiR phosphorothioates in cancer cells in vitro. Journal of Nanobiotechnology, 17, 57.
30. Yi, J. and Luo, J. (2010) SIRT1 and p53, effect on cancer, senescence and beyond. Biochim Biophys Acta, 1804, 1684-1689.
31. Malik, S., Saltzman, W. M. and Bahal, R. (2021) Extracellular vesicles mediated exocytosis of antisense peptide nucleic acids. Mol Ther Nucleic Acids, 25, 302-315.
32. Chen, H., Wang, L., Zeng, X., Schwarz, H., Nanda, H. S., Peng, X. and Zhou, Y. (2021) Exosomes, a New Star for Targeted Delivery. Front Cell Dev Biol, 9, 751079.
33. Nouws, J., Wan, F., Finnemore, E., Roque, W., Kim, S. J., Bazan, I., Li, C. X., Skold, C. M., Dai, Q., Yan, X. et al. (2021) MicroRNA miR-24-3p reduces DNA damage responses, apoptosis, and susceptibility to chronic obstructive pulmonary disease. JCI Insight, 6.
34. Viteri, S. and Rosell, R. (2018) An innovative mesothelioma treatment based on miR-16 mimic loaded EGFR targeted minicells (TargomiRs). Transl Lung Cancer Res, 7, S1-S4.

1. A cationic polymeric nanoparticle comprising a cationic histidine peptide and a poly(lactic-co-glycolic acid) (PLGA) polymer, wherein the nanoparticle comprises a therapeutic agent.

2. The nanoparticle of claim 1,
(a) wherein the cationic peptide comprises a poly-L-histidine peptide;
(b) wherein the PLGA polymer and the histidine peptide are present at a ratio of about 1:1 to about 50:1;
(c) wherein the PLGA polymer and the histidine peptide are present at a ratio of about 3:2, about 4:1, about 19:1, or about 49:1; and/or
(d) wherein the PLGA polymer and the histidine peptide are present at a ratio of about 49:1.

3-5. (canceled)

6. The nanoparticle of claim 1,
(a) wherein the histidine peptide forms a cationic domain on the surface of the nanoparticle;
(b) wherein the cationic domain of the histidine peptide is about 0.5 nm to about 5 nm in diameter; and/or
(c) wherein the cationic domain of the histidine peptide is about 1.1 nm in diameter.

7. (canceled)

8. (canceled)

9. The nanoparticle of claim 1,
(a) wherein the nanoparticle is about 170 nm to about 200 nm in diameter;
(b) wherein the nanoparticle has a polydispersity index (PDI) of about 0.10 to about 0.18; and/or
(c) wherein the nanoparticle is taken up by cells via clathrin-mediated endocytosis.

10. (canceled)

11. The nanoparticle of claim 1,
(a) wherein the therapeutic agent is selected from a small molecule, a nucleic acid, a peptide nucleic acid (PNA), a mRNA, a miRNA, a siRNA, a DNA mimic, a miRNA mimic, a protein, a peptide, an antibody, a lipid, and a combinations thereof;
(b) wherein the therapeutic agent comprises a chemotherapeutic agent, a growth inhibitory agent, an anti-angiogenesis agent, an anti-neoplastic composition, or a combinations thereof;
(c) wherein the therapeutic agent is paclitaxel;
(d) wherein the therapeutic agent is a peptide nucleic acid targeting miR-155 (PNA-155); and/or
(e) wherein the therapeutic agent is a miRNA or a miRNA mimic.

12-15. (canceled)

16. The nanoparticle of claim 1, wherein the nanoparticle is prepared using an organic solvent;
(a) wherein the organic solvent comprises acetone, dichloromethane, or a combination thereof;
(b) wherein the acetone and dichloromethane are present in the organic solvent at a ratio of about 1:1 to about 50:1; and/or
(c) wherein the acetone and dichloromethane are present in the organic solvent at a ratio of about 2:1.

17-20. (canceled)

21. A pharmaceutical composition comprising the nanoparticle of claim 1 and a pharmaceutically acceptable excipient.

22. A method of preparing the cationic polymeric nanoparticle comprising a therapeutic agent of claim 1, comprising combining the cationic histidine peptide and the poly(lactic-co-glycolic acid) (PLGA) polymer in an organic solvent to form an organic phase.

23. The method of claim 22, further comprising
(a) dissolving the therapeutic agent in a first aqueous phase containing water;
(b) combining the organic phase with the first aqueous phase;
(c) subjecting the mixture of step (b) to sonication for a sufficient period of time to produce a water-in-oil emulsion;
(d) combining the water-in-oil emulsion with a second aqueous phase containing polyvinyl alcohol;
(e) subjecting the mixture of step (d) to sonication for a sufficient period of time to produce a water-in-oil-in-water emulsion;
(f) combining the water-in-oil-in-water emulsion with a third aqueous phase containing polyvinyl alcohol;
(g) allowing the organic solvent to evaporate; and/or
(h) isolating the cationic polymeric nanoparticle.

24-27. (canceled)

28. The method of claim 22,
(a) wherein the organic solvent comprises acetone, dichloromethane, or a combination thereof;
(b) wherein the acetone and dichloromethane are present in the organic solvent at a ratio of about 1:1 to about 50:1; and/or
(c) wherein the acetone and dichloromethane are present in the organic solvent at a ratio of about 2:1.

29. (canceled)

30. (canceled)

31. The method of claim 23,
(a) wherein the second aqueous phase comprises about 1% to about 20% polyvinyl alcohol;
(b) wherein the second aqueous phase comprises about 5% polyvinyl alcohol;
(c) wherein the third aqueous phase comprises about 0.1% to about 10% polyvinyl alcohol; and/or
(d) wherein the third aqueous phase comprises about 0.3% polyvinyl alcohol.

32-39. (canceled)

40. A method of treating a disease in a subject in need thereof, comprising administering to the subject a therapeutically effective amount of the nanoparticle of claim 1, thereby treating the disease in the subject in need thereof.

41. The method of claim 40, wherein the disease is cancer or autoimmune disease.

42. A method of reducing a tumor growth in a subject in need thereof, comprising administering to the subject a therapeutically effective amount of the nanoparticle of claim 1, thereby reducing the tumor growth in the subject in need thereof.

43. A method of increasing uptake of a therapeutic agent by a cell in a subject in need thereof, comprising administering to the subject a therapeutically effective amount of the nanoparticle of claim 1, thereby increasing uptake of the therapeutic agent by the cell in the subject in need thereof.

44. The method of claim 43, wherein the cell is a tumor cell.

45. The method of claim 40, wherein the nanoparticle is administered intravenously.

46. A nanoparticle formulation comprising a cationic polymeric nanoparticle and an organic solvent, wherein the cationic polymeric nanoparticle comprises a cationic histidine peptide and a poly(lactic-co-glycolic acid) (PLGA) polymer.

47. The nanoparticle formulation of claim 46,
(a) wherein the cationic peptide comprises a poly-L-histidine peptide;
(b) wherein the PLGA polymer and the histidine peptide are present at a ratio of about 1:1 to about 50:1;
(c) wherein the PLGA polymer and the histidine peptide are present at a ratio of about 3:2, about 4:1, about 19:1, or about 49:1; and/or
(d) wherein the PLGA polymer and the histidine peptide are present at a ratio of about 49:1.

48-50. (canceled)

51. The nanoparticle formulation of claim 46,
(a) wherein the organic solvent comprises acetone, dichloromethane, or a combination thereof;
(b) wherein the acetone and dichloromethane are present in the organic solvent at a ratio of about 1:1 to about 50:1; and/or
(c) wherein the acetone and dichloromethane are present in the organic solvent at a ratio of about 2:1.

52. (canceled)

53. (canceled)
